(19) United States
(12) Patent Application Publication
(10) Pub. No.: US 2010/0273660 A1
(43) Pub. Date: Oct. 28, 2010

(54) ONCOGENOMICS-BASED RNAi SCREEN AND USE THEREOF TO IDENTIFY NOVEL TUMOR SUPPRESSORS

(75) Inventors: Lars Zender, Hannover (DE); Wen Xue, Cold Spring Harbor, NY (US); Scott Powers, Greenlawn, NY (US); Scott W. Lowe, Cold Spring Harbor, NY (US)

Correspondence Address: WilmerHale/Cold Spring Harbor Laboratory, 399 Park Avenue, New York, NY, 10022, US

(73) Assignee: Cold Spring Harbor Laboratory, Cold Spring Harbor, NY (US)

(21) Appl. No.: 12/617,624

(22) Filed: Nov. 12, 2009

Related U.S. Application Data
(63) Continuation-in-part of application No. 12/072,115, filed on Feb. 21, 2008.
Application No. 12/072,115 is a continuation-in-part of application No. 11/325,218, filed on Jan. 3, 2006, now abandoned.
(60) Provisional application No. 60/641,043, filed on Jan. 3, 2005.
(60) Provisional application No. 60/686,609, filed on Jun. 1, 2005.
(60) Provisional application No. 61/113,866, filed on Nov. 12, 2008.

(51) Int. Cl. C40B 20/06; C07K 16/00; C40B 50/04; C40B 40/06; C12N 5/09; C12Q 1/68; G01N 33/574

(52) U.S. Cl. 506/5; 530/389.7; 506/25; 506/16; 435/375; 435/6; 436/64

(57) ABSTRACT

In some aspects, the invention provides a genetically tractable in situ non-human animal model for hepatocellular carcinoma. The model is useful, inter alia, in understanding the molecular mechanisms of liver cancer, in understanding the genetic alterations that lead to chemoresistance or poor prognosis, and in identifying and evaluating new therapies against hepatocellular carcinomas. The liver cancer model of this invention is made by altering hepatocytes to increase oncogene expression, to reduce tumor suppressor gene expression or both and by transplanting the resulting hepatocytes into a recipient non-human animal.

The present invention also provides methods for identifying and validating tumor suppressor genes by screening pools of shRNAs that target genomic regions deleted in human cancers, such as human hepatocellular carcinomas. The present invention also provides validated tumor suppressor genes, and methods of inhibiting cell proliferation and/or tumor growth, for example by expression of such tumor suppressor genes.

### REFERENCE TO RELATED APPLICATIONS

This application is a continuation-in-part of U.S. application Ser. No. 12/072,115, filed on Feb. 21, 2008, which is a continuation-in-part of U.S. application Ser. No. 11/325,218, filed on Jan. 3, 2006, which claims priority to U.S. Provisional Application No. 60/641,043, filed on Jan. 3, 2005, and U.S. Provisional Application No. 60/686,609, filed on Jun. 1, 2005, and this application claims the benefit of the filing date of U.S. Provisional Patent Application No. 61/113,866, filed Nov. 12, 2008. Each of the foregoing applications is hereby incorporated by reference in its entirety.

### GOVERNMENT INTERESTS

This invention was made in part with government support under Grant Nos. CA13106, CA87497, and CA105388, awarded by the National Institutes of Health. Accordingly, the United States Government has certain rights to the invention.

All patents, patent applications and publications cited herein are hereby incorporated by reference in their entirety.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD OF THE INVENTION

This invention provides a genetically tractable in situ non-human animal model for liver cancer and specifically hepatocellular carcinoma. The model is useful, inter alia, in understanding the molecular mechanisms of liver cancer, in understanding the genetic alterations that lead to chemoresistance or poor prognosis, and in identifying and evaluating new therapies against hepatocellular carcinomas.

This invention also provides, inter alia, methods and libraries useful for efficiently identifying tumor suppressor genes, and tumor suppressor genes identified using such methods and libraries.

### BACKGROUND INFORMATION

Cancer is the second leading cause of death in industrial countries. More than 70% of all cancer deaths are due to carcinomas, i.e., cancers of epithelial organs. Most carcinoma tumors show initial or compulsory chemoresistance. This property makes it very difficult to cure these tumors when they are detected in progressed stages. Primary forms of liver cancers include hepatocellular carcinoma, biliary tract cancer and hepatoblastoma. Hepatocellular carcinoma is the fifth most common cancer worldwide but, owing to the lack of effective treatment options, constitutes the leading cause of cancer deaths in Asia and Africa and the third leading cause of cancer death worldwide. Parkin et al. “Estimating the world cancer burden: Globocan 2000.” Int. J. Cancer 94, 153-156 (2001).

The risk factors for liver cancer include excessive alcohol intake or other toxins, such as iron, aflatoxin B1 and also the presence of other infections such as hepatitis B and C. Alison & Lovell. “Liver cancer: the role of stem cells.” Cell Prolif. 38, 407-421 (2005). The only curative treatments for hepatocellular carcinoma are surgical resection or liver transplantation, but most patients present with advanced disease and are not candidates for surgery. To date, systemic chemotherapeutic treatment is ineffective against hepatocellular carcinoma, and no single drug or drug combination prolongs survival. Llovet et al. “Hepatocellular carcinoma.” Lancet 362, 1907-1917. (2003). However, despite its clinical significance, liver cancer is understudied relative to other major cancers.

One of the difficulties in identifying appropriate therapeutics for tumor cells in vivo is the limited availability of appropriate test material. Human tumor lines grown as xenographs are unphysiological, and the wide variation between human individuals, not to mention treatment protocols, makes clinical studies difficult. Consequently, oncologists are often forced to perform correlative studies with a limited number of highly dissimilar samples, which can lead to confusing and unhelpful results.

Non-human animal models provide a useful alternative to studies in humans and to human tumor cell lines grown as xenographs, as large numbers of genetically-identical individuals can be treated with identical regimens. Moreover, the ability to introduce germline mutations that affect oncogenesis into these animals increases the power of the models.

To investigate the basic mechanisms of carcinogenesis and to test new potential cancer agents and therapies, however, realistic carcinoma-non-human animal models are urgently needed. So far there have been two major ways to create carcinoma non-human animal models: (i) the generation of transgenic or chimeric non-human animals that express oncogenes under the control of a tissue specific promoter and (ii) carcinomas that were induced by chemical carcinogens. Both approaches have several disadvantages.

Current animal models for cancer are based largely on classical transgenic approaches that direct expression of a particular oncogene to an organ of choice using a tissue specific promoter. See, e.g., Wang et al. “Activation of the Met receptor by cell attachment induces and sustains hepatocellular carcinomas in transgenic mice.” J. Cell Biol. 153, 1023-1034 (2001). Although such models have provided important insights into the pathogenesis of cancer, they express the active oncogene throughout the entire organ, a situation that does not mimic spontaneous tumorigenesis. Moreover, incorporation of additional lesions, such as a second oncogene or loss of a tumor suppressor, requires genetic crosses that are time consuming and expensive, and again produce whole tissues that are genetically altered. Finally, traditional transgenic and knockout strategies do not specifically target liver progenitor cells, which may be the relevant initiators of the disease.

Cancer therapies that directly target oncogenes are based on the premise that cancer cells require continuous oncogenic signaling for survival and proliferation. Non-human animal models expressing oncogenes in genetic backgrounds that lack, or have down-regulated, tumor suppressor genes can thus serve as valuable tools to study tumor initiation, maintenance, progression, treatment and regression. However, responses to the targeting drugs are often heterogeneous, and chemoresistance and other resistance is a problem. Because most anticancer agents were discovered through empirical screens, efforts to overcome resistance are hindered by a limited understanding of why these agents are effective and when and how they become less or non-effective.

Furthermore, although cancer usually arises from a combination of mutations in oncogenes and tumor suppressor genes, the extent to which tumor suppressor gene loss is required for the maintenance of established tumors is poorly understood.

Variations in both non-human animal strains and promoters used to drive expression of oncogenes complicate the interpretation of cancer mechanistics and treatment analyses. Firstly, intercrossing strategies to obtain non-human animals of the desired genetic constellation are extremely time consuming and costly. Secondly, the use of certain cell-selective promoters can result in a cell-bias for tumor initiation. For example, the mouse mammary tumor virus (MMTV) promoter and the Whey Acidic Protein (WAP) promoter are commonly used to model breast cancer development in mice, and yet may not target all subtypes of mammary epithelia, i.e., stem cell and non-stem cells. Finally, a homogenous expression of the respective oncogene in all epithelial cells of an organ creates an unphysiological condition, as tumors are known to originate within genetic-mosaics.

An additional difficulty in identifying and evaluating the efficacy of cancer agents on tumor cells and understanding the molecular mechanisms of the cancers and their treatment in the current non-human animal models in vivo is the limited availability of appropriate material.

It is therefore important to use a valid model to identify new therapeutics for the treatment of liver cancer.

Investigation of the role of oncogenes or tumor suppressor genes in tumorigenesis can be facilitated by specifically silencing, or preventing from exerting its presence, the particular gene of interest. One such silencing means is through “RNA interference” or “RNAi.” RNAi stems from a phenomenon observed in plants and worms whereby double-stranded RNA (dsRNA) blocks gene expression in a specific and post-transcriptional manner. The dsRNA is cleaved by an RNAse III enzyme “DICER” into a 21-23 nucleotide small interfering RNA (siRNA). These siRNAs are incorporated into a RNA-induced silencing complex (RISC) that identifies and silences RNA complimentary to the siRNA. Without being bound by theory, RNAi appears to involve silencing of cytoplasmic mRNA by triggering an endonuclease cleavage, promoting translation repression, or possibly accelerating mRNA decapping (Valencia-Sanchez et al., 2006, Genes & Development 20: 515-524). Biochemical mechanisms of RNAi are currently an active area of research.

Three mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC complex to silence target mRNA expression.

The second mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). The endogenous RNAi pathway is comprised of three RNA intermediates: a a long, largely single-stranded primary miRNA transcript (pri-mRNA), a precursor miRNA transcript having a stem-and-loop structure and derived from the pri-mRNA (pre-miRNA), and a mature miRNA. The miRNA gene is transcribed by an RNA polymerase II promoter into the pri-mRNA transcript, which is then cleaved to form the pre-miRNA transcript (Fewell et al., supra). The pre-miRNA is transported to the cytoplasm and is cleaved by DICER to form mature miRNA. miRNA then interacts with the RISC in the same manner as siRNA. shRNAs, which mimic pre-miRNA, are transcribed by RNA Polymerase II or III as single-stranded molecules that form stem-loop structures. Once produced, they exit the nucleus, are cleaved by DICER, and enter the RISC complex as siRNAs.

The third mechanism is identical to the second mechanism, except that the shRNA is modeled on pri-miRNA (shRNAmir), rather than pre-miRNA transcripts (Fewell et al., supra). An example is the miR-30 miRNA construct. The use of this transcript produces a more “physiological” shRNA that reduces toxic effects. The shRNAmir is first cleaved to produce shRNA, and then cleaved again by DICER to produce siRNA. The siRNA is then incorporated into the RISC for target mRNA degradation.

RNAi has been used to successfully identify and suppress target genes associated with tumorigenesis. For example, expression of microRNA-based shRNA specific to Trp53 produces “potent, stable, and regulatable gene knock-down in cultured cells . . . even when present at a single copy in the genome” (Dickins et al., 2005, Nature Genetics 37: 1289-1295). The tumors induced by the p53 knockdown regress upon re-expression of Trp53. Id. The suppression of the Trp53 gene expression by shRNA is also possible in stem cells and reconstituted organs derived from those cells (Hemann et al., 2003, Nature Genetics 33: 396-400). Moreover, the extent of inhibition of p53 function by the shRNA correlates with the type and severity of subsequent lymphomagenesis. Id.

However, there are conflicting views on which method of introducing and using RNAi mechanism is most effective. Some studies emphasize siRNA's several drawbacks, including transient effects, difficulty in delivery to nondividing primary cells, and concentration-dependent off-target effects. shRNAs expressed from viral vectors “are more versatile, allowing . . . stable integration, germline transmission, and the creation of in vivo animal models (Fewell et al., supra). shRNA is also more suitable for hard-to-transfect cells, due to its infection-based delivery, and has decreased concentration-dependent off-target effects. Id. In comparison with shRNA, shRNAmir is more efficiently processed into siRNA and produces a more consistent silencing of mRNA than shRNA. Id.

Despite the advantages of shRNA, other studies maintain that use of siRNA for RNAi purposes is emerging more rapidly than the use of shRNA (Lu et al., supra), partly because of the “increased effort required to construct shRNA expression systems before selection of active sequences and verification of biological activity are obtained.” Id. It is often time consuming and expensive to both construct shRNA expression cassettes and incorporate them into viral delivery systems. Id. On the contrary, use of synthetic oligonucleotides allows for rapid screening and studying of siRNA sequences and matching genes. Id. Moreover, recent studies investigating in vivo applications of RNAi focus on non-viral delivery of siRNA constructs as opposed to viral delivery of shRNA constructs, as viral vectors often raise concerns about safety and immunogenecity (Lu et al., supra; Vohies et al., 2007, Expert Rev. Anticancer Ther. 7: 373-382). In sum, there is no established method of RNAi that consistently produces the most effective RNA silencing.

Studies also vary in their use of genome-wide collections of pooled shRNA vectors versus small sets of shRNA vectors that target a specific gene family. The use of large shRNA libraries may lead to difficulties in measuring the relative abundance of each individual shRNA vector in a complex population of cells infected with thousands of vectors. In addition, the smaller scaled experiments allow “screening for relatively labor-intensive phenotypes.” Id. Pooled screens also pose several technological hurdles, such as obtaining uniform pools of viruses, creating robust design algorithms that enable gene knockdown at a single-copy level, and preventing large numbers of false positives (Fewell et al., supra). On the other hand, the use of barcodes, or unique sequence of nucleotides incorporated into each shRNA vector, allows for more accurate quantification of specific shRNAs in pooled analyses (Bernards et al., 2006, Nature Methods 3: 701-706). Moreover, larger shRNA library screens can be used to select for long-term phenotypes while smaller shRNA screens are mainly limited to “short-term” readouts. Id. Given the various benefits and drawbacks of both large and small scale screens, there is no suggestion that use of one method or the other is the most effective strategy for successful RNAi.

Finally, although certain tumor suppressors such as p53 are well-studied, the importance of other individual tumor suppressors is still unknown. As such, the extent of overall tumor suppressor gene loss required for maintaining tumors is poorly understood. Moreover, although there is potential to utilize Myc overexpression to investigate novel tumor suppressor genes, few scientists have so far been able to do so.

Established approaches for the investigation of novel oncogenes and tumor suppressor genes using RNAi are thus unavailable.

### SUMMARY OF THE INVENTION

In some aspects, the present invention provides in vivo and in vitro systems and methods for the study of the effects of tumorigenesis, tumor maintenance, tumor regression and altered expression of a gene activity, on the descendants of embryonic liver progenitor cells, or primary hepatocytes, that have been engineered to produce hepatocellular carcinomas.

In some aspects of the invention, liver cancer models are made by altering hepatocytes to increase oncogene expression, to reduce tumor suppressor gene expression or both and by transplanting the resulting hepatocytes into a recipient non-human animal. The spontaneous mutations arising in tumors initiated by different oncogenic lesions are compared to alterations observed in human cancers. Preferably, the transplanting is carried out so that the hepatocytes engraft the liver of the animal and a liver cancer tumor develops there from at least one of the altered hepatocytes. Less preferably, the altered hepatocytes are transplanted subcutaneously into a non-human animal so as to develop a tumor.

In some embodiments, the non-human animal model of hepatocellular carcinoma embodied herein is useful for identifying molecular targets for drug screening, for identifying interacting gene activities, for identifying therapeutic treatments and for identifying candidates for new therapeutic treatments. The invention also provides methods and non-human animals produced by the methods that are useful for understanding liver cancer and its treatments, and in particular, for identifying and studying inhibitors and activators associated with liver tumor cell growth and growth inhibition, cell death through apoptotic pathways, and changes in apoptotic pathway components that affect drug sensitivity and resistance in tumorigenic cells.

The genetically tractable, transplantable in situ liver cancer models described herein are characterized by genetically defined hepatocellular carcinomas that are preferably traceable by external green fluorescent protein (GFP) imaging. To further characterize the genetic defects in these tumors, gene expression profiling, e.g., representational oligonucleotide microarray analysis (ROMA), can be used to scan the carcinomas for spontaneous gains and losses in gene copy number. Detecting genomic copy number changes through such high resolution techniques can be useful to identify oncogenes (amplifications or gains) or tumor suppressor genes (deletions or losses). Identification of overlapping genomic regions altered in both human and mouse gene array datasets may further aid in pinpointing of regions of interest that can be further characterized for alterations in RNA and protein expression to identify candidates are most likely to contribute to the disease phenotype and to be the “driver gene” for amplification.

Using “forward genetics” in combination with gene expression profiling (e.g., ROMA) and the non-human animal models of this invention, important insights into the molecular mechanisms of hepatocarcinogenesis, growth, maintenance, regression and remission can be obtained. The models of the invention can directly evaluate the potency of various oncogenes in producing anti-apoptotic phenotypes, and various tumor suppressor genes in producing apoptotic phenotypes. Candidate oncogenes or tumor suppressors can be rapidly validated in the mouse model of the invention by overexpression, or by using stable RNAi technology, respectively. The invention is also useful in analyzing and evaluating genetic constellations that confer chemoresistance or poor prognosis. Furthermore, the invention is useful for identifying and evaluating new therapies for the treatment of carcinomas.

In one aspect the present provides methods for identifying genes that are associated with tumorigenesis, such as a tumor suppressor gene, or oncogenes. Such methods are based on generating and/or using focused nucleic acid libraries (such as cDNA libraries, RNAi libraries, and the like), that containg sequences that are contained within or targted to genomic regions that are altered in cancer cells. The present invention thus uses oncogenomic approaches to allow focused screening for cancer associated genes. For example, the present invention provides libaries of cDNAs that are encoded by genes amplified in cancer cells. Such libaries are useful for identifying oncogenes. The present invention also provides libaries of RNAi molecules, such as shRNAs, that target genes or genomic regions deleted in cancer cells, or that target genes that are methylated in cancer cells. Such libaries are useful for identifying tumor suppressor genes. The present invention provides, inter alia, such focused libaries, methods of identifying tumor suppressor genes and oncogenes using such focused libaries, and oncogenes and tumor suppressor genes identified using such focused libraries. The libraries and methods can be used to identify oncogenes and tumor suppressor genes in any type of cancer cell. In some embodiments, such as those illustrated in the Examples, the libraries and methods can be used to identify oncogenes and tumor suppressor genes in liver cancer cells and breast cancer cells.

In one aspect, the method comprises (1) transfecting a plurality of hepatocytes with a library of RNAi molecules, wherein each of the RNAi molecule inhibits expression of a target gene, (2) transplanting the transfected cells into a recipient non-human animal, and (3) identifying the RNAi molecule that give rise to liver cancer in the recipient animal. The RNAi molecule may be identified, for example, by isolating the genomic DNA from the cancer cells, amplifying the transfected RNAi molecule by PCR, and sequencing the amplified DNA. In certain embodiments, the RNAi library comprises RNAi molecules that inhibit the expression of genes known to be up-regulated or down-regulated in human cancers, such as liver cancer. In certain embodiments, the RNAi library comprises RNAi molecules that inhibit the expression of genes that are deleted in a cancer genome. In particular, the RNAi library may comprise RNAi molecules that inhibit the expression of genes that are located within a recurrent genomic deletion found in two or more cancer genomes.

In another aspect, the present invention provides a method for generating a library of RNAi molecules, such as an shRNA libray, that is useful for identifying tumor suppressor genes, the method comprising: identifying one or more genes or genomic deletions, such as focal genomic deletions, in a population of cancer cells; cloning one or more RNAi molecules, such as shRNAs molecules, that target sequences located within one or more of the genomic deletions into an expression vector; and pooling the expression vectors to generate a library of RNAi molecules, such as an shRNA library, that is useful for identifying tumor suppressor genes.

In another aspect, the present invention provides a method for generating a library of RNAi molecules, such as an shRNA libray, that is useful for identifying tumor suppressor genes, the method comprising: identifying one or genes or genomic regions that are methylated in a population of cancer cells; cloning one or more RNAi molecules, such as shRNAs molecules, that target sequences located within one or more of the genes or genomic regions that are methylated into an expression vector; and pooling the expression vectors to generate a library of RNAi molecules, such as an shRNA library, that is useful for identifying tumor suppressor genes.

In another aspect, the present invention provides a method for generating a library of cDNA molecules that is useful for identifying oncogenes, the method comprising: identifying one or genes or genomic regions that are amplified in a population of cancer cells; cloning one or more cDNA molecules, that target sequences located within one or more of the genomic regions that are amplified into an expression vector; and pooling the expression vectors to generate a library of cDNA molecules that is useful for identifying oncogenes.

The population of cancer cells can be any population of cancer cells. In some embodiments the population of cancer cells is a population of liver cancer cells, such as mouse or human hepatocellular carcinoma cells. In some embodiments, the population of cancer cells is a population of breast cancer cells, such as mouse or human breast cancer cells.

Genomic deletions or amplifications can be identified using methods known in the art, including but not limited to, representational oligonucleotide microarray analysis (“ROMA”). Methylated regions of genomic DNA can be identified using methods known in the art, including, but not limited to global and gene-specific methylation analyses methods such as methods that involve assessing the level of methyl cytosines (such as chromatographic methods), methods that use methylation sensitive restriction enzymes to digest DNA followed by Southern detection or PCR amplification, bisulfite reaction based methods, methylation specific PCR (MSP), bisulfite genomic sequencing PCR, Restriction Landmark Genomic Scanning for Methylation (RLGS-M), methods for detecting CpG islands such as CpG island microarray based methods, chromatin immuno precipitation—based methods such as DNA Methylation ChIP (meDIP), and the like.

In some embodiments, the genomic deletions or amplifications or methylated genomic regions are identified from genome database information or from other sources that describe genomic deletions and amplfications or methylated regions that associated with one or more cancers, such as mouse or human cancers.

In some embodiments, the expression vector is a viral vector and the method of transforming the mouse embryonic hepatocytes comprises infection. In other embodiments the expression vector may be a non-viral expression vector and the methods of transforming the mouse embryonic hepatocytes comprises transfection.

In some embodiments, the expression vector further comprises a reporter, such as green fluorescent protein (GFP).

In some embodiments, the library, such as the cDNA or RNAi library, comprises up to about 100 different sequences. In some embodiments, the library, such as the cDNA or RNAi library, comprises up to about 90 different sequences. In some embodiments, the library, such as the cDNA or RNAi library, comprises up to about 80 different sequences. In some embodiments, the library, such as the cDNA or RNAi library, comprises up to about 70 different sequences. In some embodiments, the library, such as the cDNA or RNAi library, comprises up to about 60 different sequences. In some embodiments, the library, such as the cDNA or RNAi library, comprises up to about 55 different sequences. In some embodiments, the library, such as the cDNA or RNAi library, comprises up to about 50 different sequences. In some embodiments, the library, such as the cDNA or RNAi library, comprises up to about 45 different sequences. In some embodiments, the library, such as the cDNA or RNAi library, comprises up to about 40 different sequences. In some embodiments, the library, such as the cDNA or RNAi library, comprises up to about 35 different sequences. In some embodiments, the library, such as the cDNA or RNAi library, comprises up to about 30 different sequences. In some embodiments, the library, such as the cDNA or RNAi library, comprises about 48 different sequences.

In some aspects, the present invention provides libraries of cDNA or RNAi molecules, such as shRNA libraries, generated using the methods described above.

For example, in one aspect, the present invention provides an shRNA library that comprises, or consists essentially of, or consists of shRNA molecules that target mRNAs transcribed from one or more genes located within one or more cancer-associated genomic deletions, such as genomic deletions associated with liver cancer or breast cancer, or located within one or more genomic regions that are methylated in cancer cells, such as liver cancer or breast cancer cells.

In one aspect, the shRNA molecules in the library target mRNAs transcribed from one or more genes selected from the 362 genes listed in Table 3. In another aspect, the the shRNA molecules in the library target mRNAs transcribed from one or more genes located within one or more of the 58 cancer-associated focal genomic deletions listed in Table 3.

In other aspects, the present invention provides methods for identifying tumor suppressor genes or oncogens using the libraries described herein.

For example, the present invention provides a method of identifying a tumor suppressor gene, comprising: identifying one or more genomic deletions, such as focal genomic deletions, in a population of cancer cells; cloning one or more RNAi molecules, such as shRNAs, that target sequences located within the one or more genomic deletions into an expression vector; pooling the expression vectors to generate an RNAi library; transforming the RNAi library into mouse embryonic hepatocytes; introducing transformed mouse embryonic hepatocytes into mice; amplifying one or more RNAi molecules from a tumor generated in a transformed mouse; and identifying a gene sequence to which the shRNA is complementary; whereby the gene identified can be a tumor suppressor gene. In some embodiments, the RNAi molecules are shRNA molecules and the RNAi library is an shRNA library.

In another embodiment, the present invention provides a method of identifying a tumor suppressor gene, comprising: identifying one or more genomic regions or genes that are methylated in a population of cancer cells; cloning one or more RNAi molecules, such as shRNAs, that target sequences located within the one or more methylated genomic regions or genes into an expression vector; pooling the expression vectors to generate an RNAi library; transforming the RNAi library into mouse embryonic hepatocytes; introducing transformed mouse embryonic hepatocytes into mice; amplifying one or more RNAi molecules from a tumor generated in a transformed mouse; and identifying a gene sequence to which the shRNA is complementary; whereby the gene identified can be a tumor suppressor gene. In some embodiments, the RNAi molecules are shRNA molecules and the RNAi library is an shRNA library.

In another embodiment, the present invention provides a method of identifying an oncogene, the method comprising:identifying a genomic amplification in a population of cancer cells; cloning one or more cDNAs encoded by genes located within the genomic amplification into an expression vector; pooling the expression vectors generated in step (b) to generate a library of cDNAs; transforming the library of cDNAs into mouse embryonic hepatocytes; introducing the transformed mouse embryonic hepatocytes into mice; amplifying a cDNA present in the library of cDNAs from a tumor cell formed in the mice; and determining the sequence of the cDNA; thereby identifying an oncogene.

It should be noted that in all of the above embodiments, the library of nucleic acid molecules, such as cDNA or shRNA molecules can alternatively be transformed into any cell type that be introduced into mice and which can form a tumor. In preferred embodiments, the the library of nucleic molecules is transformed into mouse embryonic hepatocytes, which can be introduced into mice and which can form tumors in mice. Such a mouse cancer model is described herein and also in Zender et al. 2006 (Cell. 125; p 1253-1267), the contents of which are hereby incorporated by reference.

In some embodiments, the mouse embryonic hepatocytes into which the cDNA or RNAi library is transformed are p53-negative and/or Myc positive.

cDNAs or shRNAs that are identified in the above methods can be validated by introduction into cells and assessed (e.g. for gene knockdown or for over-expression), for example by immunoblotting or RT-Q-PCR. If positive, the individual cDNAs or shRNAs can be further evaluated for their activities in mice, for example by assessing their effects on tumor formation. To confirm the involvement of the target gene of an shRNA, new hairpins can be created against the same gene and put back into mice to rule out off-target effects. These newly created hairpins can be evaluated through knockdown as well.

Candidate tumor suppressors can be further assessed by in vitro validation processes to ascertain the mechanism by which knockdown of the putative tumor suppressors is tumorigenic. Such processes can elucidate whether the tumorigenesis is due to apoptotic defects or proliferation advantage. For example, response to growth factor withdrawal, DNA damage response to cytotoxic drugs, and/or activity of downstream targets can be examined. Similarly, candidate oncogenes can be further assessed by in vitro validation processes to ascertain the mechanism by which their expression or over-expression is tumorigenic.

In another aspect, the invention discloses tumor suppressor genes whose down-regulation is associated with liver cancer, for example as described in Example 2.

In another aspect, the invention discloses tumor suppressor genes whose down-regulation is associated with breast cancer.

For example, in one embodiment, the present invention provides a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9. Such tumor suppressor genes can be associated with liver cancer and/or breast cancer, as described in Example 2 and Example 3.

In another aspect, the invention discloses an oncogene whose over-expression and/or amplification is associated with liver cancer and breast cancer, wherein the oncogene is EIF5A2.

In other aspects, the present invention provides methods for inhibiting cell proliferation. For example, the present invention provides methods for inhibiting cell proliferation comprising upregulating expression of a gene selected from the group consisting of XPO4, FGF6, WDR49, ARMCX2, FSTL5, NRSN2, WDR37, ARMCX1, GJD4, ZBBX, GLO1, DDX20, SET, and BTBD9 in a cell. In some embodiments, the gene is XPO4. In some embodiments, the step of upregulating XPO4 expression comprises introducing into the cell a nucleic acid molecule encoding a physiologically active XPO4 polypeptide. In some embodiments, the cell is a cancer cell, such as a liver cancer cell or a breast cancer cell. The cancer cell my be in vitro or may in a subject, such as a mouse or human subject.

In some embodiments, the present invention provides a method of inhibiting cell proliferation comprising administering to a cell a a physiologically active polypeptide encoded by a gene selected from the group consisting of XPO4, FGF6, WDR49, ARMCX2, FSTL5, NRSN2, WDR37, ARMCX1, GJD4, ZBBX, GLO1, DDX20, SET, and BTBD9, or an analog thereof, thereby inhibiting cell proliferation. In some embodiments, the method comprises administering a physiologically active XPO4 polypeptide, or an analog thereof, to the cell. In some embodiments, the cell is a cancer cell, such as a liver cancer cell or a breast cancer cell. The cancer cell my be in vitro or may in a subject, such as a mouse or human subject.

Another aspect of the invention is a method of treating cancer comprising identifying decreased expression of at least one tumor suppressor gene in a cancer cell as compared with that in a normal cell and upregulating the tumor suppressor. Upregulation can be effected by introducing the tumor suppressor into the cancer cell. In a particular embodiment, the tumor suppressor protein or a physiologically active fragment, analog, or variant thereof is administered to the cell. In another particular embodiment, a nucleotide sequence encoding the tumor suppressor or a fragment or variant thereof encoding a physiologically active polypeptide is introduced into the cancer cell and expressed. In yet another embodiment, known upstream factors of an identified tumor suppressor is modulated to increase the tumor suppressor expression.

More specifically, an embodiment of the invention is a method for treating cancer comprising identifying in a cancer cell decreased expression of at least one tumor suppressor gene identified herein as compared with that in a normal cell and upregulating the tumor suppressor. More particularly, said tumor suppressor gene for which the status is determined is selected from XPO4, FGF6, WDR49, ARMCX2, FSTL5, NRSN2, WDR37, ARMCX1, GJD4, ZBBX, GLO1, DDX20, SET, and BTBD9. Known substrates for XPO4, for example, are SMAD3 and EIF5A1. Thus, one embodiment of the invention is modulating downstream factors to counteract tumor suppressor inactivation.

Another embodiment of the invention is reducing or inhibiting cell proliferation by upregulating a tumor suppressor. In a preferred embodiment, the cell is a cancer cell. In another preferred embodiment, the tumor suppressor is selected from XPO4, FGF6, WDR49, ARMCX2, FSTL5, NRSN2, WDR37, ARMCX1, GJD4, ZBBX, GLO1, DDX20, SET, and BTBD9.

In one embodiment, cell proliferation is reduced or inhibited by upregulating or downregulating downstream factors, such as tumor suppressor substrates and other components in the tumor suppressor's signaling pathway. For example, in cells having reduced XPO4 expression or loss of XPO4 function, downregulation or inhibition of the XPO4 substrates, EIF5A and/or SMAD3, can reduce or inhibit cell proliferation. Therefore in one embodiment of the invention, EIF5A and/or SMAD3 are downregulated or inhibited. The same approach can be applied to any of the tumor suppressor targets identified by the present invention. In a preferred embodiment, inhibition of tumor suppressor substrates or other downstream factors is achieved by RNAi. In other embodiments, upregulation of the downstream factors can reduce or inhibit cell proliferation.

Another aspect of the invention is a method of treating cancer comprising the steps of determining the status in cancerous tissue of one or more of the tumor suppressor genes described herein or identified by a screening method described herein, and if any of the tumor suppressors shows a decreased expression or activity in cancerous tissue in comparison to a control (such as a normal tissue), increasing the expression of said tumor suppressor(s).

In one embodiment, the expression or activity of a tumor suppressor is increased by introducing the tumor suppressor into the cancerous tissue. In a particular embodiment, the tumor suppressor protein or a physiologically active fragment, analog, or mutant thereof is administered. In another particular embodiment, the tumor suppressor gene or a fragment or mutant thereof that encodes a physiologically active polypeptide is introduced into the cancer tissue and expressed. In yet another embodiment, known upstream factors of an identified tumor suppressor is modulated to increase the tumor suppressor expression.

Another aspect of the invention is a method of treating cancer comprising determining in cancerous tissue the expression of one or more tumor suppressor genes described herein or identified by the screening method described herein, the expression of which gene or genes are increased or decreased in comparison to a control (e.g., a normal tissue), and administering a therapeutic agent that is known to be effective in treating such cancers that are associated with the increased or decreased expression of such gene or genes. Alternatively, an aspect of the invention is a method of treating cancer comprising the steps of determining in cancerous tissue the expression of one or more tumor suppressor genes described herein or identified by the screening method described herein, the expression of which gene or genes are decreased in comparison to a control (e.g., a normal tissue), and administering a therapeutic agent that is known not to antagonize the gene or genes identified herein.

In other embodiments, the present invention provides methods for inhibiting cell proliferation by inhibiting the expression and/or activity of the EIF5A2 gene or the EIF5A2 gene product in the cell. In some embodiments, the method comprises introducing into the cell an interfering RNA molecule that targets EIF5A2, such as an shRNA or an siRNA molecule. In some embodiments, the cell is a cancer cell, such as a liver cancer cell or a breast cancer cell. The cancer cell my be in vitro or may in a subject, such as a mouse or human subject.

In another aspect, the invention discloses a method for identifying a subject who is suffering from or susceptible to cancer, comprising: determining the expression level of one or more identified tumor suppressor genes. A decrease in the expression level of the tumor suppressor gene, as compared to a control, is indicative of a subject suffering from or susceptible to liver cancer.

In one embodiment, the method comprises obtaining a tissue sample from the subject, determining the expression of one or more tumor suppressors selected from the group consisting of XPO4, FGF6, WDR49, ARMCX2, FSTL5, NRSN2, WDR37, ARMCX1, GJD4, ZBBX, GLO1, DDX20, SET, and BTBD9 in the tissue sample, and comparing said activity to that in normal tissue, wherein the subject is diagnosed with cancer if the activity of any one of tumor suppressor is substantially decreased or is not detectable in the tissue sample.

In another embodiment, the method comprises determining the expression of one or more tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9 in the tissue sample and comparing said expression to that in normal tissue, wherein the subject is diagnosed with cancer if said expression is substantially decreased or no expression is detected in the tissue sample.

In one embodiment, the present invention provides a method for determining whether a subject is suffering from, or at risk of developing, liver or breast cancer, the method comprising obtaining genomic DNA from the subject and determining whether the genomic DNA contains a deletion of one or more of genes selected from the group consisting of XPO4, FGF6, WDR49, ARMCX2, FSTL5, NRSN2, WDR37, ARMCX1, GJD4, ZBBX, GLO1, DDX20, SET, and BTBD9, whereby a deletion of one or more of the genes indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

In other embodiments, the present invention provides a method for determining whether a subject is suffering from, or at risk of developing, liver or breast cancer, the method comprising obtaining a sample comprising mRNA from the subject, and measuring the level of an mRNA selected from the group consisting of XPO4, FGF6, WDR49, ARMCX2, FSTL5, NRSN2, WDR37, ARMCX1, GJD4, ZBBX, GLO1, DDX20, SET, and BTBD9 mRNAs in the sample, whereby a level of expression of one or more of the mRNAs that is lower than the level of expression of the mRNA in a control subject, indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

In other embodiments, the present invention provides a method for determining whether a subject is suffering from, or at risk of developing, liver or breast cancer, the method comprising obtaining a sample from the subject and measuring the level of a protein selected from the group consisting of the proteins encoded by the XPO4, FGF6, WDR49, ARMCX2, FSTL5, NRSN2, WDR37, ARMCX1, GJD4, ZBBX, GLO1, DDX20, SET, and BTBD9 genes in the sample, whereby a level of the protein that is lower than the level of the same protein in a control subject, indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

The sample used in the above methods can be e.g., a biopsy specimen such as samples from needle biopsy, a sample of bodily fluid, e.g., serum, plasma, urine, or ejaculate. Normal tissue used as negative control can be tissue from any individual not diagnosed with cancer and of the same species as the subject. Such subject does not show any symptoms or known biological marker for the cancer being tested for. Preferably, the quantitative measurement from the tissue sample is compared to the values obtained from more than one normal tissue.

The tumor suppressor proteins described herein can be detected using monoclonal antibodies, for example commercially available antibodies or antibodies prepared using methods known in the art. The tumor suppressor genes and/or mRNAs described herein can be detected using various methods available in the art, including but not limited to, quantitative PCR methods.

Another aspect of the present invention is a kit useful for the above methods e, e.g., for use in detecting a decrease in expression of or lack of a tumor suppressor gene identified herein. In one embodiment, the present invention provides a kit, e.g., a compartmentalized carrier including a first container containing a pair of primers for amplification of a tumor suppressor, a second container containing a pair of primers for amplification of a region in a reference gene, and a third container containing a first and second oligonucleotide probe specific for the amplification of the biomarker and the region of the reference gene, respectively. A reference gene may be any gene that is consistently expressed in any tissue regardless of whether the tissue is cancerous.

Yet another aspect of the invention is a pharmaceutical composition comprising a therapeutic agent for the treatment of cancer, which composition has specific utility to treat such cancer that has certain status regarding one or more tumor suppressors identified using the method described herein.

One embodiment of the invention is a pharmaceutical composition for the treatment of cancer in which the expression or activity of said tumor suppressor is less than a control (e.g., in normal tissue), comprising a tumor suppressor protein or a physiologically active fragment, analog, or mutant thereof. Another particular embodiment is a pharmaceutical composition for the treatment of cancer in which the expression or activity of a tumor suppressor is less than a control (e.g., in normal tissue), comprising a tumor suppressor gene or a fragment or mutant thereof that encodes a physiologically active polypeptide is introduced into the cancer tissue and expressed. In yet another embodiment, a pharmaceutical composition comprises one or more therapeutic agents that modulate known upstream factors of an identified tumor suppressor to increase the tumor suppressor expression.

In another aspect, the invention provides compositions, such as pharmaceutical compositions, that can be used to inhibit proliferation of a cell, such a cancer cell, for example a liver cancer cell or a breast cancer cell. Such compositions can comprise an agent that upregulates expression of a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9, or can comprise a nucleic acid sequence that encodes a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9 or an analog thereof, or can comprise a polypeptide encoded by a tumor suppressor gene selected from the group consisting of XPO4, FGF6, WDR49, ARMCX2, FSTL5, N. RSN2, WDR37, ARMCX1, GJD4, ZBBX, GLO1, DDX20, SET, and BTBD9, or an analog thereof. In one embodiment, the present invention provides a composition comprising a protein encoded by gene selected from the group consisting of XPO4, FGF6, WDR49, ARMCX2, FSTL5, NRSN2, WDR37, ARMCX1, GJD4, ZBBX, GLO1, DDX20, SET, and BTBD9, or a fragment, variant, or analog thereof, wherein the protein is fused to a tumor targeting moiety, such as a tumor targeting moiety that binds to a cancer cell, such as a liver cancer cell or a breast cancer cell. In one embodiment, the tumor targeting moiety is an antibody that binds to a cancer cell, such as a liver cancer cell or a breast cancer cell.

In another embodiment, the invention provides compositions, such as pharmaceutical compositions, that can be used to inhibit proliferation of a cell, such a cancer cell, for example a liver cancer cell or a breast cancer cell, wherein composition comprises an agent that inhibits the expression and/or activity of the EIF5A2 gene or the EIF5A2 gene product, including, but not limited to, interfering RNA molecules that target EIF5A2, such as an shRNA or an siRNA molecules.

In other aspects, the present invention provides combination compositons that comprise combinations of one or more of the agents listed in the above paragraphs, and/or that comprise one or more of the agents listed in the above paragraphs in combination with one or more other agents useful for inhibiting cell proliferation and/or for treating cancer, including, but not limited to, chemotherapeutic agents and the like.

In another aspect, the present invention provides a pharmaceutical composition for the treatment of cancer in which the activity of said tumor suppressor is less than in normal tissue, comprising a tumor suppressor protein or a physiologically active fragment, analog, or mutant thereof. Another particular embodiment is a pharmaceutical composition for the treatment of cancer in which the activity of a tumor suppressor is less than in normal tissue, comprising a tumor suppressor gene or a fragment or mutant thereof that encodes a physiologically active polypeptide is introduced into the cancer tissue and expressed. In yet another embodiment, a pharmaceutical composition comprises one or more therapeutic agents that modulate known upstream factors of an identified tumor suppressor to increase the tumor suppressor expression.

More particularly, an embodiment of this aspect of the invention can be practiced using the tumor suppressor genes listed herein, or any other genes that are identified using the screening method described herein. More particularly, said tumor suppressor gene can be selected from XPO4, FGF6, WDR49, ARMCX2, FSTL5, NRSN2, WDR37, ARMCX1, GJD4, ZBBX, GLO1, DDX20, SET, and BTBD9.

In further aspects, the present invention provides methods for screening for therapeutic agents. Cells with deactivated or knocked down tumor suppressor expression or activity are useful in vitro and in situ for screening candidate therapeutic agents. Therefore, the identified shRNAs targeting tumor suppressors are useful in such screening therapeutic assays. In addition, shRNAs that target factors downstream of identified tumor suppressors can also be used in such screening methods. Moreover, in one embodiment, cells having reduced tumor suppressor expression or loss of tumor suppressor function, and non-human animals implanted with such cells, can be used to identify therapeutic agents that modulate downstream substrates or signaling pathway components for that specific tumor suppressor. For example, a cell in which XPO4 is downregulated or deactivated can be used in vitro and in situ to evaluate potential therapeutic agents that target the XPO4 substrates SMAD3 and EIF5A1.

One aspect of the invention is a method for testing a tumor, such as a lymphoma arising from an Eu-myc/shRNA tumor suppressor-transformed lymphoma, or a tumor arising from a tumor suppressor-transformed embryonic hepatocytes, for sensitivity to a treatment. Tumor cells, e.g. lymphoma cells, can be cultured in vitro, the cells are contacted with a candidate treatment and monitored for growth (e.g., by observing cell number, confluence in flasks, staining to distinguish viable from nonviable cells). Failure to increase in viable cell number, slower rate of increase in cell number, or a decline in viable cell number, compared to cells which are untreated or mock-treated, is an indication of sensitivity to the treatment.

The treatment to be tested can be one or more substances, for example, a known anti-cancer agent, such as adriamycin, cylophosphamide, prednisone, vincristine or a radioactive source. The treatment can also be exposure to various kinds of energy or particles, such as gamma-irradiation, or can be a combination of approaches. In some cases, the treatment can also be administration of one or more substances or exposure to conditions, or a combination of both, wherein the effects of the treatment as anti-cancer therapy are unknown. In addition, candidate agents can be selected based on their effect on downstream substrates or modulators of specific tumor suppressors.

Candidate agents may be further tested in tumors in situ in a mouse. For example, animals can be tested essentially as described in U.S. Pat. No. 6,583,333. Briefly, Eu-myc transgenic mice are treated with maximum tolerated dose of a candidate therapeutic agent (for example, 10 mg/kg body weight) by intraperitoneal injection. Treated mice are monitored for remission and for relapse by palpation and by blood smears to obtain white blood cell counts. Palpation is performed by gently feeling the mouse for bumps under the skin, which are enlarged lymph nodes. Blood smears are done by collecting blood from the tail artery, and examining a dried droplet of blood which has been smeared on a glass microscope slide to be one cell layer thick at the edge. The blood smear is stained after drying, using LEUKOSTAT™ stain (Fisher Diagnostics cat. #CS43A-C). The blood smear can be mounted with Permount™ histological mounting medium (Fisher Scientific). Slides are viewed under 40× or oil emersion. Blood from mice affected by lymphoma are always compared with blood from mice from a normal mouse drawn at the same time.

The invention further relates to a hepatocyte transfected with an RNAi molecule, wherein the expression of the RNAi molecule transforms the hepatocyte into a cancer cell. In certain embodiments, the hepatocyte is a c-myc/p53−/− embryonic hepatocyte.

The invention further relates a non-human animal model comprising: (1) a hepatocyte transfected with an RNAi molecule, wherein the expression of the RNAi molecule causes the animal to develops liver cancer.

### BRIEF DESCRIPTION OF THE DRAWINGS

FIGS. 1A-1E show development and characterization of a new orthotopic, genetically tractable mouse model for hepatocellular carcinoma. (1A) Schematic outline of two claims of producing a non-human animal liver cancer model of this invention. E-Cadherin+ mouse hepatoblasts are isolated from day 13-15 mouse liver using the MACS® indirect labeling system in combination with the ECCD-1 E-Cadherin antibody. Purified hepatoblasts are grown in short term primary-culture on irradiated NIH-3T3 feeder layers. The hepatoblasts are infected with GFP-tagged murine stem cell virus (MSCV) based retroviruses expressing oncogenes of interest (e.g., the H-ras oncogene) and/or expression cassettes for short hairpin RNAs directed against tumor suppressor genes (e.g., p53). After viral transduction, infected hepatoblasts are either injected into the spleens of retrorsine-conditioned recipient mice or subcutaneously into NCR nu/nu mice. Retrorsine efficiently blocks the cell cycle of hepatocytes and additionally causes a moderate liver damage by triggering apoptosis in a small number of hepatoblasts. Using this approach, after intrasplenic transplantation, genetically modified hepatocytes migrate via the portal vein into the recipient liver and engraft the organ. Transplanted hepatoblasts harboring the defined genetic lesions clonally expand and hepatocellular carcinomas develop in the liver. Tumor onset and growth kinetics can be monitored by external whole body GFP-imaging as all viral vectors carry a GFP expression cassette. (1B) Transplanted hepatoblasts engraft the recipient liver and are morphologically indistinguishable from the host hepatocytes (H&E). Immunofluorescence with a primary antibody directed against GFP allows detection of the transplanted hepatocytes (middle). DAPI counterstaining (right). (1C) p53 deficient liver progenitor cells transduced with different oncogenes (myc, akt or H-rasV 12) give rise to orthotopic liver carcinomas after intrahepatic seeding. Detection of intrahepatic liver carcinomas by whole body, external GFP-tumor imaging (top panel) or direct imaging of the respective explanted tumor bearing livers (bottom panel).Tumors can be detected by either external GFP imaging (upper panel) or direct GFP-imaging of the explanted liver (lower panel). (1D) Kaplan-Meier curve for survival times of mice transduced with different oncogenes (myc, akt, H-rasV12). All groups succumb to death much earlier than mice injected with p53−/− control vector alone. (1E) Cells from primary murine liver carcinomas (p53−/−; myc) were grown briefly in culture. In situ liver carcinomas were generated by direct subcapsular injection of the cells into the left liver lobe. Shown are GFP-tumor imaging (left) and a photograph of the in situ tumors (right) day 42 post-injection.

FIGS. 2A-2E show genome-wide analysis of copy number alterations in mouse hepatocellular carcinoma (HCC). DNA from tumors and subjected to 85K ROMA. Plotted is the normalized log-ratio for each oligo probe and ordered according to genome position, derived from the May 2004 freeze of the draft mouse genome sequence, available on the University of California Santa Clara genome website. (2a) Representative profiles of 3 mouse HCCs. HCC-7 and HCC-9, both derived from p53−/−; c-myc hepatoblasts, contain an amplification on chromosome 9. HCC-11, derived from p53−/−; Akt hepatoblasts, does not. (2b) Expanded view of chromosome 9 reveals a 1.9 Mb amplicon (HCC-9) and a 1.2 Mb (HCC-7) amplicon containing the c-IAP-1 and c-IAP-2 genes. (2c) Quantitative PCR with primers specific for the c-IAP-1 gene revealed higher copy numbers for 2 additional p53−/−; c-myc HCCs (HCC-13 and HCC-14), while non-c-myc tumors (HCC-15 and HCC-17) have a normal IAP copy number. (2d) Summary of c-IAP-1/2 amplification relative to genetic background. (2e) c-IAP-1 and c-IAP-2 mRNA levels are elevated in tumors containing the amplicon. Levels of IAP RNA relative to actin were determined by quantitative RT-PCR and normalized to normal liver.

FIGS. 3A-3D show genome-wide analysis of a human hepatocellular carcinoma analyzed with 36K ROMA. (3a) The three peaks indicate amplicons containing the MET-oncogene, Cyclin D and c-IAP1/2 (left to right). (3b) Expanded view of chromosome 11 showing the amplicons containing cyclin D and c-IAP1/2. (3c) 1/25 human HCCs have elevated c-IAP-1 and c-IAP-2 gene copy numbers as determined by quantitative PCR off genomic DNA. (3d) c-IAP-1 and/or c-IAP-2 mRNA levels are elevated in 4/25 HCCs as determined by quantitative RT-PCR.

FIGS. 4A-4E show that c-IAP-1 overexpression accelerates tumor growth. (4a) Schema of the approach to investigate the oncogenic properties of c-IAP-1 in vivo. E-Cadherin+ hepatoblasts were either double-infected with c-myc+control vector or c-myc+myc-tag-c-IAP-1. 10×106 cells were subcutaneously injected into irradiated NCR nu/nu mice. (4b) Overexpression of c-IAP-1 in primary liver cells was confirmed by western blot analysis using an a -myc-tag antibody. (4c) Four out of six c-myc+c-IAP-1 double infected tumors show accelerated tumor growth compared to c-myc+vector. Tumor size was assessed by caliper measurement of subcutaneously growing tumors. (4d) All tumors showing accelerated growth contain the c-IAP-1 provirus as assayed by PCR. All analyzed tumors contain c-myc-provirus DNA. (4e) Representative example of an accelerated c-IAP1+c-myc double infected tumor (left) compared to a c-myc+vector infected tumor (right). External GFP-imaging of the tumors (bottom) was performed at the same time post-injection.

FIGS. 5A-5D show that suppression of c-IAP-1 in HCC cells slows tumor growth. (5a) Schema for testing knock-down of c-IAP-1 expression in vivo. Cells from tumors containing the c-IAP1/2 amplicon are outgrown briefly and infected either with a retrovirus expressing a short hairpin (miR30 design) RNA directed against c-IAP-1 or with control vector. After puromycin-selection, cells were injected subcutaneously into NCR nu/nu mice. (5b) One out of four short hairpins directed against c-IAP-1 suppresses c-IAP-1 expression. NIH 3T3 cells were transiently transfected with pcDNA-myc tag-c-IAP-1 together with the respective hairpin. Western blot was performed using an a-myc-tag antibody. c-IAP-1-hairpin “1477” shows >95% knockdown. (5c) Tumors with stable RNAi mediated knockdown of c-IAP-1 show decelerated tumor growth compared to control vector infected tumors. Growth of subcutaneous tumors was assesed by caliper measurement. (5d) Representative example of a slower growing tumor with c-IAP-1 knockdown (right) compared to a control vector infected tumor. External GFP-imaging of the tumors (top panel) was done at the same time post-injection.

FIGS. 6A-6C show the influence of c-IAP-1 overexpression on proliferation and apoptosis in cultured hepatoblasts. (6a) ECadherin+hepatoblasts were infected with a neomycin selectable retrovirus overexpressing c-myc and a puromycin selectable retrovirus overexpressing c-IAP-1 or control vector. After neomycin/puromycin selection cells were plated at 4.5×10³ cells/cm² and growth rate was assessed by daily counting of the total cell number. c-IAP-1 overexpressing cells have a slight growth advantage. (6b and 6c) c-myc+c-IAP-1 or c-myc+vector double infected hepatoblasts.

FIG. 7 shows an example of subcutaneous liver cancer model of this invention on the genetic constellation p53−/−+Akt-over expression and its uses in evaluating tumor therapy. Akt is an apoptotic regulator that is activated in many cancers and may promote drug resistance in vitro (Mayo et al., “PTEN protects p53 from Mdm2 and sensitizes cancer cells to chemotherapy,” J. Biol. Chem. 277: 5484-5489 (2002)). The graph shows that the tumor's intrinsic chemoresistance against the cancer drug Gemcitabine® (brand name “Gemzar” in the Figure) can be reversed by application of a downstream effector of Akt, the mTOR (mammalian target of rapamycin) inhibitor Rapamycin.

FIGS. 8A-8D show that ROMA identifies localized DNA amplifications in murine HCCs. (8a) Genome-wide profile of a tumor derived from p53−/−; ras embryonic hepatoblasts reveals 2 amplifications on chromosome 15. Data plotted is the normalized log ratio for each probe (85K), sorted by chromosome position, of copy number for tumors relative to a normal reference DNA. (8b) Single probe resolution of chromosome 15 reveals increased copy number for Rnf19 and myc. (8c) Genome-wide profiles of three independent HCCs (Tu-7, Tu-9, Tu-13) derived from p53−/−; myc embryonic hepatoblasts are overlaid and reveal a recurrent overlapping DNA amplification on chromosome 9. (8d) Single probe resolution of the amplicon on chromosome 9qA1; minimal overlap region contains genes indicated.

FIGS. 9A-9E show that ROMA identifies amplification of the human syntenic region 11q22 in HCC and other cancers. (9a) Genome-wide profile of a human HCC reveals an amplification on chromosome 7 containing the c-MET gene and 3 regions amplified on chromosome 11. (9b) Single probe resolution of chromosome 11; 11q13 contains CCND1; B′ contains no known genes; 11q22 contains the genes depicted. (9c) Single probe resolution of chromosome 11 of a representative esophageal tumor. 11 q22 contains same genes as shown in b. (9d) Genome-wide profile of an ovarian carcinoma reveals chromosome 11 amplification. (9e) Single probe resolution of the 11q22 amplicon shows a lack of amplification of the MMP cluster.

FIGS. 10A-10D show that clAP1 is overexpressed in tumors containing elevated clAP1 gene copy number. (10a) clAP1 and c1AP2 are overexpressed in murine HCCs as determined by quantitative realtime RT-PCR analysis. *denotes tumors with elevated clAP1 gene copy number. (10b) clAP1 protein is overexpressed in outgrown murine HCC tumor cells containing the 9qA1 amplicon as assayed by immunoblotting using a monoclonal anti-clAP antibody (top panel) or a polyclonal anti-clAP1/2 antiserum (middle panel). Tubulin served as a loading control. (10c) Quantitative real-time RT-PCR analysis of clAP1 and c1AP2 expression in human HCCs. (10d) Quantitative real-time RT-PCR analysis of clAP1 and clAP2 expression in a subset of human esophageal carcinomas. The * in 10c and 10d denotes tumor with elevated clAP1 gene copy number.

FIGS. 11A-11E show that clAP1 overexpression in p53−/− myc hepatoblasts suppresses p53 independent forms of apoptosis induced by different death stimuli. (11a) Expression of myc-tagged-c1AP1 in p53−/− myc liver progenitor cells (right lane) was confirmed by western blot analysis using a monoclonal anti-clAP1 antibody. Left lane (V) is lysate from cells infected with vector alone. (11b) clAP1 expression protects hepatoblasts from apoptosis mediated by serum withdrawal. p53−/− hepatoblasts, double infected with myc+clAP1 or myc+vector were grown in the serum conditions indicated for 48 hrs. Apoptosis was measured using the Cell Death Detection ELISAPLUS (Roche). (11c) clAP1 protects against spontaneous cell death mediated by contact inhibition. p53−/− hepatoblasts (myc+clAP1 or myc+vector) were grown to confluence and apoptosis was measured as in 11b, 24 hours later. (11d) clAP1 protects against FasL triggered cell death but not TNFa or TRAIL mediated cell death of liver progenitor cells. p53−/− hepatoblasts (myc+clAP1 or myc+vector) were treated with 125 ng/ml TRAIL, 5 ng/ml TNFa or increasing concentrations of FasL (25, 50, 100 ng/ml) together with 2.5 ug/ml cycloheximide for 12 hrs. Apoptosis was measured as in 11b. Error bars in 11b-11d denote the standard deviation of three measurements per data point. (11e) clAP1 increases short and long-term viability following FasL treatment. p53−/− hepatoblasts (Myc+c1AP1 or Myc+vector) were treated with 50 ng/ml FasL for 36 hrs. Representative phase contrast photographs (upper panel, left) depict floating, apoptotic cells (arrow) and viable, attached cells (right, arrowhead). Cells that underwent the same treatment were cultured for 5 more days without FasL and stained with crystal violet to visualize clonogenic survival (lower panel).

FIGS. 12A-12F show that clAP1 enhances the tumorigenicity of Myc overexpressing p53−/− hepatoblasts. (12a) p53−/− hepatoblasts were double-infected with myc plus myc-tagged-clAP1 or myc plus vector and were subcutaneously injected into the rear flanks of nude mice (n=6 for each group). Tumor size was assesed by caliper measurement. Shown is a representative of three independent experiments. (12b) Immunoblot of tumor lysates for clAP1 protein level. Protein lysates from 6 representative tumors overexpressing myc-tagged-c1AP1 (lanes 8-13) and control vector tumors (lanes 5-7) were probed with anti-clAP1 antibody. Cells lysates from cultured myc-tagged-c1AP1 expressing hepatoblasts (M, lane 2) or vector alone (V, lane 1) and from 9qA1 amplicon containing cells (A+, lane 4) were loaded for comparison to indicate exogenously expressed, myc-tagged-clAP1 (75kD) and endogenous c1AP1 (65kD). A− is lysate from amplicon negative cells of the same genotype (p53−/−; myc). Tubulin is used as a loading control. (12c) clAP1 does not enhance the tumorigenicity of H-rasV12 overexpressing p53−/− hepatoblasts. n=6 for each group. (12d) Immunoblot of tumor lysates from 3 representative mice of each group shown in 12c probed with anti-clAP1 antibody. (12e) clAP1 does not enhance the tumorigenicity of Akt overexpressing p53−/− hepatoblasts. (12f) Immunoblot of tumor lysates from 3 representative mice of each group shown in 12e probed with anti-clAP1 antibody.

FIGS. 13A-13D show that tumors bearing the 9qA1 amplicon show delayed growth upon c1AP1 and c1AP2 suppression. (13a) Hepatoma cells outgrown from a 9qA1 amplicon positive, p53−/−; Myc tumor, were double infected with shRNAs targeting clAP1 and c1AP2 or control vectors (V), or no vector (−). Expression of clAP1 and c1AP2 is significantly reduced as shown on the immunoblots that were probed with a monoclonal anti-clAP1 antibody (top panel) and a polyclonal anti-clAP1/2 antibody (second panel). The levels of XIAP were not reduced (third panel). Equal amounts of protein were loaded as assessed by tubulin levels. The * denotes a non-specific band. (13b) Stable suppression of c1AP1 and clAP2 slows tumor growth of p53−/−; myc mouse hepatoma cells that contain the 9qA1 amplicon. Tumorigenicity of the cells described in 13a after injection into the rear flanks of nude mice. Growth of subcutaneous tumors was assessed by caliper measurement. (13c) Stable suppression of clAP1 and clAP2 does not slow tumor growth of p53−/− myc mouse hepatoma cells that do not contain the 9qA1 amplicon. (13d) Stable suppression of p53 does not slow tumor growth of p53−/− myc mouse hepatoma cells that contain the 9qA1 amplicon.

FIGS. 14A-14C show in vivo RNAi screening to identify new tumor suppressor genes in liver cancer. (14a) Schematic illustration of an in vivo RNAi screening protocol according to the teachings of the invention. (14b) and (14c) Shuffling of SalI/MluI fragments (containing shRNAmir and a unique barcode sequence for every shRNA) from the low complexity pSM2c library pools (14b) into an MSCV based retroviral vector which has been optimized for in vivo use (14c).

FIGS. 15A-15E show that shRNAmir molecules targeting the APC tumor suppressor gene trigger tumor growth.

FIGS. 16A-16G show the identification of new tumor suppressor genes in liver cancer, using in vivo RNAi screening. shRNAmir molecules targeting PTEN trigger the growth of liver carcinomas.

FIGS. 17A-17E show the functional validation of candidate tumor suppressor genes.

FIGS. 18A-18H show that the loss of Xpo4 promotes tumorigenesis.

FIGS. 19A-19C show a ROMA deletion based RNA interference library. FIG. 19A shows a representative whole genome ROMA plot of a human HCC. The blue circles represent the averaged fluorescent ratio (tumor vs. normal) and the orange lines correspond to the value determined by copy number segmentation. Arrows denote the focal deletion highlighted in FIG. 19C. FIG. 19B shows deletion counts in ROMA profiles of 98 human HCC. A deletion count in each profile set was obtained by finding the maximal tier number for 24719 genes across the genome in each profile in the set and summing the result over the set. The counts were plotted against the absolute genomic position, with the genes sorted by their genomic transcription start position. Dashed lines denote chromosome boundaries. The points in the vicinity of 58 focal deletions (containing 362 genes) are highlighted by red circles. FIG. 19C shows a representative 524 Kb focal deletion on chromosome 12 contains ten genes.

FIGS. 20A-20B show generation of ROMA deletion-based shRNA library pools. FIG. 20A is a schematic representation of the workflow for compiling a murine shRNA library targeting genes found in recurrent focal deletions of 98 human HCCs. FIG. 20B shows a subcloning strategy for shuttling shRNA library pools from the pSM2 CODEX library vector into pLMS (MSCV-SV40-GFP).

FIGS. 21A-21E show the setup of in vivo RNAi screening. FIG. 21A is a schematic representation of the approach. ED=18 Trp53−/− liver progenitor cells were immortalized by transduction with a Myc expressing retrovirus. Subsequently, the cells were infected with single shRNAs or shRNA library pools and injected into the liver or subcutaneously, to allow tumor formation. FIG. 21B shows a growth curve of tumors derived from Trp53−/−; Myc cells infected with a control shRNA or three Apc shRNAs. Values are average of 6 tumors. The inset shows knockdown of Apc protein assayed by Western blot. FIG. 21C shows bioluminescence imaging of tumors derived from Trp53−/−; Myc cells infected with a control shRNA or Apc shRNA and transplanted into the livers of immunocompromised recipient mice (n=4). Animals were imaged 40 days post surgery. FIG. 21D shows H&E and β-catenin staining of liver tumors in FIG. 21C. Normal liver served as a control. FIG. 21E shows a tumor growth curve of Trp53−/−; Myc cell-derived tumors infected with control shRNA (control), Apc shRNA (shAPC), a 1:50 diluted Apc shRNA (shAPC 1:50), Axin shRNA and a shRNA library pool (pool A7EH, taken from the Cancer1000 library).

FIGS. 22A-22G shows that in vivo shRNA library screening identifies PTEN as a potent tumor suppressor in HCC. FIG. 22A shows the average volume (n=8) at indicated time points of tumors derived from Trp53−/−; Myc cells infected with a control shRNA (control) and ten random genome-wide shRNA pools (pool size n=48). FIG. 22B shows the average volume (n=8) of tumors derived from Trp53−/−; Myc cells infected with a control shRNA (control) and 13 ROMA deletion shRNA pools (pool size n=48). Red asterisks indicate tumors/shRNA pools subjected to subcloning and sequencing as shown in FIG. 22C. FIG. 22C shows a representation of the strategy to recover shRNAs from tumor genomic DNA by PCR and subcloning of the PCR products into the vector used for hairpin validation. FIG. 22D shows pie charts denoting enrichment of two Pten shRNAs in selected tumors (right) compared to their representation in pre-injection plasmid pools (left). Pie graphs show the representation of each Pten shRNA in the total shRNA population analyzed by high-throughput sequencing. FIG. 22E shows ROMA array CGH plot (Chromosome 10) showing a focal PTEN genomic deletion in a human HCC. FIG. 22F shows validation of the same Pten shRNAs using orthotopically transplanted Trp53−/−; Myc cells transformed with the Pten shRNAs. Representative imaging results from three mice in each group are shown. FIG. 22G shows shRNA mediated knockdown of Pten increases phospho-Akt. Protein lysates from Trp53−/−; Myc liver cells infected with Pten shRNAs (Cell) or the derived tumors (Tumor) were immunoblotted with the indicated antibodies. Tubulin served as a loading control.

FIGS. 23A-23D show that ROMA deletion shRNA library pools accelerate tumor formation. FIG. 23A shows bioluminescence imaging of immunocompromised mice injected with Trp53−/−; Myc cells transformed with a control shRNA or random shRNA pools taken from a genome-wide shRNA library. FIG. 23B shows a subcutaneaous tumor growth assay using the same cells as in FIG. 23A, transformed with a control shRNA (control) and 13 ROMA deletion shRNA pools. FIGS. 23C and 23D show the cut-off setup for the screen. Only the pools with a final average tumor volume≧0.1 cm³ (day 42, FIG. 23C) and a take rate≧50% (4 of 8 injected sites, FIG. 23D) were sequenced. Note that none of the controls fit these two criteria. The red lines denote cut-off threshold. Red asterisks indicate tumors/shRNA pools subjected for sequencing as shown in FIG. 22B.

FIG. 24 shows validation of two Pten shRNAs using Trp53−/−; Myc cells in a subcutaneous tumor growth assay. Error bars denote S.D. (n=4).

FIGS. 25A-25D show novel tumor suppressor genes identified by in vivo RNAi screening. FIG. 25A shows a representation of some scoring shRNAs in the tumors. The representation of each shRNA is ˜2% in the pre-injection plasmid pools, as shown in FIG. 22D. FIG. 25B shows ROMA array CGH plots depicting focal genomic deletions of the genes targeted by the enriched shRNAs as shown in FIG. 25A. FIG. 25C shows validation of the top scoring shRNAs in a subcutaneous tumor growth assay, as described in FIG. 22F (n=4). Asterisks depict genes that were further analyzed as shown in FIG. 25D. FIG. 25D shows in situ validation of at least three independent shRNAs targeting genes as depicted in FIG. 22C. Trp53−/− liver progenitor cells were transformed with Myc and the indicated shRNAs, and seeded into livers of recipient mice. Tumor growth was followed up by bioluminescence imaging. Asterisks indicate CODEX shRNAs that were initially identified in the screen. Additional shRNAs against each target were synthesized de novo. Representative bioluminescence imaging results from three mice are shown.

FIGS. 26A-26B show validation of shRNA-mediated knockdown of gene expression. FIG. 26A shows 293T cells co-transfected with a 6xMyc tagged XPO4 cDNA and XPO4 shRNAs. Protein lysates were immunoblotted with Myc tag antibody. FIG. 26B shows RT-Q-PCR of murine cells infected with the indicated shRNAs. Values for each gene are normalized to the control shRNA.

FIG. 27 shows validation of scoring candidates with multiple shRNAs. Subcutaneous tumor growth assays were performed as in FIG. 22F. Tumor volume over time is plotted. Values represent the average of four tumors.

FIGS. 28A-28B show validation of SET shRNAs in situ. FIG. 28A shows a ROMA array CGH plot depicting a focal SET deletion in a human HCC. The red arrow denotes the genomic position of SET. FIG. 28B shows bioluminescence imaging of mice injected with Trp53−/−; Myc cells transformed with a control shRNA and two Set shRNAs. Representative imaging results from three mice in each group are shown.

FIGS. 29A-29B show validation of scoring shRNA in an idependent population of Trp53−/−; Myc liver cells. In FIGS. 29A and 29B, scoring shRNAs were validated in an idependent Trp53−/−; Myc line, which was derived from another batch of embryonic hepatoblasts but had the same genetic background and pathological phenotype. The cells were transformed with the indicated shRNAs and subcutaneously injected into nude mice. Data shown are an average of four tumors in each group. An shRNA targeting Apc (shRNA.2243) serves as a positive control.

FIGS. 30A-30B show that shRNA-mediated knockdown of XPO4 deregulates TGF-β signaling in mouse liver cells. FIG. 30A shows Smad3 and phospho-Smad3 Western blots of nuclear (N) and cytoplasmic (C) fractions of Trp53−/−; Myc liver cells infected with a control shRNA and different Xpo4 shRNAs. Histone H3 (H3) and Mck1 serve as loading controls. FIG. 30B shows RT-Q-PCR analysis of TGF-f3 target genes in Trp53−/−; Myc liver cells with Xpo4 knockdown. Two independent hairpins were used to reduce Xpo4 expression. Cells infected with a control hairpin and stimulated with recombinant TGF-β served as a control.

FIGS. 31A-31I show reporter assay-based shRNA ranking. The figure shows a histogram for single variable readout for the evaluation of shRNA efficacy. Potent shRNAs receive a positive value (maximum 1) and inefficient shRNAs receive a negative value (minimum −1).

FIG. 32 shows that EIF5A2 is a key downstream effector of XPO4 in tumor suppression. FIG. 32A shows a ROMA array CGH plot of a human HCC showing an EIF5A2 containing amplicon on chromosome 3. FIG. 32B shows that EIF5A2 expression promotes tumor formation in Trp53−/−; Myc liver progenitor cells. Subcutaneous tumor growth assays were performed as in FIG. 21B. Error bars denote S.D. (n=4). FIG. 32C shows that knockdown of EIF5A2 attenuates proliferation of human hepatoma cells harboring XPO4 deletion. Cell numbers were measured by MTT assay in human hepatoma cell lines Huh7 (wild type), Alexander (EIF5A2 amplicon) and SK-Hep1 (XPO4 deletion) 48 hrs post siRNA transfection. Error bars denote S.D. (n=3). FIG. 32D shows a colony formation assay of SK-Hep1 cells five days post-transfection using the indicated single siRNA or combination.

FIG. 33 shows siRNA-mediated knockdown of EIF5A in SK-Hep1 cells. Knockdown efficiency of EIF5A1, EIF5A2, and SMAD3 by specific siRNAs was quantified by RT-Q-PCR 24 hours post-transfection. Erro bars denote S.D. (n=3).

FIGS. 34A-34F show frequent copy number alterations of XPO4 and EIF5A2 in human breast cancer. FIG. 34A shows that XPO4 is frequently deleted in human breast cancer. Shown are the deletion counts in ROMA profiles of 257 human breast cancers with the genes sorted by their genomic transcription start position. Blue dots represent the deletion frequency counts for each gene on chromosome 13. The dashed line points to XPO4. FIG. 34B shows that EIF5A2 is frequently amplified in human breast cancer. Amplification counts (as described in FIG. 34A) of chromosome 3 in human breast cancer. The dashed line points to EIF5A2. FIG. 34C shows a ROMA plot of a 3.3 Mb spanning XPO4 deletion in the human breast cancer cell line MDA-MB-330. FIG. 34D shows a ROMA plot of a 5.8 Mb spanning EIF5A2 amplicon in a human breast carcinoma. FIG. 34E shows the distribution of scoring genes from the in vivo RNAi screen in a deletion count plot (98 human HCCs) as described in FIG. 19B. Blue dots denote the deletion frequency count for each gene in the genome. Green dots depict 13 scoring genes (embedded in 11 focal deletions) from the HCC RNAi screen. Dashed lines represent chromosome boundaries. Note that ARMCX1 and ARMCX2, which are located on the X chromosome, are not included. FIG. 34F shows that new tumor suppressor genes identified through the HCC in vivo RNAi screen are also frequently deleted in human breast cancer. Green circles depict the newly identified tumor suppressor genes in a deletion count frequency plot of 257 human breast cancers.

### DETAILED DESCRIPTION OF THE INVENTION

### Definitions & Abbreviations

The terms “comprises,” “comprising,” “containing,” “having,” and the like, have the meaning ascribed to them in U.S. Patent law and mean “includes,” “including,” and the like.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “analog” is a molecule that can be a peptide or a structurally similar peptidomimetic, which has substantially similar biological activity to a given protein, e.g. a tumor suppressor protein or oncogenic protein of the invention. An analog can be a fragment of a full-length protein, a mutant having one or more deletions, insertions, or substitutions of amino acid residues within the polypeptide sequence, or a molecule composed partially or wholly of unnatural amino acids. An analog can also be a modified polypeptide having post translational modification, in vivo or in vitro.

An “antibody” is an immunoglobulin molecule comprising two heavy chains and two light chains, which recognizes an antigen. The immunoglobulin molecule may derive from any of the commonly known classes, including, but not limited to, IgA, secretory IgA, IgG, and IgM. IgG subclasses are also well-known to those skilled in the art and include, but are not limited to, human IgG1, IgG2, IgG3, and IgG4. It includes, by way of example, both naturally occurring and non-naturally occurring antibodies. Specifically, “antibody” includes polyclonal and monoclonal antibodies, and monovalent and divalent fragments thereof Furthermore, “antibody” includes chimeric antibodies, wholly synthetic antibodies, single chain antibodies, and fragments thereof Optionally, an antibody can be labeled with a detectable marker. Detectable markers include, for example, radioactive or fluorescent markers. Antibodies may also be modified by coupling them to other biologically or chemically functional moieties, such as cross-linking agents or peptides.

A “small molecule” is a compound having a molecular weight less than about 2500 amu, preferably less than about 2000 amu, even more preferably less than about 1500 amu, still more preferably less than about 1000 amu, or most preferably less than about 750 amu.

A “subject” can be a human or a non-human animal such as a mouse.

A “patient” is a human subject.

A “transformed cell” is one that has been genetically modified. Genetic modification can be stable or transient. Methods of transformation (i.e., introducing vectors or constructs into cells) include, but are not limited to, liposome fusion (transposomes), viral infection, and routine nucleic acid transfection methods such as electroporation, calcium phosphate precipitation and microinjection. Successful transformation will have an intended effect in the transformed cell, such as gene expression, gene silencing, enhancing a gene target, or triggering target physiological event.

In one embodiment, “treating” means slowing, stopping or reversing the progression of a disease or disorder. “Treating” can also mean amelioration of symptoms associated with a disease or disorder.

“Vector” refers to a vehicle for introducing a nucleic acid into a cell. Vectors include, but are not limited to, plasmids, phagemids, viruses, bacteria, and vehicles derived from viral or bacterial sources. A “plasmid” is a circular, double-stranded DNA molecule. A preferred type of vector for use in the present invention is a viral vector, wherein heterologous DNA sequences are inserted into a viral genome that may be modified to delete one or more viral genes or parts thereof. Certain vectors are capable of autonomous replication in a host cell (e.g., vectors having an origin of replication that functions in the host cell). Other vectors can be stably integrated into the genome of a host cell, and are thereby replicated along with the host genome.

The abbreviation “ROMA” refers to representational oligonucleotide microarray analysis.

The abbreviation “HCC” refers to hepatocellular carcinoma.

Further definitions and abbreviations are provided in context in the following description.

Unless otherwise defined herein, all other scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The methods and techniques of the present invention are generally performed according to conventional methods well known in the art and as described in various general and more specific references that are cited and discussed throughout the present specification, unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2003); Harlow and Lane, Antibodies: A Laboratory Manual, Cold Spring Harbor Laboratory Press, Cold Spring Harbor, N.Y. (1990); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All of the above and any other publications, patents and published patent applications referred to in this application are specifically incorporated by reference herein.

The genetically tractable, transplantable in situ liver or hepatocellular cancer models described herein offer unique advantages. The models employ the proliferative capacity of the liver to enable the altered hepatocytes to reconstitute liver tissue. Large amounts of primary epithelial cells can be isolated according to standardized protocols either from adult mouse livers or from embryonic mouse livers. The primary culture conditions for embryonic, as well as adult primary hepatocytes, are based on well-established protocols and are less complex compared to other epithelial primary cultures. A sample of the primary cells can be used for RT-PCR characterization for liver specific markers to rule out overgrowing by non-parenchymal cells.

Primary adult or embryonic hepatocyte cultures can be genetically modified by infection with lentiviral- or retroviral vectors carrying various genetic alterations, including oncogenes or short hairpin RNAs against tumor suppressor genes. Virally transduced primary hepatocytes can efficiently engraft the livers of non-human animals after transplantation into their portal vein or spleen. In the case of certain genetic configurations, mice developed hepatocellular carcinomas that could be visualized by whole body fluorescence imaging. For example, introduction of a myc retrovirus into p53 deficient hepatocytes produced highly aggressive tumors that show many features of human hepatocellular carcinoma. Overall, it provides rapid generation of genetically defined hepatocellular carcinomas.

In one aspect the invention embodies a method of making a non-human animal bearing a liver cancer using transplanted hepatocytes altered to increase oncogene expression, to reduce tumor suppressor gene expression or both. Preferably, the hepatocytes are virally transduced with a vector expressing an oncogene or a short hairpin RNA against a tumor suppressor gene and subsequently transplanted into a recipient non-human animal wherein the animal develops liver cancer tumors from at least one of the hepatocytes with altered gene expression.

As used herein, a non-human animal includes any animal, other than a human. Examples of such non-human animals include without limitation: aquatic animals, e.g., fish, sharks, dolphins and the like; farm animals, e.g., pigs, goats, cows, horses, rabbits and the like; rodents, e.g., rats, guinea pigs and mice; non-human primates, e.g., baboons, chimpanzees and monkeys; and domestic animals, e.g., cats and dogs. Rodents are preferred. Mice are more preferred.

The non-human animals can be wild type or can carry genetic alterations. For example, they may be immunocompromised or immunodeficient, e.g., a severe combined immunodeficiency (SCID) animal.

As used herein, hepatocytes include all descendants of embryonic liver progenitor cells. Preferably, primary hepatocytes are used in the methods and models of this invention. Primary hepatocytes from adult non-human animals or embryonic liver progenitor cells can be isolated using standard and conventional protocols. In short term primary culture the hepatocytes can be virally transduced with vectors carrying oncogenes and/or expression cassettes for short hairpin RNAs directed against tumor suppressor genes. Such transductions may be effected using standard and conventional protocols.

The term vector refers to a nucleic acid molecule capable of transporting another nucleic acid to which it has been linked. One type of vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in a host cell into which they are introduced (e.g., vectors having an origin of replication which functions in the host cell). Other vectors can be integrated stably into the genome of a host cell upon introduction into the host cell, and are thereby replicated along with the host genome. Preferred viral vectors include retroviral and lentiviral vectors. Moreover, certain preferred vectors are capable of directing the expression of nucleic acid sequences to which they are operatively linked. Such vectors are referred to herein as recombinant expression vectors or simply, expression vectors. Preferably, the vector carries marker cassettes, more preferably, GFP expression cassettes, so that the course of transduction, engrafting and tumor growth and remission may be observed. Preferably, the vector also carries a ubiquitous promoter to permit expression or up-regulation of oncogenes in all cell types of epithelium (i.e., stem cell and non-stem cell compartments).

As used herein, viral transduction refers to a general method of gene transfer. As embodied herein, viral transduction is used for establishing stable expression of genes in culture. Viral transduction and long-term expression of genes in cells, preferably cultured hepatocytes, is preferably accomplished using viral vectors.

After viral transduction the cells are preferably injected into the spleen of the recipient non-human animal, preferably a rodent and most preferably a mouse, that are preferably pretreated with a liver cell cycle inhibitor. Using this approach, the genetically modified or altered hepatocytes migrate via the portal vein into the recipient liver and engraft the organ. An additional proliferation stimulus to the liver can preferably be given after hepatocyte transplantation by serial administration of CCl₄.

Non-human animals harboring hepatocellular carcinomas of different genetic constellations produced by the altered hepatocytes can be characterized with regard to time to tumor onset and survival time. Tumors of different genetic constellations can also be histologically examined and classified by experienced pathologists.

As used herein, an altered hepatocyte refers to a change in the level of a gene and/or gene product with respect to any one of its measurable activities in a hepatocyte (e.g., the function which it performs and the way in which it does so, including chemical or structural differences and/or differences in binding or association with other factors). An altered hepatocyte may be effected by one or more structural changes to the nucleic acid or polypeptide sequence, a chemical modification, an altered association with itself or another cellular component or an altered subcellular localization. Preferably, an altered hepatocyte may have “activated” or “increased” expression of an oncogene, “repressed” or “decreased” expression of a tumor suppressor gene or both.

The increased expression of an oncogene refers to a produced level of transcription and/or translation of a nucleic acid or protein product encoded by an oncogenic sequence in a cell. Increased expression or up regulation of an oncogene can be non-regulated (i.e., a constitutive “on” signal) or regulated (i.e., the “on” signal is induced or repressed by another signal or molecule within the cell). An activated oncogene can result from, e.g., over expression of an encoding nucleic acid, an altered structure (e.g., primary amino acid changes or post-transcriptional modifications such as phosphorylation) which causes higher levels of activity, a modification which causes higher levels of activity through association with other molecules in the cell (e.g., attachment of a targeting domain) and the like.

The decreased expression of a tumor suppressor gene refers to an inhibited, inactivated or down regulated level of transcription and/or translation of a nucleic acid or protein product encoded by a tumor suppressor gene sequence in a cell. Reduced expression of a tumor suppressor gene can be non-regulated (i.e., a constitutive “off” signal) or regulated (i.e., the “off” signal is activated or repressed by another signal or molecule within the cell). As preferred herein, a repressed tumor suppressor gene can result from inhibited expression of an encoding nucleic acid (e.g., most preferably a short hairpin RNA using RNA interference approaches, see supra). Reduced expression of a tumor suppressor gene can also result from an altered structure (e.g., primary amino acid changes or post-transcriptional modifications such as phosphorylation) which causes reduced levels of activity, a modification which causes reduced levels of activity through association with other molecules in the cell (e.g., binding proteins which inhibit activity or sequestration) and the like.

As used herein the term liver or hepatocellular cancer or tumor refers to a group of cells which are committed to a hepatocellular lineage and which exhibit an altered growth phenotype. The term encompasses tumors that are associated with hepatocellular malignancy (i.e., HCC) as well as with pre-malignant conditions such as hepatoproliferative and hepatocellular hyperplasia and hepatocellular adenoma, which include proliferative lesions that are perceived to be secondary responses to degenerative changes in the liver.

The non-human animals of the invention are useful in the study of the impact of genotype on pathology or treatment response in vivo. Thus, the methods and models of the invention have implications for understanding disease progression in human liver carcinomas of specific genetic origin. The invention is also useful for determining the efficacy of a therapy in treating liver cancer. For example, a potential therapy may be administered to a non-human animal, produced by the methods embodied herein, and the non-human animal monitored for liver tumor formation, growth, progression or remission. Often, increased time to tumor formation or growth indicates sensitivity of the tumor to the therapy.

Genomic analysis of human carcinomas can be performed by gene expression profiling, e.g., ROMA. Such analysis in the tumors produced according to the invention has revealed a low signal to noise ratio of profiled genes, suggesting that the majority of detected genetic alterations in human tumors (having a high signal to noise ratio) may not be originally involved in tumor development but may be a by-product of tumor development. The analysis of mouse tumors produced according to the invention has shown that these tumors have a low signal to noise ratio, suggesting that a higher proportion of the identified lesions are specifically involved in tumor initiation/progression. Thus, the analysis of mouse tumors by gene expression profiling can serve as a filter for the “noisy” human tumors. Results obtained from mouse profiling using ROMA can be aligned with ROMA data obtained from human hepatocellular carcinomas. Overlapping amplifications or deletions then can be prioritized for further evaluation.

Tumors showing specific amplifications of candidate oncogenes in gene expression profiles can be outgrown in culture. Using stable RNAi, efficient knockdown of these genes can be achieved. Tumor cells with stable knockdown of a previously amplified gene can be re-transplanted into the mouse model of the current invention. Using this approach new therapeutic targets for hepatocellular carcinoma and related carcinomas can be obtained and the specific consequences of knocking down an amplified gene with regard to tumor growth or metastases can be studied. Drug therapies that specifically inhibit the identified targets can be developed.

Therapies that may be tested and evaluated in the methods and models of this invention include both general and targeted therapies. As used herein, a general therapy can be, for example, a pharmaceutical or chemical with physiological effects, such as pharmaceuticals that have been used in chemotherapy for cancer. Chemotherapeutic agents inhibit proliferation of tumor cells, and generally interfere with DNA replication or cellular metabolism. See, e.g., The McGraw-Hill Dictionary of Chemical Terms (Parker, S., Ed., McGraw-Hill, San Francisco (1985)). Chemotherapeutic agents may or may not have been characterized for their target of action in cells. However, this invention and its methods and models allow evaluation of such therapies for defined genetic alterations.

A targeted therapy refers to a therapy that directly interferes with a specific gene Preferably, a targeted therapy directly interferes with the expression of a gene involved in liver cancer. The effectiveness of a targeted therapy can be determined by the ability of the therapy to inhibit an oncogene or activate a tumor suppressor gene.

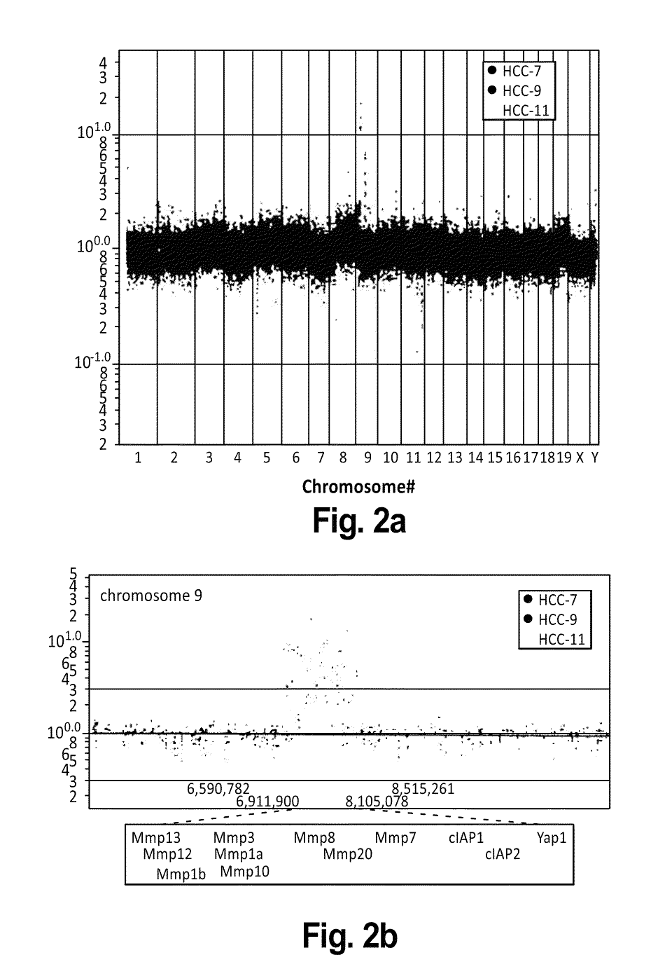

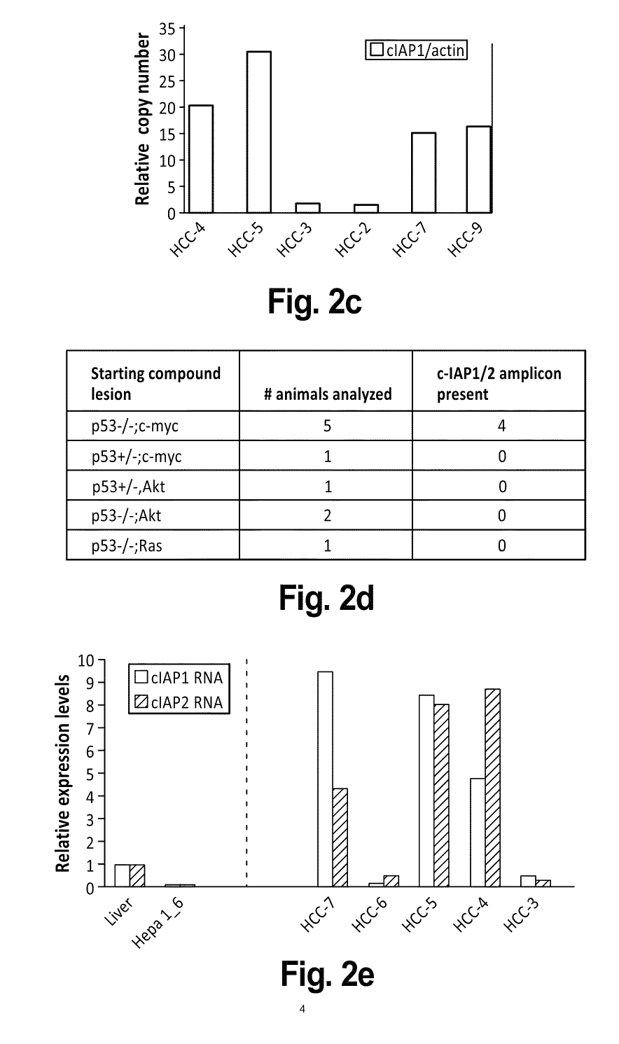

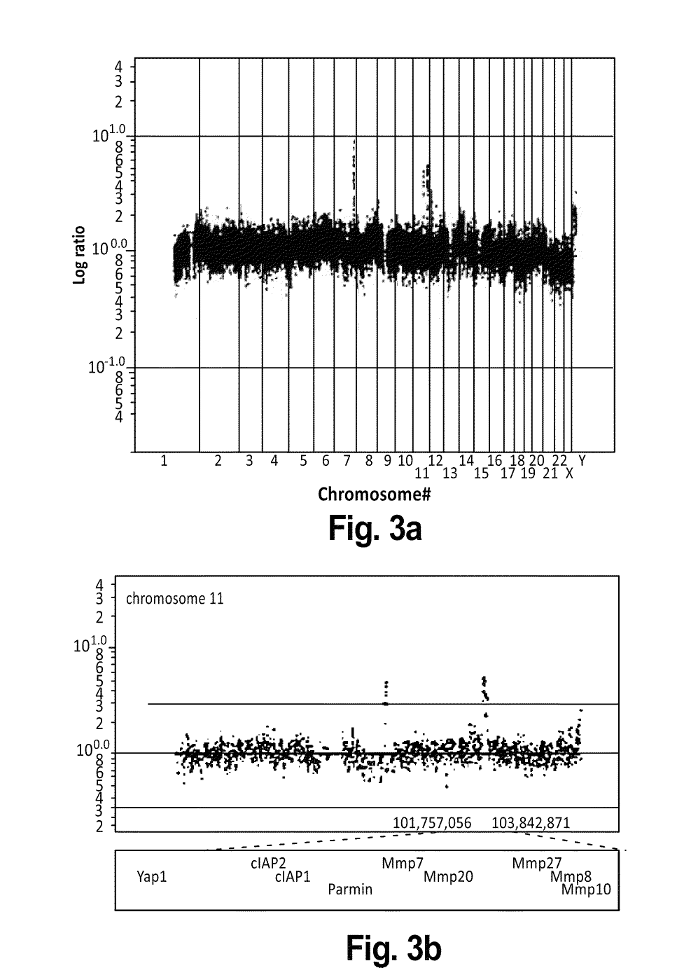

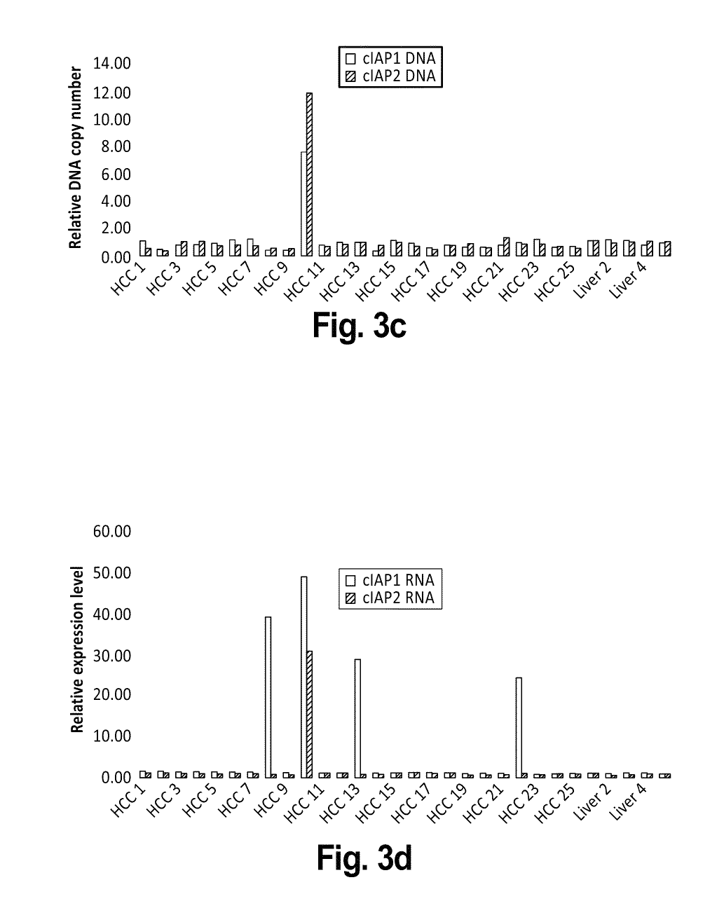

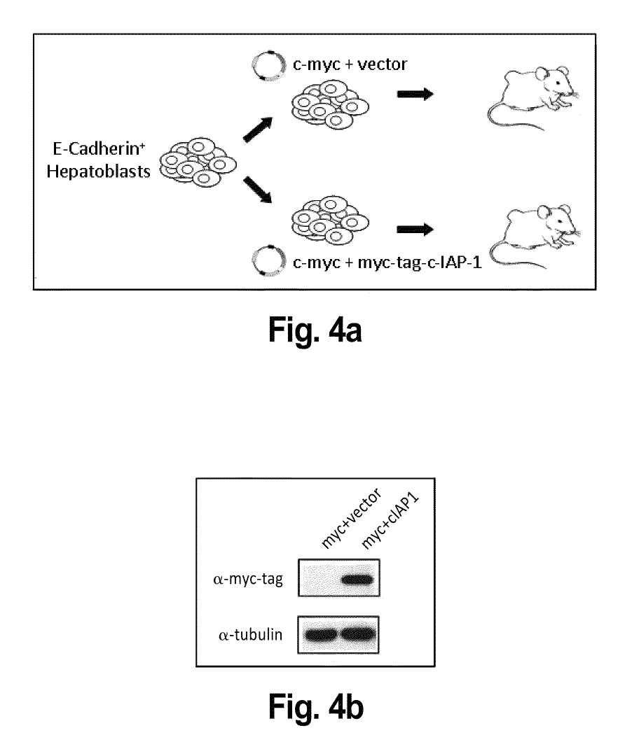

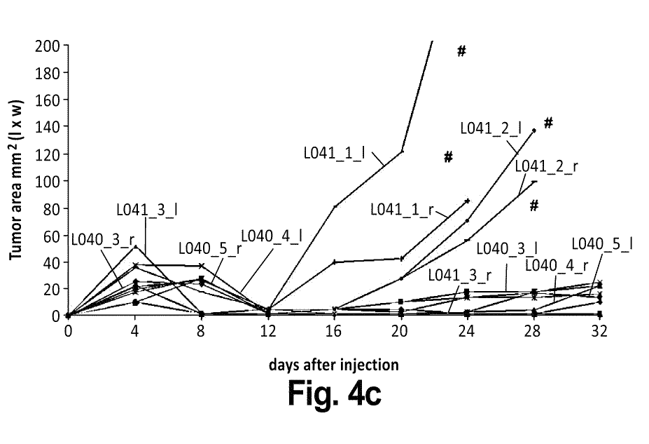

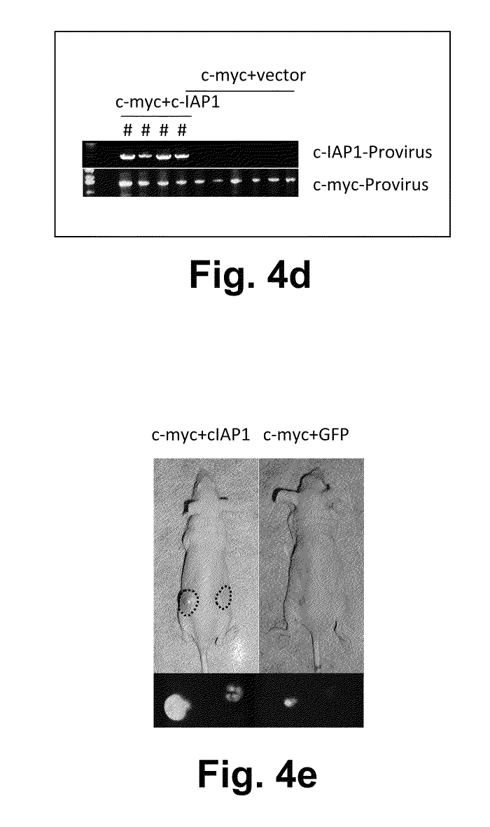

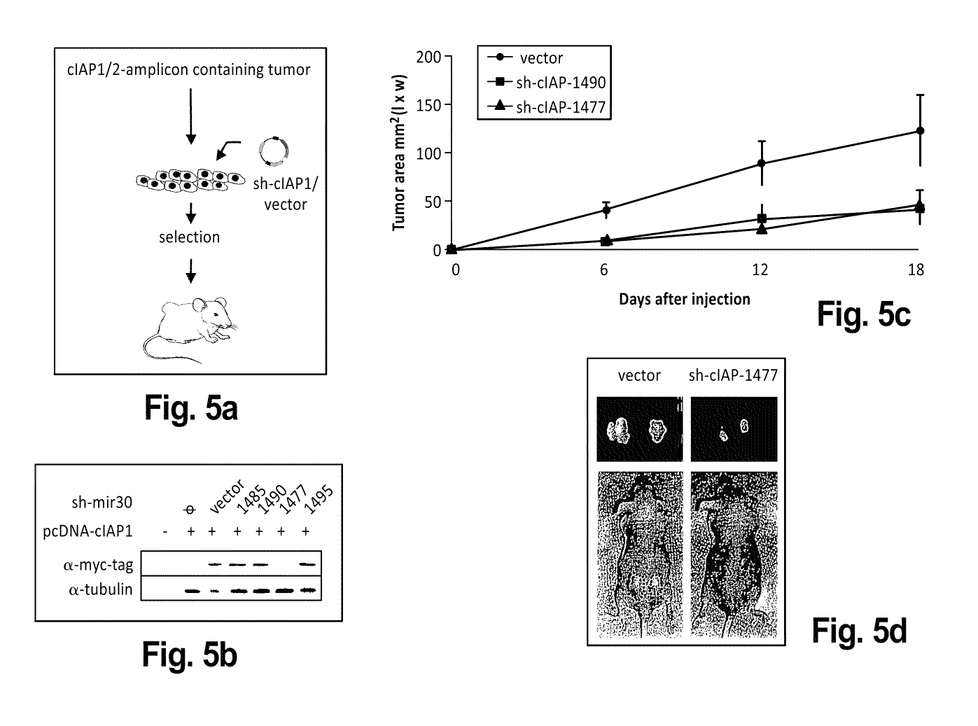

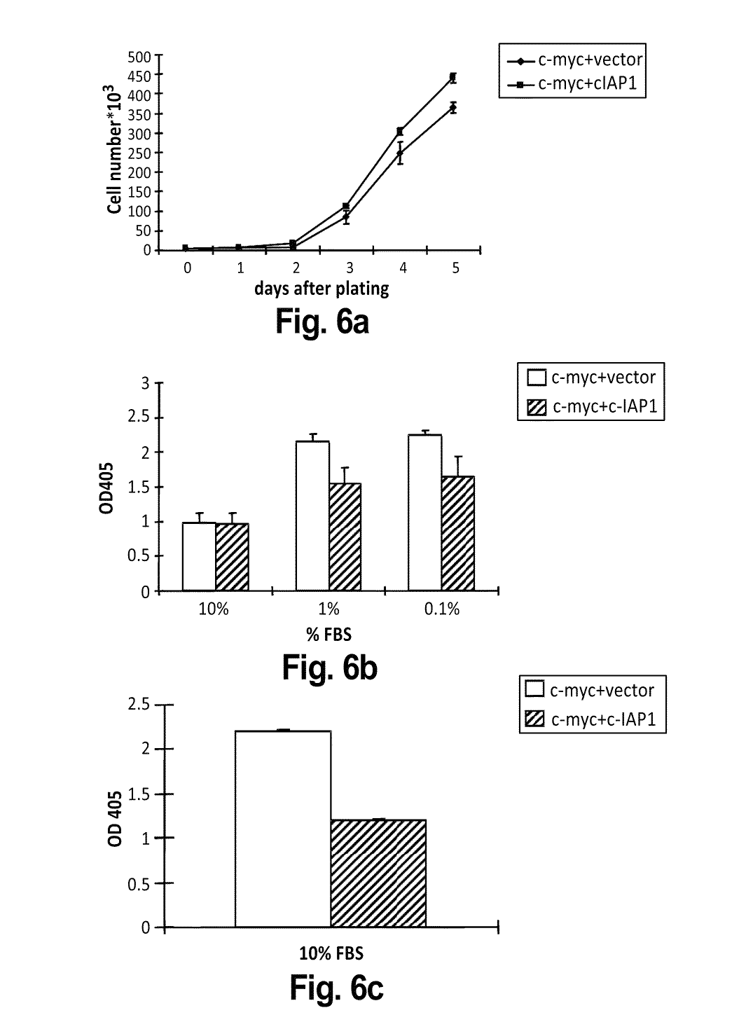

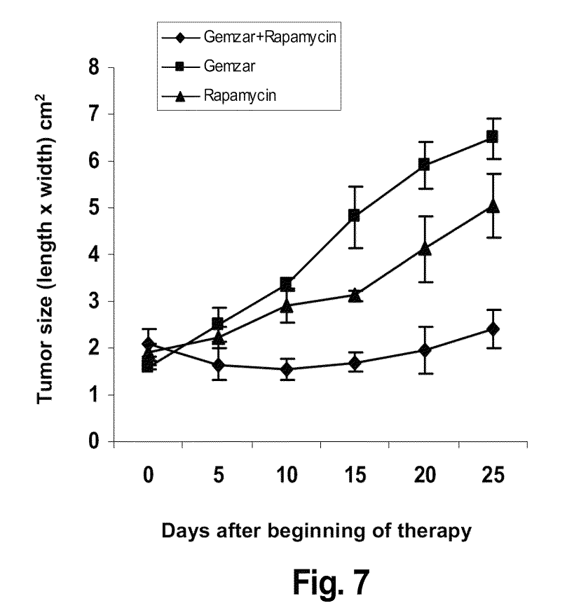

Most preferably, the therapies are used and evaluated in combination. For example, as shown in FIG. 7, upon onset of liver cancer tumors, animals can be treated with Gemcitabine (“Gemzar”), a chemotherapeutic agent that is an antimetabolite that functions as a mild chemotherapeutic to interfere with the growth of cancer cells. As shown in FIG. 7, it has virtually no effect on tumor growth of the particular tumor tested in FIG. 7. The tumor embodied in FIG. 2 can also be treated with Rapamycin, a targeted therapy that inhibits the mammalian target of rapamycin (mTOR). It has some effect on the tumor growth. In combination, however, as depicted in FIG. 7, the two therapies control tumor growth.

The size and growth of tumors after therapy can be monitored by a wide variety of ways known in the art. Preferably, whole body fluorescence imaging is used because the preferred viral vectors of this invention carry a GFP expression cassette. See, e.g, Schmitt et al., “Dissecting p53 tumor suppressor functions in vivo,” Cancer Cell 1:289-98 (2002). Tumors can also be examined histologically. Paraffin embedded tumor sections can be used to perform immunohistochemistry for cytokeratins and ki-67 as well as TUNEL-staining The apoptotic rate of hepatocytes can be analyzed by TUNEL assay according to published protocols. Di Cristofano et al., “Pten and p27KIP1 cooperate in prostate cancer tumor suppression in the mouse,” Nature Genetics, 27:222-224 (2001).

Beyond having important implications for understanding liver cancer, the evaluations and observations made possible by the methods and models of this invention provide insight into the utility of targeted approaches in cancer therapy.

In certain aspects the present invention uses RNA interference or “RNAi” and methodologies to identify tumor suppressor genes and oncogenes, and also uses RNAi methodologies to inhibit expression or achieve knockdown of certain genes, such as tumor suppressor genes and oncogenes.

RNAi is a powerful tool for in vitro and in vivo studies of gene function and for therapy in both human and veterinary contexts. RNAi is a sequence-specific post-transcriptional gene silencing mechanism triggered by double-stranded RNA. It causes degradation of mRNAs homologous in sequence to the double-stranded RNA. The mediators of the degradation are 21-23-nucleotide siRNAs generated by cleavage of longer dsRNAs by DICER, a ribonuclease III-like protein. Molecules of siRNA typically have 2-3-nucleotide 3′ overhanging ends resembling the RNAse III processing products of long dsRNAs that normally initiate RNAi. When introduced into a cell, they assemble an endonuclease complex (RISC), which then guides target mRNA cleavage. As a consequence of degradation of the targeted mRNA, cells illustrating the specific phenotype associated with the suppression of the corresponding protein product are obtained. If the protein that is knocked down possesses an activity that attenuates cell growth, such knock down will result in increased growth of the cells, and vice versa.

To date, several distinct forms of RNA silencing have been found to regulate gene expression, to mediate antiviral responses, to organize chromosomal domains, and to restrain the spread of selfish genetic elements. For example, miRNAs derived from dsRNA precursors regulate gene expression in somatic cells by reducing translation and stability of protein-coding mRNAs.

MicroRNAs (miRNAs) are endogenously encoded RNAs that are about 22-nucleotide-long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Importantly, miRNAs are expressed in a highly tissue-specific or developmentally regulated manner, and this regulation is likely key to their predicted roles in eukaryotic development and differentiation.

The primary step in miRNA biogenesis is the nuclear cleavage of the “primary micro RNA” (pri-miRNA), liberating an approximately 70 nucleotide (nt) stem-loop intermediate known as “micro RNA precursor” (pre-miRNA). This processing step is performed by the RNase III endonuclease Drosha in conjunction with the dsRNA-binding protein “DiGeorge syndrome Critical Region gene 8” (DGCR8) in humans (Pasha in drosophila), leading to 5′ monophosphates and ˜2 nt 3′ overhangs characteristic for RNase III endonucleases.

The pre-miRNAs are then actively transported to the cytoplasm by Exportin-5 and the Ran-GTP cofactor. Subsequently, the mature miRNAs are excised by another RNase III endonuclease, Dicer, acting together with the dsRNA-binding protein tar-binding protein (TRBP) in humans or Loquacious (Logs) in flies. Depending on the species, the resulting short dsRNAs are about 21 to 28 nts in length.

For mRNA degradation, translational repression, or deadenylation, mature miRNAs or siRNAs are loaded into the RNA Induced Silencing Complex (RISC) by the RISC-loading complex (RLC). Subsequently, the guide strand leads the RISC to cognate target mRNAs in a sequence-specific manner and the Slicer component of RISC hydrolyses the phosphodiester bound coupling the target mRNA nucleotides paired to nucleotide 10 and 11 of the RNA guide strand. Slicer forms together with distinct classes of small RNAs the RNAi effector complex, which is the core of RISC. Therefore, the “guide strand” is that portion of the double-stranded RNA that associates with RISC, as opposed to the “passenger strand,” which is not associated with RISC. The target sequence contained in a reporter construct of the present invention is at least partially complementary to at least a portion of the guide strand.

Interfering RNA or small inhibitory RNA (RNAi) molecules (“RNAi molecules”) include short interfering RNAs (siRNAs), repeat-associated siRNAs (rasiRNAs), and micro-RNAs (miRNAs) in all stages of processing, including shRNAs, pri-miRNAs, and pre-miRNAs. These molecules have different origins: siRNAs are processed from double-stranded precursors (dsRNAs) with two distinct strands of base-paired RNA; siRNAs that are derived from repetitive sequences in the genome are called rasiRNAs; miRNAs are derived from a single transcript that forms base-paired hairpins. Base pairing of siRNAs and miRNAs may be perfect (i.e., completely complementary) or imperfect.

Depending on the application, any type of RNAi, including but not limited to siRNAs or shRNAs, can be used as RNAi triggers. The siRNAs have the advantage of being directly transfectable, chemically synthesized oligonucleotides that circumvent the need for cloning. siRNAs enter the miRNA processing pathway at a later stage, and bypass Drosha processing, Exportin-5 export, and, depending on their size, Dicer cleavage. However, when the objective is therapeutic, it is often preferable to use miRNA-based shRNAs as they tend to yield more effective silencing (Chang et al., Nature Methods, 2006, 3: 707-714). In addition, the small size of siRNAs and shRNAs, compared with traditional antisense molecules, prevents activation of the dsRNA-inducible interferon system present in mammalian cells. This helps avoid the nonspecific phenotypes normally produced by dsRNA larger than 30 base pairs in somatic cells. See, e.g., Elbashir et al., 2002, Methods Enzymol. 26: 199-213; McManus and Sharp, 2002, Nature Reviews 3: 737-747; Hannon, 2002, Nature 418: 244-251; Brummelkamp et al., 2002, Science 296: 550-553; Tuschl, 2002, Nature Biotechnology 20: 446-448; U.S. Publication No. 2002/0086356; WO 99/32619; WO 01/36646; and WO 01/68836.

As used herein, an “shRNA molecule” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, an shRNA forms a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

In some instances the precursor miRNA molecule may include more than one stem-loop structure. The multiple stem-loop structures may be linked to one another through a linker, such as, for example, a nucleic acid linker, a miRNA flanking sequence, other molecule, or some combination thereof.

Short hairpin RNAs can be designed to mimic endogenous miRNAs. Many miRNA intermediates can be used as models for shRNA or shRNAmir, including without limitation a miRNA comprising a backbone design of miR-15a, -16, -19b, -20, -23a, -27b, -29a, -30b, -30c, -104, -132s, -181, -191, -223 (see U.S. Publication No. 2005/0075492). The miR-30 natural configuration has proven especially beneficial in producing mature synthetic miRNAs. miR30-based shRNAs and shRNAmirs have complex folds, and, compared with simpler stem/loop style shRNAs, are more potent at inhibiting gene expression in transient assays. Moreover, they are associated with less toxic effects in cells.

In a preferred embodiment, shRNA molecules are designed based on the human miR-30 sequence, redesigned to allow expression of artificial shRNAs by substituting the stem sequences of the pri-miR-30 with unrelated base-paired sequences (Siolas et al., 2005, Nat. Biotech. 23: 227-231; Silva et al., 2005, Nat. Genet. 37: 1281-1288); Zeng et al. (2002), Molec. Cell 9: 1327-1333). The miR-30 architecture can be used to express miRNAs or siRNAs from RNA polymerase II promoter-based expression plasmids. See also Zeng et al., 2005, Methods Enzymol. 392: 371-380 (incorporated herein by reference).

The natural stem sequence of the miR-30 can be replaced with a stem sequence from about 16 to about 29 nucleotides in length, preferably from about 19 to 29 nucleotides in length. The loop sequence can be altered such that the length is from about 3 to about 23 nucleotides. In a preferred embodiment, the shRNA molecule is about 22 nucleotides in length. Thus, the invention may be practiced using short hairpin RNAs that are synthetically produced, as well as microRNA (miRNA) molecules that are found in nature and can be remodeled to function as synthetic silencing short hairpin RNAs.

“RNAi-expressing construct” or “RNAi construct” is a generic term that includes nucleic acid preparations designed to achieve an RNA interference effect. An RNAi-expressing construct comprises an RNAi molecule that can be cleaved in vivo to form an siRNA. Preferably, an RNAi construct is an expression vector capable of giving rise to an siRNA in vivo. Exemplary methods of making and delivering long or short RNAi constructs can be found, for example, in WO01/68836 and WO01/75164.

Other methods of RNA interference may also be used in the practice of this invention. See, e.g., Scherer and Rossi, Nature Biotechnology 21:1457-65 (2003) for a review on sequence-specific mRNA knockdown of using antisense oligonucleotides, ribozymes, DNAzymes, RNAi and siRNAs. See also, International Patent Application PCT/US2003/030901 (Publication No. WO 2004/029219 A2), filed Sep. 29, 2003 and entitled “Cell-based RNA Interference and Related Methods and Compositions”.

RNAi is also possible via gene expression cassettes expressing shRNA or shRNAmir. shRNA and shRNAmir are modeled on intermediate constructs of miRNA. Both are cleaved by DICER to form siRNAs and interact with the RISC complex in the same manner as siRNA.

When a nucleic acid construct encoding a short hairpin RNA is introduced into a cell, the cell incurs partial or complete loss of expression of the target gene. In this way, a short hairpin RNA functions as a sequence-specific expression inhibitor or modulator in transfected cells. The use of short hairpin RNAs facilitates the down-regulation of the target gene and allows for analysis of hypomorphic alleles. Short hairpin RNAs useful in the invention can be produced using a wide variety of well known RNAi techniques. The invention may be practiced using short hairpin RNAs that are synthetically produced as well as microRNA (miRNA) molecules that are found in nature and can be remodeled to function as synthetic silencing short hairpin RNAs. DNA vectors that express perfect complementary short hairpin RNAs (shRNAs or shRNAmirs) are commonly used to generate functional siRNAs.

In certain embodiments, useful interfering RNAs can be designed with a number of software programs, e.g., the OligoEngine siRNA design tool available at www.oligoengine.com. The siRNAs of this invention may be about, e.g., 19-29 base pairs in length for the double-stranded portion. In some embodiments, the siRNAs are shRNAs having a stem of about 19-29 base pairs and a nucleotide loop of about 4-34 bases. Preferred siRNAs are highly specific for a region of the target gene and may comprise a 19-29 base pair fragment of the mRNA of a target gene, with at least one, but preferably two or three, ase airbp mismatch with a nontarget gene-related sequence. In some embodiments, the preferred siRNAs do not bind to RNAs having more than three base pair mismatches with the target region.

In certain embodiments, artificial miRNA constructs based on miR-30 (microRNA 30) may be used to express precursor miRNA/shRNA. For example, Silva et al., 2005, Nature Genetics 37: 1281-88, have described extensive libraries of pri-miR-30-based retroviral expression vectors that can be used to down-regulate almost all known human (at least 28,000) and mouse (at least 25,000) genes (see RNAi Codex, a single database that curates publicly available RNAi resources, and provides the most complete access to this growing resource, allowing investigators to see not only released clones but also those that are soon to be released, available at http://codex.cshl.edu). Although such libraries are driven by RNA polymerase III promoters, they can be easily converted to the subject RNA polymerase II-driven promoters (see the Methods section in Dickins et al., 2005, Nature Genetics 37: 1289-95; also see page 1284 in Silva et al., 2005 supra).

In certain embodiments, the subject precursor miRNA cassette may be inserted within a gene encoded by the subject vector. For example, the subject precursor miRNA coding sequence may be inserted within an intron, the 5′- or 3′-UTR of a reporter gene, etc.

Other methods of RNAi may also be used in the practice of this invention. See, e.g., Scherer and Rossi, 2003, Nature Biotechnology 21: 1457-65 for a review on sequence-specific mRNA knockdown using antisense oligonucleotides, ribozymes, DNAzymes. See also, International Patent Application PCT/US2003/030901 (Publication No. WO 2004-029219 A2), filed Sep. 29, 2003 and entitled “Cell-based RNA Interference and Related Methods and Compositions.” See also Fewell et al., supra, for a description of inducible shRNA, in which the vector does not express the shRNA unless a specific reagent is added. Several studies investigating the function of essential genes using RNAi rely on inducible shRNA. For example, shRNAmir constructs can be created based on a tetracycline-responsive promotor system, such that shRNA expression is regulated by changing doxycycline levels.

In one embodiment of the present invention, a libary of RNAi molecules are introduced into a plurality of hepatocytes, using a vector known in the art. In certain embodiments, the vector is a viral vector. Exemplary viral vectors include adenoviral vectors, lentiviral vectors, or retroviral vectors. Many established viral vectors may be used to transfect foreign constructs into cells. The definition section below provides more details regarding the use of such vectors.

To facilitate the monitoring of the target gene knockdown, and the formation and progression of the cancer, cells harboring the RNAi-expressing construct may additionally comprise a marker construct, such as a fluorescent marker construct. The marker construct may express a marker, such as green fluorescent protein (GFP), enhanced green fluorescent protein (EGFP), Renilla Reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), citrine and red fluorescent protein from discosoma (dsRED). Other suitable detectable markers include chloramphenicol acetyltransferase (CAT), luciferase lacZ (13-galactosidase), and alkaline phosphatase. The marker gene may be separately introduced into the cell harboring the shRNA construct (e.g., co-transfected, etc.). Alternatively, the marker gene may be linked to the shRNA construct, and the marker gene expression may be controlled by a separate translation unit under an IRES (internal ribosomal entry site). In a preferred embodiment, the marker is a green fluorescent protein (GFP).

To facilitate the quantification of specific RNAi molecules in a complex population of cells infected with an entire library of RNAi molecules, each RNAi construct may additionally comprise a barcode. A barcode is a unique nucleotide sequence (generally 19-mer), linked to each shRNA. The barcode can be used to monitor the abundance of each shRNA via micoarray hybridization (Fewell et al., supra). In a preferred embodiment, each shRNA construct also comprises a unique barcode. For more information on the use of barcodes in shRNA pooled analyses, see Bernards et al., 2006, Nature Methods 3: 701-706, and Chang et al., 2006, Nature Methods 3: 707-714.

In certain embodiments, a plurality of hepatocytes are transformed (e.g.) transfected with a library of RNAi molecules, wherein each RNAi molecule targets a gene whose down-regulation is potentially associated with tumorigenesis, and the transformed cells are transplanted into an animal model. If an RNAi construct inhibits the expression of a tumor suppressor gene, the animal model receiving the transformed hepatocyte(s) may develop liver cancer (sometimes in conjunction with down-regulation of another tumor suppressor gene, or with the over-expression of an oncogene). Preferably, the liver cancer is recognizable by fluorescence or other detectable markers. From the tumors that arise, genomic DNA may be isolated, and the integrated hairpins may be amplified using polymerase chain reaction, cloned back into a vector, and then identified by sequencing.

Various methods may be used to determine the growth or viability of recipient cells expressing an RNAi-expressing construct in vitro. Such assays may be conducted using commercially available assay kits or methods well known to one or ordinary skill in the art. For example, cell viability can be determined by MTT assay or WST assay. The effect of the target gene knockdown can also be determined using cellular proliferation assays or cellular apoptosis/necrosis assays. In vitro cellular proliferation assays can be performed by determining the amount of cells in a culture over time. Cell numbers may be evaluated using standard techniques. Cellular apoptosis can be measured, for example, using a commercial apoptosis assay kit such as VYBRANT Apoptosis Assay Kit #3 (Molecular Probes). Cells can also be stained with P1 or DAP 1 to detect apoptotic nuclei.

In certain embodiments, recipient cells expressing an RNAi construct (e.g., a shRNA) against a target gene are sorted based on a selectable marker whose expression substantially matches the expression of the RNAi molecule. In one exemplary embodiment, the selectable marker is fluorescence—based. In one exemplary embodiment, the selectable marker is GFP. In one embodiment, cells harboring the selectable marker are sorted using fluorescence-activated cell sorting (FACS). FACS is a powerful system which not only quantifies the fluorescent signal but also separates the cells that contain preselected characteristics (such as fluorescence intensity, size and viability) from a mixed population. Laser light is directed at individual cells as they flow through the FACS. A light scatter pattern is generated when the dense nuclear material of the cell interferes with the path of the laser beam.

Recipient cells expressing an RNAi construct (e.g., a shRNA) against a target gene may be subsequently transplanted into a recipient non-human animal. Alternatively, after transfection, the cells may be injected subcutaneously into a recipient non-human animal. The size and growth of tumors in the recipient, the survival of tumor-free recipients, and overall survival of the recipient may then be observed to investigate the effect of target-gene-knockdown in vivo. The size and growth of tumors may be examined by any of many known methods in the art, such as histological methods, immunohistochemical methods, TUNEL-staining, etc. In certain embodiments, the non-human animal is a mouse. In certain embodiments, the recipient animal is an immuno-compromised animal, such as a nude mouse.

In certain aspects, the present invention provides methods for identifying tumor suppressor genes, libraries of RNAi molecules that can be used to identify tumor suppressor genes, and methods of making libraries of RNAi molecules that can be used to identify tumor suppressor genes. In other aspects, the present invention provides methods for identifying oncogenes, libraries of cDNA molecules that can be used to identify oncogenes, and methods of making libraries of cDNA molecules that can be used to oncogenes.

Such aspects integrate mouse models, cancer genomics, and in vivo RNAi and cDNA technologies for cancer gene discovery and validation. The methods and libraries were developed based on the premise that genomic deletions occurring in human tumors may be enriched for tumor suppressor genes and that genomic amplificatins occurring in human tumors may be enriched for oncogenes.

As described in Examples 2 and 3, focused shRNA libraries were produced by characterizing ˜100 human hepatocellular carcinomas (HCCs) for deletions using array-based screening. Genes contained in recurrent genomic deletions were then used to direct an in vivo RNAi screen for shRNAs capable of promoting tumorigenesis in a mosaic mouse model of HCC. As described in Examples 2 and 3, this approach proved to be highly effective, resulting in the functional validation of 14 new tumor suppressor genes, and the elucidation of pathways relevant to liver cancer and other tumor types. Furthermore all of the methods and compositions described herein as being useful for identification of tumor suppressor genes can also be used to identify oncogenes, by, for example, making or using libaraies of cDNA molecules as opposed to shRNA molecules, and by making or using cDNA libaries that contain sequences amplified in tumors as opposed to sequences that are deleted in tumors. One of skill in the art will be able to adapt all of the methods and compositions described herein, such as those provided in the Example, for use in identifying oncogenes.

The present invention also encompasses similar methods based on focused cDNA libaries produced by characterizing tumors for amplifications. Such methods are described in the Summary of the Invention and in the Claims.

Accordingly, in one aspect, the present invention provides a method for generating a library of RNAi molecules, such as an shRNA libray, that is useful for identifying tumor suppressor genes, the method comprising: identifying one or more genomic deletions, such as focal genomic deletions, in a population of cancer cells; cloning one or more RNAi molecules, such as shRNAs molecules, that target sequences located within the genomic deletions into an expression vector; and pooling the expression vectors to generate a library of RNAi molecules, such as an shRNA library, that is useful for identifying tumor suppressor genes.

Once a deletion has been identified, shRNAs targeting each gene within the deletion can be chosen/obtained from various sources. In one embodiment, the shRNAs are selected from existing libraries. For example, Silva et al. (2005, Nat. Genet. 37: 1281-1288), have described extensive libraries of pri-miR-30-based retroviral expression vectors that can be used to down-regulate almost all known human (at least 28,000) and mouse (at least 25,000) genes (see RNAi CODEX, a single database that curates publicly available RNAi resources, and provides the most complete access to this growing resource, allowing investigators to see not only released clones but also those that are soon to be released, available at the Cold Spring Harbor Laboratories website). Pools of shRNA useful to practice the method of the instant invention may be from the “the Cancer 1000” library, which was constructed by Steve Elledge and Greg Hannon. The “Cancer 1000” shRNA library includes a mixture of well characterized oncogenes and tumor suppressor genes in addition to many poorly-characterized genes somehow related to cancer, across many ontological groups, as compiled by literature mining. In another embodiment, the pools of shRNA useful to practice the method of the instant invention come from a cDNA library that includes a mixture of oncogenes. A similar library design rationale may be easily applied to construct RNAi libraries targeting genomes of other organisms. Examples of known tumor suppressors are p53, BRCA1, BRCA2, APC, p16^{INK4a}, PTEN, NF1, NF2, and RB1. These known tumor suppressors are expected to be identified and can serve as positive controls Negative controls can include shRNAs to genes not present in the organism's genome or empty vectors.

In another embodiment, the shRNAs may be designed de novo. The choice of the right primary sequence has an important role in determining the efficacy and specificity of the resulting RNAi response. Current features of design rules for RNAi molecules include the thermodynamic asymmetry of the RNA duplex, sequence homology of the seed sequence to its cognate target mRNA but not to other mRNAs, and a set of empirical single nucleotide position preferences. The thermodynamic asymmetry is important since only the strand with the less stable 5′ end is favorable or exclusively loaded into the RISC and will therefore serve as the guide strand. The seed sequence comprises nucleotide positions 2-8 of the guide strand and has been show to be the major specificity determinant of si- and shRNAs. Single nucleotide positional preferences include, for example, the A or U at position 10 of the guide strand that may promote catalytic RISC-mediated passenger strand and substrate cleavage.

In certain embodiments, useful interfering RNAs can be designed with a number of software programs, e.g., the OligoEngine siRNA design tool. Algorithms for in silenco prediction, or alorithms based on an empirically trained neural network, such as BIOPREDsi, can be used. Birmingham et al. (2007, Nat. Protocols 2: 2068-2078) provide a comprehensive overview of prediction algorithms. Effective RNAi molecules may also be designed using the Target Sensor assay described in PCT/US08/81193.

The shRNAs used may be about, e.g., 16-29 base pairs, preferably 19-29 base pairs in length for the double-stranded portion. In some embodiments, the siRNAs are shRNAs having a stem of about 19-29 base pairs and a nucleotide loop of about 3-34 bases. Preferred siRNAs are highly specific for a region of the target gene and may comprise a 19-29 base pair fragment of the mRNA of a target gene, with at least one, but preferably two or three, base pair mismatches with a nontarget gene-related sequence. In some embodiments, the preferred siRNAs do not bind to RNAs having more than three base pair mismatches with the target region.

Other methods of RNAi may also be used in the practice of this invention. See, e.g., Scherer and Rossi, 2003, Nature Biotechnology 21: 1457-65 for a review on sequence-specific mRNA knockdown using antisense oligonucleotides, ribozymes, DNAzymes. See also, International Patent Application PCT/US2003/030901 (Publication No. WO 2004-029219 A2) and Fewell et al., 2006, Drug Discovery Today 11: 975-982, for a description of inducible shRNA, in which the vector does not express the shRNA unless a specific reagent is added. Several studies investigating the function of essential genes using RNAi rely on inducible shRNA. For example, shRNAmir constructs can be created based on a tetracycline-responsive promotor system, such that shRNA expression is regulated by changing doxycycline levels.

In some embodiments, the RNAi molecules can be any RNAi molecules that can be used to inhibit expression of a target gene, for example siRNA molecules, shRNA molecules, shRNAmir molecules, and the like. In certain embodiments, such as those iluustrated in Examples 2 and 3, the RNAi molecules are shRNA molecules, such as a mir-30-based shRNA molecules.

In some embodiments the population of cancer cells screened for genomic deletions is a population of liver cancer cells, such as mouse or human hepatocellular carcinoma cells.

In some embodiments, the genomic deletions are identified using representational oligonucleotide microarray analysis (“ROMA”). In some embodiments, the genomic deletions are identified from genome database information or from other sources that describe genomic deletions associated with one or more cancers, such as mouse or human cancers. Any other means of identifiying cancer-associated genomic deletions may also be used.

Representational oligonucleotide microarray analysis (ROMA) is an ultra high resolution microarray-based comparative genomic hybridization (CGH) tool that evolved from a technique termed representational difference analysis (RDA) (Lucito et al., 2003, Genome Research 13:2291-2305; WO99/23256; U.S. Publications Nos. 2004/0137473, 2005/0196799, and 2005/0266444. Like RDA, ROMA is capable of detecting differences present in different genomes, for example, between cancer genomes present in different cancer cells in the same or different patients, or between cancer genomes and normal genomes. Thus, ROMA has applications in the detection of genetic variation, in or between individuals, caused by deletions or duplications/ amplifications of genomic DNA involving one or more genetic or genomic loci, which may be related to progression and prognosis of cancer or other inherited or somatic diseases. RDA compares two genomes by subtractive hybridization, and ROMA is a high-throughput method which employs microarray analysis and also compares two genomes by subtractive hybridization.

ROMA employs oligonucleotide probes that are representations of a genome, made by, for example, restriction enzyme cleavage of the genomic DNA, and the oligonucleotide probes can be designed in silico. An exemplary enzyme is BglII, of which the cleavage sites are relatively uniformly distributed in the human genome. Digestion of DNA with BglII can create about 200,000 representational fragments of the human genome, which are generally shorter than 1.2 kilobases, with an average spacing of about 17 kilobases. The representational oligonucleotide probes can be photoprinted onto microarray slides and then subjected to hybridization to genomic DNA of interest. Statistical data generated by the microarray hybridization can then be subjected to analysis by various algorithms, including, but not limited to, the circular binary segmentation that parses the probe ratio data into segments and creates a segmented genomic profile (Lucito et al., 2003).

In the present invention, ROMA can be used to identify deletions and/or DNA copy number alterations in tumor cells. Genes of interest include those embedded in recurrent focal deletions or in broader recurrent deletions. In a preferred embodiment, the tumor cells are human tumor cells. While human hapatocellular carcinomas are exemplified herein, the present methods can be extended to any tumor type. Therefore, tumor suppressors and/or cell cycle regulatory factors associated with a wide variety of cancers can be identified by the methods of the present invention.

In some embodiments, the expression vector used for shRNA expression is a viral vector and the method of transforming the mouse embryonic hepatocytes comprises infection. In other embodiments the expression vector may be a non-viral expression vector and the methods of transforming the mouse embryonic hepatocytes comprises transfection. shRNAs can be expressed from vectors to provide sustained silencing and high yield delivery into almost any cell type. In a preferred embodiment, the vector is a viral vector. Exemplary viral vectors include retroviral, including lentiviral, adenoviral, baculoviral and avian viral vectors. The use of viral vector-based RNAi delivery not only allows for stable single-copy genomic integrations but also avoids the non-sequence specific response via cell-surface toll-like receptor 3 (TLR3), which has raised many concerns for the specificity of siRNA mediated effects. In one embodiment of the present invention, a pool of shRNAs is introduced into embryonic hepatocytes using a vector known in the art. Other Examples of suitable vectors are described in the Examples section of this application.

Expression of endogenous miRNAs is controlled by RNA polymerase II (Pol II) promoters. It has been shown that shRNAs are also most efficiently driven by Pol II promoters, as compared to RNA polymerase III promoters (Dickins et al., 2005, Nat. Genet. 39: 914-921). Therefore, in some embodiments, the coding sequence of the shRNA molecules is controlled by an inducible promoter or a conditional expression system, including, without limitation, RNA polymerase type II promoters. Examples of useful promoters include tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. One embodiment uses tetracycline-responsive promoters, one of the most effective conditional gene expression systems in in vitro and in vivo studies.

In some embodiments, the shRNA expression vector, and/or the cells transformed with shRNA library molecules, may additionally comprise a marker or reporter construct, such as a fluorescent construct. The reporter construct may express a marker, such as green fluorescent protein (GFP), enhanced green fluorescent protein (EGFP), Renilla Reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), citrine and red fluorescent protein from discosoma (dsRED). Other suitable detectable markers include chloramphenicol acetyltransferase (CAT), luciferase lacZ (β-galactosidase), and alkaline phosphatase. The marker gene may be separately introduced into the cell harboring the shRNA construct (e.g., co-transfected, etc.). Alternatively, the marker gene may be on the shRNA construct, and the marker gene expression may be controlled by the same or a separate translation unit, for example, by an IRES (internal ribosomal entry site). In a preferred embodiment, the marker is a green fluorescent protein (GFP).

To facilitate the quantification of specific shRNAs in a complex population of cells infected with a library of shRNAs, each shRNA construct may additionally comprise a barcode. A barcode is a unique nucleotide sequence (generally a 19-mer), linked to each shRNA. The barcode can be used to monitor the abundance of each shRNA via micoarray hybridization (Fewell et al., 2006, Drug Discovery Today 11: 975-982). In a preferred embodiment, each shRNA construct also comprises a unique barcode. For more information on the use of barcodes in shRNA pooled analyses, see Bernards et al., 2006, Nature Methods 3: 701-706, and Chang et al., 2006, Nature Methods 3: 707-714.

In some embodiments, the RNAi library, such as the shRNA library, comprises up to about 100 different RNAi sequences, such as shRNA sequences. In some embodiments, the RNAi library, such as the shRNA library, comprises up to about 90 different RNAi sequences, such as shRNA sequences. In some embodiments, the library of RNAi molecules, such as the shRNA library, comprises up to about 80 different RNAi molecule sequences, such as shRNA sequences. In some embodiments, the library comprises up to about 70 different RNAi sequences, such as shRNA sequences. In some embodiments, the RNAi library, such as the shRNA library, comprises up to about 60 different RNAi sequences, such as shRNA sequences. In some embodiments, the RNAi library, such as the shRNA library, comprises up to about 55 different RNAi sequences, such as shRNA sequences. In some embodiments, the RNAi library, such as the shRNA library, comprises up to about 50 different RNAi sequences, such as shRNA sequences. In some embodiments, the RNAi library, such as the shRNA library, comprises up to about 45 different RNAi sequences, such as shRNA sequences. In some embodiments, the RNAi library, such as the shRNA library, comprises up to about 40 different RNAi sequences, such as shRNA sequences. In some embodiments, the RNAi library, such as the shRNA library, comprises up to about 35 different RNAi sequences, such as shRNA sequences. In some embodiments, the RNAi library, such as the shRNA library, comprises up to about 30 different RNAi sequences, such as shRNA sequences. In some embodiments, the RNAi library, such as the shRNA library, comprises about 48 different RNAi sequences, such as shRNA sequences.

In some aspects, the present invention provides libraries of RNAi molecules, such as shRNA libraries, generated using the methods described above. For example, in one aspect, the present invention provides an shRNA library that comprises, or consists essentially of, or consists of shRNA molecules that target mRNAs transcribed from one or more genes located within one or more cancer-associated focal genomic deletions, such as focal genomic deletions associated with liver cancer or breast cancer. In one aspect, the shRNA molecules in the library target mRNAs transcribed from one or more genes selected from the 362 genes listed in Table 3. In another aspect, the the shRNA molecules in the library target mRNAs transcribed from one or more genes located within one or more of the 58 cancer-associated focal genomic deletions listed in Table 3.

The present invention also provides methods for identifying tumor suppressor genes using the RNAi libraries described above. For example, the present invention provides a method of identifying a tumor suppressor gene, comprising: identifying one or more genomic deletions, such as focal genomic deletions, in a population of cancer cells; cloning one or more RNAi molecules, such as shRNAs, that target sequences located within the one or more genomic deletions into an expression vector; pooling the expression vectors to generate an RNAi library; transforming the RNAi library into mouse embryonic hepatocytes; introducing transformed mouse embryonic hepatocytes into mice; amplifying one or more RNAi molecules from a tumor generated in a transformed mouse; and identifying a gene sequence to which the shRNA is complementary; whereby the gene identified can be a tumor suppressor gene.

In some embodiments, the mouse embryonic hepatocytes into which the RNAi library are transformed as are p53-negaative and/or Myc positive, such as those in the mosaic mouse model of hepatocellular carcinoma described in Zender et al. 2005 (Cold. Spring. Harbor. Symp. Quant. Biol. 70; p 251-261) and Zender et al. 2006 (Cell. 125; p 1253-1267) the contents of which are hereby incorporated by reference.

In one embodiment, mouse embryonic hepatocytes are transformed with a pool of shRNAs targeting candidate turmor suppressor genes, and the transformed cells are reconstituted into mice. Mice receiving cells transformed with tumor suppressor gene-targeting shRNAs, and that optionally also overexpress Myc and/or are p53−/−, develop tumors. These may be recognizable by green fluorescence where GFP markers are used. From the tumors that arise, genomic DNA can be isolated, and the integrated hairpins can be amplified, for example using polymerase chain reaction, cloned back into a vector, and then identified by sequencing.

Various methods may be used to determine the growth or viability of recipient cells expressing an RNAi-expressing construct in vitro. Such assays may be conducted using commercially available assay kits or methods well known to one or ordinary skill in the art. For example, cell viability can be determined by MTT assay or WST assay. The effect of the target gene knockdown can also be determined using cellular proliferation assays or cellular apoptosis/necrosis assays. In vitro cellular proliferation assays can be performed by determining the amount of cells in a culture over time. Cell numbers may be evaluated using standard techniques. Cellular apoptosis can be measured, for example, using a commercial apoptosis assay kit such as VYBRANT Apoptosis Assay Kit #3 (Molecular Probes). Cells can also be stained with P1 or DAP1 to detect apoptotic nuclei.

In certain embodiments, recipient cells expressing an RNAi construct (e.g., a shRNA) against a target gene are sorted based on a selectable marker whose expression substantially matches the expression of the RNAi molecule. In one exemplary embodiment, the selectable marker is fluorescence-based. In one exemplary embodiment, the selectable marker is GFP. In one embodiment, cells harboring the selectable marker are sorted using fluorescence-activated cell sorting (FACS). FACS is a powerful system which not only quantifies the fluorescent signal but also separates the cells that contain preselected characteristics (such as fluorescence intensity, size and viability) from a mixed population. Laser light is directed at individual cells as they flow through the FACS. A light scatter pattern is generated when the dense nuclear material of the cell interferes with the path of the laser beam.

Recipient cells expressing an RNAi construct (e.g., a shRNA) against a target gene may be subsequently transplanted into a recipient non-human animal. Alternatively, after shRNA transformatioj, the cells may be injected subcutaneously into a recipient non-human animal. The size and growth of tumors in the recipient, the surival of tumor-free recipients, and overall survival of the recipient may then be observed to investigate the effect of target-gene-knockdown in vivo. The size and growth of tumors may be examined by any of many known methods in the art, such as histological methods, immunohistochemical methods, TUNEL-staining, etc. In certain embodiments, the non-human animal is a mouse. In certain embodiments, the recipient animal is an immuno-compromised animal, such as a nude mouse.

Identified RNAi molecules and tumor suppressor genes may be validated by introduction into cells and assessment for knockdown, which may be done by immunoblotting or QPCR. If positive, the individual RNAi molecule may be further evaluated for their activities in animal models.

The candidate genes may be further assessed in vitro or in vivo to ascertain the mechanism by which knockdown of these putative genes is tumorigenic. Such processes will elucidate whether the tumorigenesis is due to apoptotic defects or proliferation advantage. For example, response to growth factor withdrawal, DNA damage response to cytotoxic drugs, or activity of downstream targets would be further examined. In addition, deletions or mutations in human tumors can be explored and compared, using, for example, the ROMA database and human tumor samples.

In certain aspects, the present invention is directed to tumor suppressor genes identified using some of the methods described herein, as outlined in Examples 2 and 3. Accordingly, in certain aspects, the present invention provides a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9. The present invention also provides other tumor suppressor genes that were identified as being enriched to greater than 5% in tumors using the methods of the invention, as listed in Tables 1 and 4 and other genes located in cancer associated deletions as described in the Examples section of this application, including in Table 1-5 and 7. The sequences of each of these tumor suppressor genes are known in the art and may be obtained from GenBank and other databases of gene, nucleotide, and protein sequences. The table below provides some such sequences. The contents of each of the database entries listed in the table below are hereby incorporated by reference in their entireties. Additionally, one of skill in the art can readily identify other mRNA, cDNA, gene, or protein sequences for each of the listed tumor suppressor genes using GenBank and other publicly available databases.

| Tumor | | |
|---|---|---|
| Suppressor | mRNA or cDNA Sequence | Protein Sequence |
| Gene | (Accession No.) | (Accession No.) |
| XPO4 | BC113573.1 (HUMAN) | NP071904.4 (HUMAN) |
| | NM020506.1 (MOUSE) | NP065252.1 (MOUSE) |
| FGF6 | NM010204.1 (MOUSE) | NP034334.1 MOUSE |
| | NM020996.1 (HUMAN) | NP066276.2 HUMAN |
| WDR49 | NM178824.3 (HUMAN) | NP 849146.1 HUMAN |
| | XM143339.5 (MOUSE) | XP143339.5 MOUSE |
| ARMCX2 | NM 177949.2 (HUMAN) | AAH1254.1 HUMAN |
| | BC054839.1 (MOUSE) | CAM20586.1 MOUSE |
| FSTL5 | BC036502.2 (HUMAN) | AAH36502.1 HUMAN |
| | NM 178673.3 (MOUSE) | AAH75640.1 MOUSE |
| NRSN2 | NM024598.2 (HUMAN) | AAH01963.1 HUMAN |
| | BC088982.1 (MOUSE) | EDL05964.1 MOUSE |
| WDR37 | NM014023.3 (HUMAN) | NP054742.2 HUMAN |
| | NM001039388.1 (MOUSE) | AAH46236.1 MOUSE |
| ARMCX1 | NM016608.1 (HUMAN) | NP 057692.1 HUMAN |
| | BC068228.1 (MOUSE) | NP 00159850.1 MOUSE |
| GJD4 | BC035898.1 (HUMAN) | NP 699199.2 HUMAN |
| | NM153086.5 (MOUSE) | AAI 37985.1 MOUSE |
| ZBBX | NM024687.2 (HUMAN) | AAI44445.1 HUMAN |
| | NM 172525.2 (MOUSE) | AAH50789.1 MOUSE |
| GLO1 | NM 006708.2 (HUMAN) | NP006699.2 HUMAN |
| | BC024663.1 (MOUSE) | NP 001107032.1 MOUSE |
| DDX20 | NM 007204.4 (HUMAN) | NP 009135.4 HUMAN |
| | NM017397.3 (MOUSE) | NP 059093.3 MOUSE |
| SET | AB015613.1 (MOUSE) | CAG46847.1 (HUMAN) |
| | CR542050.1 (HUMAN) | BAA34736.1 (MOUSE) |
| BTBD9 | BC057897.1 (MOUSE) | AAI01358.1 (HUMAN) |
| | BC101354.2 (HUMAN) | NP_766206.1 (MOUSE) |

In another embodiment, the the present invention provides proteins or peptides encoded by the listed tumor suppressor genes (hereinafter referred to as “tumor suppressor polypeptides” or “tumor suppressor proteins”), and analogs of these tumor suppressor polypeptides. The tumor suppressor protein analogs may be based on any of the tumor suppressor polypeptides encoded by the genes listed above, or sequences that have about 70% or more, or about 75% or more, or about 80% or more, or about 85% or more, or about 90% or more, or about 91%, 92%, 93%, 94%, 95%, 96%, 97%, 98%, or 99% or more amino acid identity with any of the tumor suppressor polypeptides listed above. In addition, the tumor suppressor polypeptide analogs may be based on tumor suppressor polypeptides that have been altered in some useful way, such as by including a fusion protein, by adding, removing, or substituting certain amino acids (for example to increase stability of activity), by conjugating the tumor suppressor polypeptide with other substances, e.g. polyethylene glycol, and the like. All such variant forms (including homologues, orthologs, fragments, mutants, and the like) of tumor suppressor polypeptides that retain anti-proliferative and/or anti-tumor activity are within the scope of the present invention. The ability of such variant forms to inhibit cellular proliferation and/or tumor growth can readily be assessed using the methods descrtibed in the specification and in Examples 2 and 3, for example using the methods described in Examples 2 and 3 for validationof the tumor suppressor activity of XPO4.

In another aspect, the invention also provides the E1F5A2 oncogene, which, as demonstrated herein, is over-expressed and/or amplified in liver cancer and breast cancer, and which is a target of the XPO4 oncogene. The human E1F5A2 oncogene is described in, Jenkins et al., “Human eIF5A2 on chromosome 3q25-q27 is a phylogenetically conserved vertebrate variant of eukaryotic translation initiation factor 5A with tissue-specific expression” Genomics. (2001) 71(1):p 101-9, and Clement et al. “Identification and characterization of eukaryotic initiation factor 5A-2” Eur J Biochem. (2003) 270(21): p 4254-63, the contents of which are hereby incorporated by reference. The sequences of the E1F5A2 oncogene is known in the art and may be obtained from GenBank and other databases of gene, nucleotide, and protein sequences.

In another embodiment, the the present invention provides proteins or peptides encoded by the E1F5A2 oncogene (hereinafter referred to as “E1F5A2 oncogene product” or “E1F5A2 oncogenic protein” or the “E1F5A2 protein”), and analogs of thereof. The E1F5A2 protein analogs may be based on any of the E1F5A2 protein encoded by the E1F5A2 genes listed above, or sequences that have about 70% or more, or about 75% or more, or about 80% or more, or about 85% or more, or about 90% or more, or about 91%, 92%, 93%, 94%, 95%, 96%, 97%, 98%, or 99% or more amino acid identity with any of the such sequences. In addition, the tumor E1F5A2 protein analogs may be based on E1F5A2 proteins that have been altered in some useful way, such as by including a fusion protein, by adding, removing, or substituting certain amino acids (for example to increase stability of activity), by conjugating the tumor suppressor polypeptide with other substances, e.g. polyethylene glycol, and the like. All such variant forms (including homologues, orthologs, fragments, mutants, and the like) of E1F5A2 proteins that retain ocogenic and/or pro-proliferative and/or pro-tumor activity are within the scope of the present invention. The ability of such variant forms to inhibit cellular proliferation and/or tumor growth can readily be assessed using standard methods known in the art for studying the effects of oncogenes or oncogenic proteins, including, for example, the animal models described herein.

In certain aspects, the present invention provides methods for inhibiting cell proliferation. For example, the present invention provides methods for inhibiting cell proliferation comprising upregulating expression of a gene selected from the group consisting of XPO4, FGF6, WDR49, ARMCX2, FSTL5, NRSN2, WDR37, ARMCX1, GJD4, ZBBX, GLO1, DDX20, SET, and BTBD9 in a cell. In some embodiments, the gene is XPO4. In some embodiments, the step of upregulating XPO4 expression comprises introducing into the cell a nucleic acid molecule encoding a physiologically active XPO4 polypeptide. In some embodiments, the cell is a cancer cell, such as a liver cancer cell or a breast cancer cell. The cancer cell my be in vitro or may in a subject, such as a mouse or human subject.

In some embodiments, the present invention provides a method of inhibiting cell proliferation comprising administering to a cell a a physiologically active polypeptide encoded by a gene selected from the group consisting of XPO4, FGF6, WDR49, ARMCX2, FSTL5, NRSN2, WDR37, ARMCX1, GJD4, ZBBX, GLO1, DDX20, SET, and BTBD9, or an analog thereof, thereby inhibiting cell proliferation. In some embodiments, the method comprises administering a physiologically active XPO4 polypeptide, or an analog thereof, to the cell. In some embodiments, the cell is a cancer cell, such as a liver cancer cell or a breast cancer cell. The cancer cell my be in vitro or may in a subject, such as a mouse or human subject.

Another aspect of the invention is a method of treating cancer comprising the steps of determining the status in cancerous tissue of one or more of the tumor suppressor genes described herein or identified by the screening method described herein, and if the expression or activity of any of the tumor suppressors is reduced in cancerous tissue in comparison to a control (e.g., a normal tissue), increasing the expression or activity of said tumor suppressor(s).

In one embodiment, the expression of a tumor suppressor is increased by introducing the tumor suppressor into the cancerous tissue. In a particular embodiment, the tumor suppressor protein or a physiologically active fragment, analog, or mutant thereof is administered. In another particular embodiment, the tumor suppressor gene or a fragment or mutant thereof that encodes a physiologically active polypeptide is introduced into the cancer tissue and expressed. In yet another embodiment, known upstream factors of an identified tumor suppressor is modulated to increase the tumor suppressor expression.

More specifically, an embodiment of the invention is a method for treating cancer comprising the steps of determining the status in cancerous tissue of one or more of the tumor suppressors described in Table 1 of Example 2. In one exemplary embodiment, the tumor suppressor is exportin 4 (Xpo4).

Another aspect of the invention is a method of treating cancer comprising: determining in cancerous tissue the expression of one or more tumor suppressor genes described herein or identified by the screening method described herein, the expression of which gene or genes are increased or decreased in comparison to a control (e.g., a normal tissue), and administering a therapeutic agent that is known to be effective in treating such cancers that are associated with the increased or decreased expression of such gene or genes. Alternatively, an aspect of the invention is a method of treating cancer comprising: determining in cancerous tissue the expression of one or more tumor suppressor genes described herein or identified by the screening method described herein, the expression of which gene or genes are decreased in comparison to a control (e.g., a normal tissue), and administering a therapeutic agent that is known not to antagonize the gene or genes identified herein.

More particularly, an embodiment of this aspect of the invention can be practiced using the tumor suppressor genes listed in the Table 1 of Example 2, or any other genes that are identified using the screening method described herein. More particularly, said tumor suppressor gene is Xpo4.

The expression level of a tumor suppressor gene be measured by mRNA level, protein level, activity level, or other quantity reflected in or derivable from the gene or protein expression data. For example, the mRNA level of a tumor suppressor gene may be measured using microarray technology that is well known in the art. Briefly, in a typical microarray experiment, a microarray is hybridized with differentially labeled RNA or DNA populations derived from two different samples. Most commonly, RNA (either total RNA or mRNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used. cDNA derived from one sample is labeled with one fluor while cDNA derived from a second sample is labeled with the second fluor. Similar amounts of labeled material from the two samples are cohybridized to the microarray. In the case of a microarray experiment in which the samples are labeled with Cy5 (which fluoresces red) and Cy3 (which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray.

Alternatively, the mRNA level a tumor suppressor gene can be measured by polymerase chain reaction (PCR), a technique well known in the art. Briefly, one or more sets of oligonucleotide primers are annealed to a target sequence of interest, and the annealed primers are extended simultaneously to generate double-stranded (ds) copies of the target sequence. The primers are extended by a thermal-stable polymerase (McPherson, M. Ed. (1995) PCR 2: A Practical Approach, IRL Press at Oxford University Press, Oxford). The primers may be about 5-50 nucleotides in length. Real-time polymerase chain reaction, also called quantitative real time PCR (QRT-PCR) or kinetic polymerase chain reaction, may be highly useful to determine the expression level of a target gene because the technique can simultaneously quantify and amplify a specific part of a given polynucleotide. The QRT-PCR procedure follows the general pattern of polymerase chain reaction, but the DNA is quantified after each round of amplification. Two common methods of quantification are the use of fluorescent dyes that intercalate with double-strand DNA, and modified DNA oligonucleotide probes that fluoresce when hybridized with a complementary DNA. QRT-PCR can be combined with reverse transcription polymerase chain reaction to quantify low abundance messenger RNA (mRNA), enabling one to quantify relative gene expression at a particular time, or in a particular cell or tissue type.

The expression level of tumor suppressor may also be measured by protein level using any art-known method. Traditional methodologies for protein quantification include 2-D gel electrophoresis, mass spectrometry and antibody binding. Preferred methods for assaying target protein levels in a biological sample include antibody-based techniques, such as immunoblotting (western blotting), immunohistological assay, enzyme linked immunosorbent assay (ELISA), radioimmunoassay (RIA), or protein chips. Gel electrophoresis, immunoprecipitation and mass spectrometry may be carried out using standard techniques, for example, such as those described in Molecular Cloning A Laboratory Manual, 2^{nd} Ed., ed. by Sambrook, Fritsch and Maniatis (Cold Spring Harbor Laboratory Press: 1989), Harlow and Lane, Antibodies: A Laboratory Manual (1988 Cold Spring Harbor Laboratory), G. Suizdak, Mass Spectrometry for Biotechnology (Academic Press 1996), as well as other references cited herein.

The expression level of a tumor suppressor gene can also be measured by the activity level of the gene product using any art-known method.

In certain embodiments, it may be useful to compare the expression level of a tumor suppressor gene to a control. The control may be a measure of the expression of the tumor suppressor gene in a quantitative form (e.g., a number, ratio, percentage, graph, etc.) or a qualitative form (e.g., band intensity on a gel or blot, etc.). A variety of controls may be used. Levels of a tumor suppressor gene expression from a healthy individual may also be used as a control. Alternatively, the control may be expression levels of tumor suppressor gene from the individual being treated at a time prior to treatment or at a time period earlier during the course of treatment. Still other controls may include expression levels present in a database (e.g., a table, electronic database, spreadsheet, etc.).

In another aspects, the invention provides methods for identifying a subject who is suffering from, or at risk of developing cancer, such as liver cancer or breast cancer.

For example, in one embodiment, the present invention provides a method for determining whether a subject is suffering from, or at risk of developing, liver or breast cancer, the method comprising obtaining genomic DNA from the subject and determining whether the genomic DNA contains a deletion of one or more of genes selected from the group consisting of XPO4, FGF6, WDR49, ARMCX2, FSTL5, NRSN2, WDR37, ARMCX1, GJD4, ZBBX, GLO1, DDX20, SET, and BTBD9, whereby a deletion of one or more of the genes indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

In other embodiments, the present invention provides a method for determining whether a subject is suffering from, or at risk of developing, liver or breast cancer, the method comprising obtaining a sample comprising mRNA from the subject, and measuring the level of an mRNA selected from the group consisting of XPO4, FGF6, WDR49, ARMCX2, FSTL5, NRSN2, WDR37, ARMCX1, GJD4, ZBBX, GLO1, DDX20, SET, and BTBD9 mRNAs in the sample, whereby a level of expression of one or more of the mRNAs that is lower than the level of expression of the mRNA in a control subject, indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

In other embodiments, the present invention provides a method for determining whether a subject is suffering from, or at risk of developing, liver or breast cancer, the method comprising obtaining a sample from the subject and measuring the level of a protein selected from the group consisting of the proteins encoded by the XPO4, FGF6, WDR49, ARMCX2, FSTL5, NRSN2, WDR37, ARMCX1, GJD4, ZBBX, GLO1, DDX20, SET, and BTBD9 genes in the sample, whereby a level of the protein that is lower than the level of the same protein in a control subject, indicates that the subject is suffering from, or at risk of developing, liver or breast cancer. Such a method can also be used in accordance with the invention to predict a cancer patients' survival. For example, as illustrated in the Table 6, XPO4 deletion is associated with poor survival outcomes in breast cancer patients.

Yet another aspect of the invention is a pharmaceutical composition comprising a therapeutic agent for the treatment of cancer, which composition has specific utility to treat such cancer that has certain status regarding one or more tumor suppressors identified using the method described herein.

One embodiment of the invention is a pharmaceutical composition for the treatment of cancer in which the expression of said tumor suppressor is less than a control (e.g., in normal tissue), comprising a tumor suppressor protein or a physiologically active fragment, analog, or mutant thereof. Another particular embodiment is a pharmaceutical composition for the treatment of cancer in which the expression or activity of a tumor suppressor is less than a control (e.g., in normal tissue), comprising a tumor suppressor gene or a fragment or mutant thereof that encodes a physiologically active polypeptide is introduced into the cancer tissue and expressed. In yet another embodiment, a pharmaceutical composition comprises one or more therapeutic agents that modulate known upstream factors of an identified tumor suppressor to increase the tumor suppressor expression.

In another aspect, the invention provides compositions, such as pharmaceutical compositions, that can be used to inhibit proliferation of a cell, such a cancer cell, for example liver cancer cells or breast cancer cells. Such compositions can comprise an agent that upregulates expression of a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9, or can comprise an nucleic acid sequence that encodes a tumor suppressor gene selected from the group consisting of XPO4, FGF6, WDR49, ARMCX2, FSTL5, NRSN2, WDR37, ARMCX1, GJD4, ZBBX, GLO1, DDX20, SET, and BTBD9 or an analog thereof, or can comprise a polypeptide encoded by a tumor suppressor gene selected from the group consisting of XPO4, FGF6, WDR49, ARMCX2, FSTL5, N. RSN2, WDR37, ARMCX1, GJD4, ZBBX, GLO1, DDX20, SET, and BTBD9 or an analog thereof. In another embodiment, such compositions can comprise an agent that inhibits the expression and/or activity of the EIF5A2 gene or the EIF5A2 gene product, including, but not limited to, interfering RNA molecules that target EIF5A2, such as an shRNA or an siRNA molecules. In other aspects, the present invention provides compositons that comprise combinations of one or more of the above, and also provides compositions that comprise one or more of the above in combination with other agents useful for inhibiting cell proliferation and/or for treating cancer, including, but not limited to, chemotherapeutic agents and the like.

More particularly, an embodiment of this aspect of the invention can be practiced using the tumor suppressor genes listed in the Table 1 of Example 2, or any other genes that are identified using the screening method described herein. More particularly, said tumor suppressor gene is XPO4.

The invention is also described by the following examples, which are included merely for purposes of illustration, and which are not intended to limit the invention. Those skilled in the art will recognize, or be able to ascertain using no more than routine experimentation, many equivalents to the specific embodiments of the invention described herein, including in the below Examples and in the claims. Such equivalents are intended to be within the scope of the present invention.

### Examples

### Example 1

### Generation and Transplantation of Genetically Altered Liver Progenitor Cells

To determine whether genetically modified hepatoblasts could colonize recipient livers, a protocol was used that optimizes engraftment of transplanted cells in the recipient liver. Embryonic hepatoblasts express high E-Cadherin levels on their cell surface, which enables these cells to be isolated to high purity from fetal livers using magnetic bead selection. (Nitou et al. “Purification of fetal mouse hepatoblasts by magnetic beads coated with monoclonal anti-e-cadherin antibodies and their in vitro culture.” Exp. Cell Res. 279, 330-343. (2002)). These cells express markers characteristic of bi-potential oval cells, the presumed cellular target of transformation in the adult rodent liver.

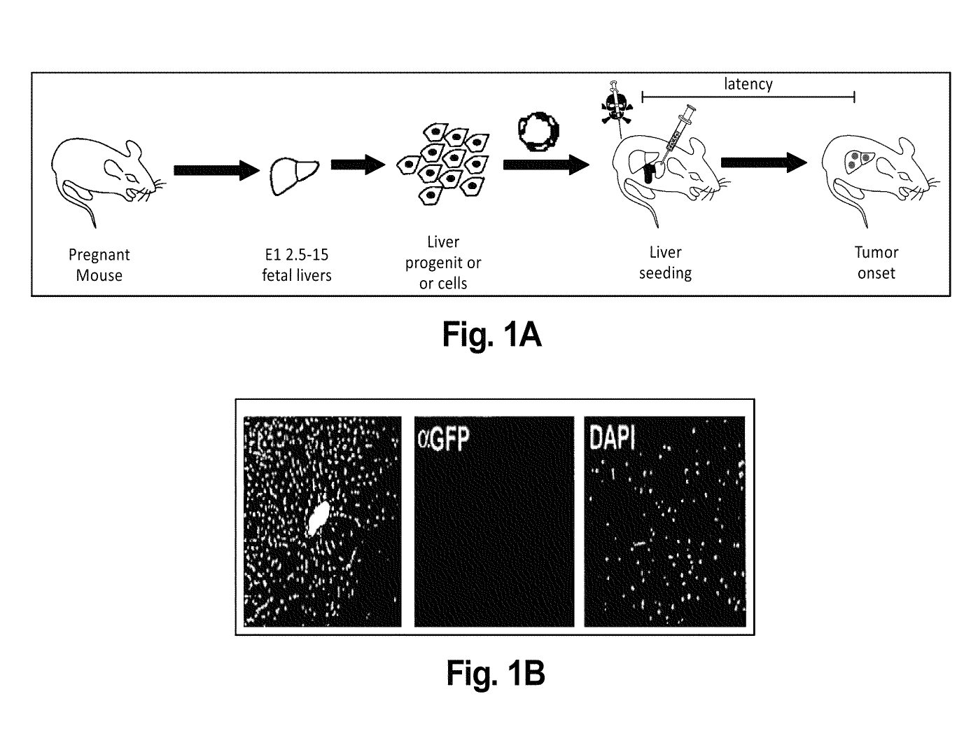

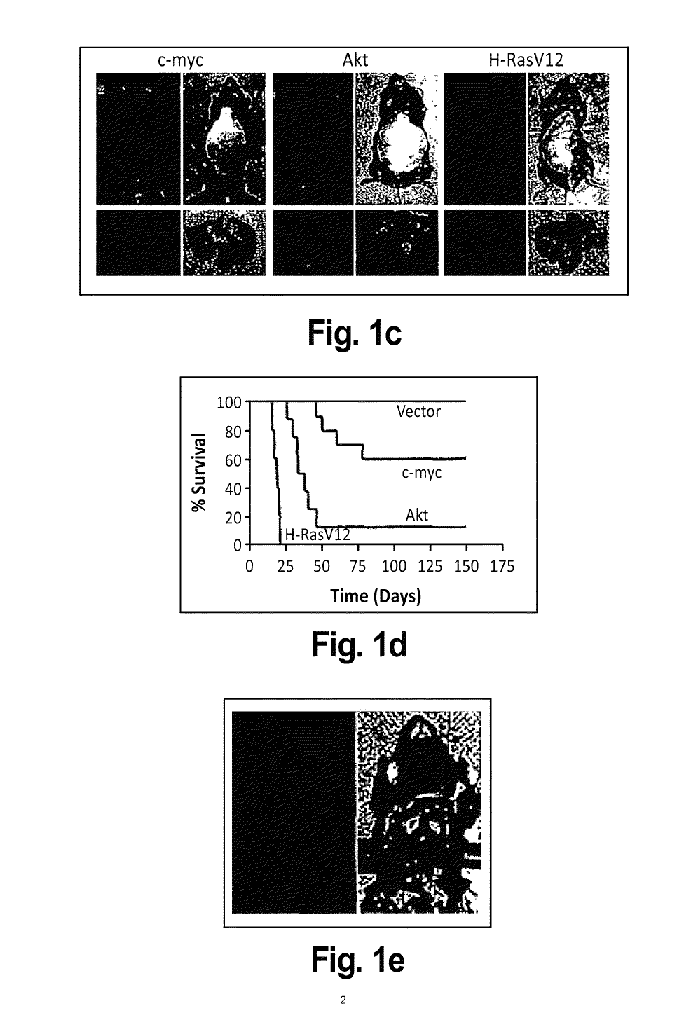

Animals were pretreated with retrorsine, an alkaloid that exerts a strong and persistent block of native hepatocyte proliferation and increases the competitive advantage of transplanted cells. Ten days after the last retrorsine treatment, 2×10⁶ GFP-tagged E-Cadherin⁺ liver progenitor cells were delivered to the liver by intrasplenic injection. Using this protocol, one week after injection approximately one percent of the host liver consisted of “seeded” GFP-positive cells that were embedded within the normal liver architecture (FIG. 1C).

Generation of Liver Carcinomas from Transplanted Liver Progenitor Cells

Hepatoblasts were isolated from p53−/−fetal livers and the cells were transduced with retroviruses co-expressing Myc (c-myc), activated Akt (Akt1), or oncogenic Ras (H-rasV12) (each of which affect signaling pathways altered in human liver cancer) and a GFP reporter. As above, these transduced cell populations were transplanted into retrorsine treated mice (see FIG. 1A). To further facilitate expansion of the transplanted cells, recipient mice were treated with CCI4 (Guo et al. “Liver repopulation after cell transplantation in mice treated with retrorsine and carbon tetrachloride.” Transplantation 73, 1818-1824.(2002)) and monitored for signs of disease by abdominal palpation of the liver and whole body fluorescence imaging. Although p53−/− hepatoblasts were not tumorigenic in our system, each of the cell populations that also expressed an oncogene eventually produced GFP-positive tumors in the livers of recipient mice (FIG. 1C, top).

Gross pathological analysis of explanted livers revealed that Myc-expressing tumors differ significantly from those expressing Akt or Ras (FIG. 1C, bottom). First, Myc-expressing tumors grow primarily as unilocular tumors, whereas Akt- and Ras-derived tumors show aggressive, multilocular and infiltrative intrahepatic growth. Second, the innate tumorigenicity of p53−/− liver progenitor cells expressing Myc was significantly lower than those expressing Akt or Ras; thus, Akt or Ras trigger the development of liver carcinomas with an efficiency of nearly 100%, while Myc produced tumors at a penetrance around 40% (FIG. 1D). In most instances, GFP-positive cells derived from these tumors could be readily grown in culture, and subsequently formed secondary tumors upon sub-cutaneous injection into immunocompromised mice or direct intrahepatic injection into syngeneic recipients (data not shown).

### Murine Liver Carcinomas Histopathologically Resemble Features of Human HCC

To determine whether the murine tumors produced from liver progenitors resemble human liver cancer, a panel of hematoxylin/eosin (H&E) stained sections derived from primary Myc-induced murine hepatomas were examined by an experienced liver pathologist. These tumors were classified as moderately well to poorly differentiated HCCs with a mostly solid, sometimes mixed solid/trabecular growth pattern. A smaller proportion of tumors revealed growth patterns resembling trabecular or pseudoglandular HCC (data not shown). All tumors examined stained positive for cytokeratin 8, confirming they were derived from the liver lineage. Furthermore, transplanted tumors retained their HCC histology when injected orthotopically into the liver, or subcutaneously into immunocompromised mice (data not shown). These findings confirm that ex vivo manipulated liver progenitor cells can produce tumors that recapitulate the histopathology of human HCC.

### ROMA Identifies Spontaneous Mutations in a Subset of Murine Liver Carcinomas

Epithelial cancers require a series of genetic alterations during clonal evolution to an advanced disease. To molecularly characterize the murine HCCs described above, spontaneously acquired lesions in those cancers were analyzed using representational oligonucleotide microarray analysis (ROMA), a genome-wide scanning method capable of identifying copy number alterations in tumor cells at high resolution (Lucito et al. “Representational oligonucleotide microarray analysis: a high-resolution method to detect genome copy number variation.” Genome Res. 13, 2291-2305.(2003)). Each human or mouse ROMA array consisted of 85K oligonucleotide probes designed to the UCSC April/2003 draft assembly of human genome and the UCSC February/2003 mouse genome, respectively, allowing genome scanning at a theoretical resolution of ˜35 kb.

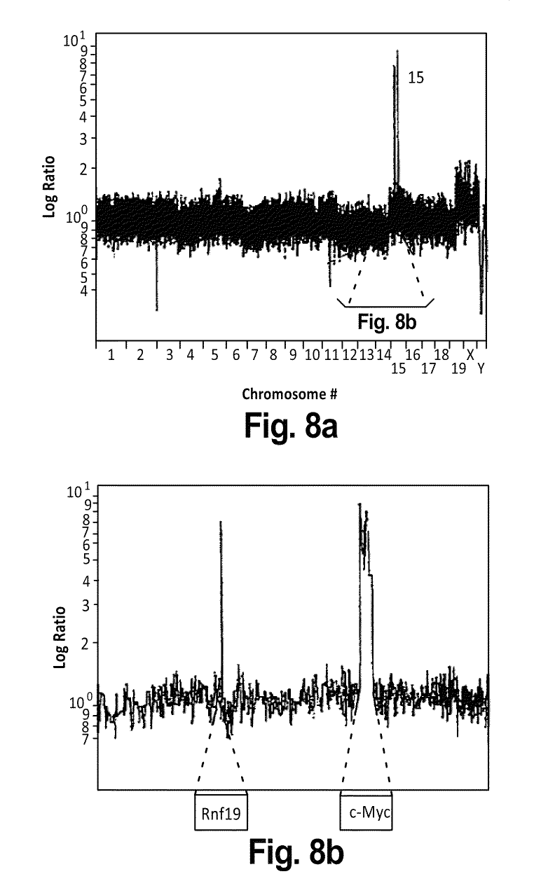

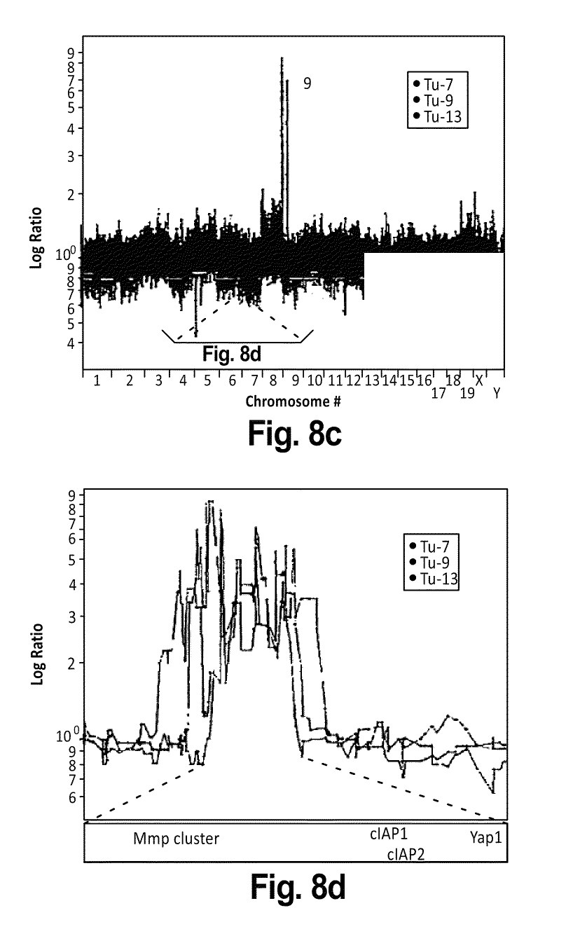

Genomic representations were produced from DNA obtained from several murine liver tumors and from normal mice tissue of the same genetic background, fluorescently labeled and hybridized to microarrays. The data derived after scanning was normalized as described (Sebat et al. “Large-scale copy number polymorphism in the human genome.” Science 305, 525-528. (2004)). Even though all tumors were derived from cells harboring two defined genetic lesions, some displayed a small number of focal copy number alterations. For example, a ras-expressing tumor harbored two focal amplifications on chromosome 15 (FIG. 8A), including a 250 Kb amplicon that contains Rnf19 and a 2 Mb amplicon containing c-myc (FIG. 8B). While Rnf19 has not been previously linked to tumorigenesis, c-myc amplification is a common event in human liver cancer and, furthermore, c-myc cooperates with oncogenic ras in transgenic models of HCC (Sandgren et al. “Oncogene-induced liver neoplasia in transgenic mice.” Oncogene 4, 715-724.(1989)). That a mutation affecting an established liver oncogene can occur spontaneously in these tumors underscores the relevance of the model of the present invention, and suggests that further analyses would reveal other genes involved in human cancer.

Recurrent Amplification of Chromosome 9qA1in Myc-Expressing HCCs

ROMA was also performed on seven independently derived Myc-expressing HCCs, and identified a focal amplicon on mouse chromosome 9qA1 in three of these tumors (FIG. 8C). As shown in a high resolution view (FIG. 8D), the minimal overlapping region was approximately 1 Mb and contained genes encoding for several matrix metalloproteinases (MMPs), Yap1, c1AP1 (Birc2), and c1AP2 (Birc3) as annotated in the UCSC genome browser. An EST to Porimin also maps to this region. Amplification of this region was confirmed by genomic Q-PCR using a probe targeting the middle of the 9qA1 amplicon within the c1AP1 gene, a technique that also identified the 9qA1 amplicon in a fourth myc-expressing tumor (data not shown). Remarkably, 9qA1 was never found amplified in 15 other liver carcinomas expressing either Ras or Akt. These observations suggest that at least one of the genes in the 9qA1 region cooperates with myc and p53 loss to promote hepatocarcinogenesis.

### Comparative Oncogenomics Reveals Lesions in Common Between Murine and Human Cancers

In parallel to the analysis of murine HCCs, ROMA was conducted on 25 human HCC samples. These human tumors showed more complex alterations than the murine HCCs, yet we were able to detect copy number alterations affecting genes previously linked to HCC. For example, three tumors had a chromosome 11 amplification containing CCND1 (cyclin D1), two had a chromosome 7 amplification containing c-MET, and one had a deletion of chromosome 9 harboring the CDKN2A (INK4a/ARF) locus (data not shown).

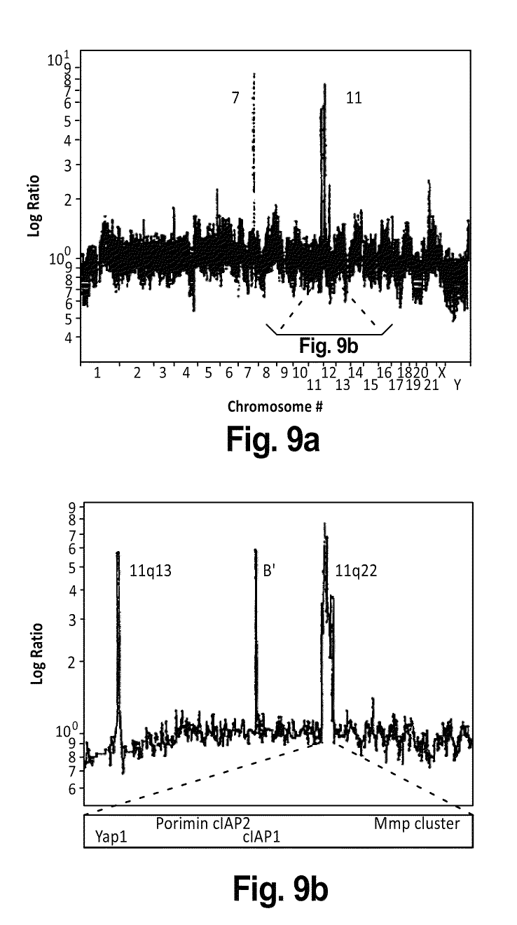

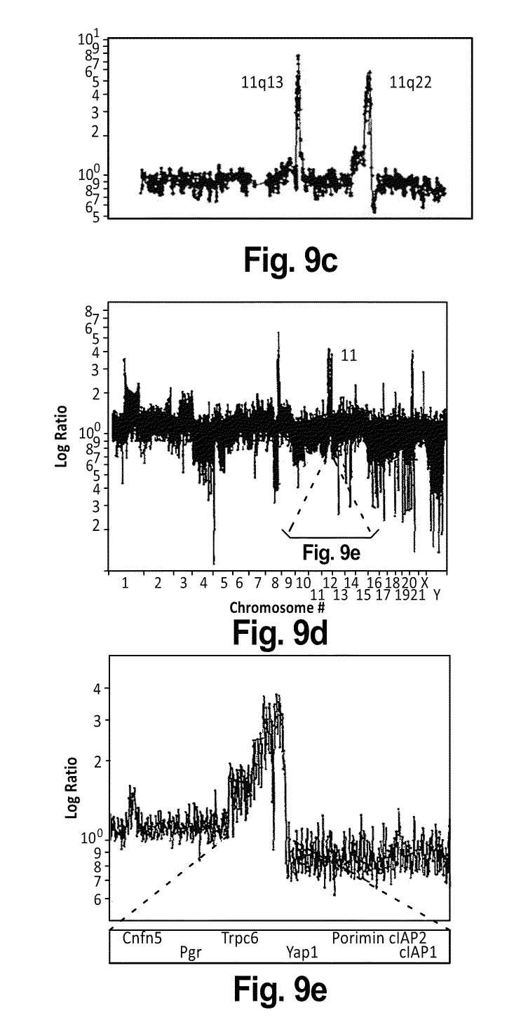

FIG. 9A shows a genome-wide profile of a liver tumor with a c-MET amplification (left peak) on chromosome 7, and three sharply delineated amplifications on chromosome 11 (FIG. 9B), including CCND1, B′ (containing no known genes), and 11q22. Only focal gains or losses <5 MB were included in the analysis. Interestingly, the amplified region of human chromosome 11q22 is syntenic to mouse 9qA1, the region amplified in murine HCCs described above. Through the use of ROMA and/or genomic quantitative PCR to the c1AP1 and c1AP2 loci, we detected this same amplicon in a panel of human esophageal cancers (FIG. 9C, 4 of 53 tested; data not shown), indicating that it occurred in gastrointestinal malignancies derived from developmentally related organs. Much like the chromosome 9 amplicon in murine HCCs, the boundaries of this 11q22 amplicon include genes encoding several matrix metalloproteinases, Porimin, Yap1, c1AP1 and c1AP2.

The human 11q22 amplicon has previously been observed at low frequency in other human cancers, although no driver gene has been decisively identified. While it represents only one of many low frequency events in these tumors, our cross-species comparison suggests that a gene(s) in the amplified region is crucial for tumorigenesis in certain contexts. In an attempt to further narrow down potential candidates, a series of other cancers that were previously analyzed by ROMA were examined to identify the minimal region of overlap. FIGS. 9D and 9E show an example of an ovarian carcinoma harboring an 11q22 amplicon that was particularly informative: although this tumor is genomically unstable, the MMPs are excluded from this 11 q22 amplification suggesting that overexpression of this cluster is not the key selected driving event. Therefore, the genes contained within the epicenter of the amplicon are Porimin, Yap1, c1AP1 and c1AP2.

c1AP1 is Consistently Overexpressed in Tumors Harboring the Murine 9qA1 and Human 11q22 Amplicons

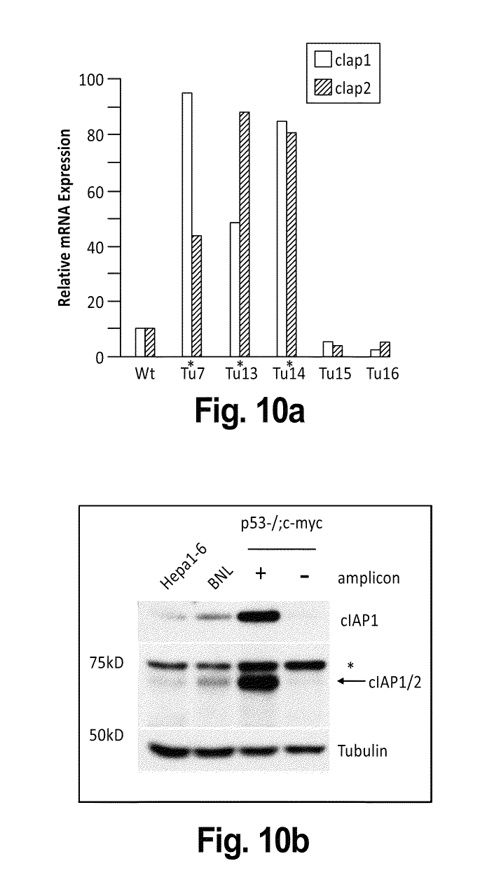

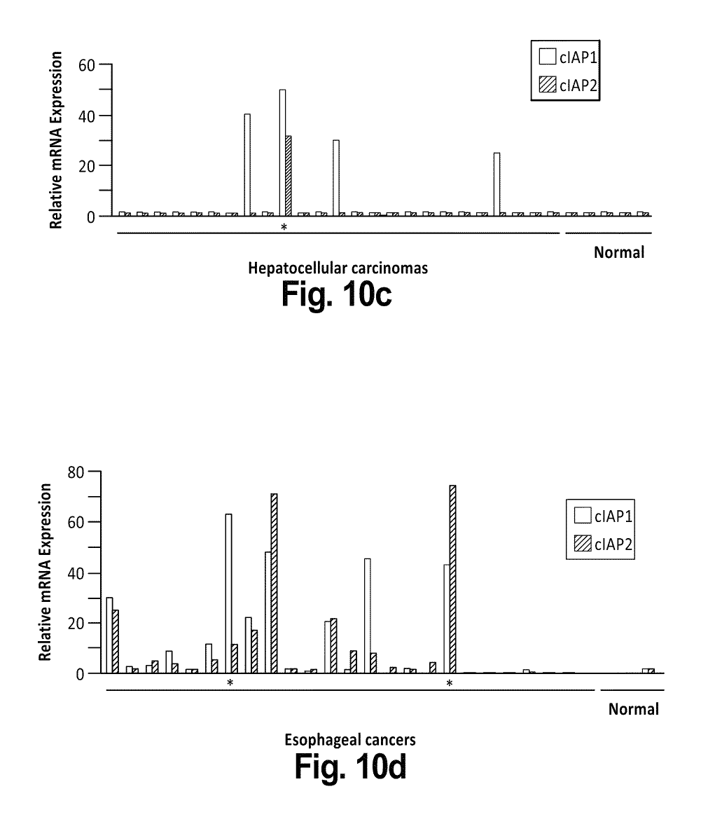

One criterion for establishing whether a gene in an amplicon might contribute to tumorigenesis is that it shows elevated expression in the tumor. RT-Q-PCR analysis was conducted on a series of tumors to examine which genes in the mouse 9qA1 and human 11q22 amplicons were consistently overexpressed. The murine p53−/−; myc HCCs that contain the 9qA1 amplicon consistently overexpressed c1AP1 and c1AP2 mRNA, and c1AP1 protein (FIGS. 10A and B). These genes were not upregulated in tumors without the amplicon. Both genes were overexpressed in the human HCC and esophageal tumors harboring the 11q22 amplicon, but also in a substantial number of tumors without c1AP1/c1AP2 copy number elevation (FIGS. 10C and D; 4 of 25 HCC; 15 of 50 esophageal). Interestingly, c1AP1 was the only c1AP overexpressed in human HCCs without 11q22 amplification (FIG. 10C), hinting that this gene might have a particularly important role in this disease. Based on these analyses, c1AP1 is a likely driver gene in liver cancer.

c1AP1 has Oncogenic Properties

Inhibitor of apoptosis (IAP) proteins were originally identified in baculovirus because of their potential to inhibit cell death of infected cells. Similar to their viral counterparts, overexpression of cellular 1APs can inhibit apoptosis induced by different stimuli. Although 1APs have been shown to bind and inhibit caspases, it is controversial as to whether they are important regulators of apoptosis in mammalian cells. Furthermore, although indirect evidence point towards 1APs playing a role in oncogenesis (Wright & Duckett. “Reawakening the cellular death program in neoplasia through the therapeutic blockade of IAP function.” J. Clin. Invest 115, 2673-2678.(2005)), there remains no direct evidence that these genes actively contribute to tumor initiation or maintenance.

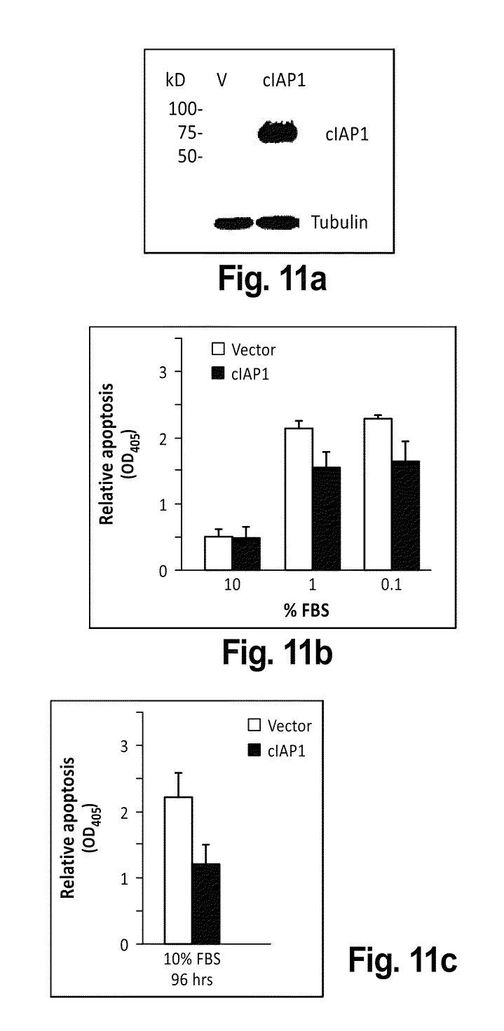

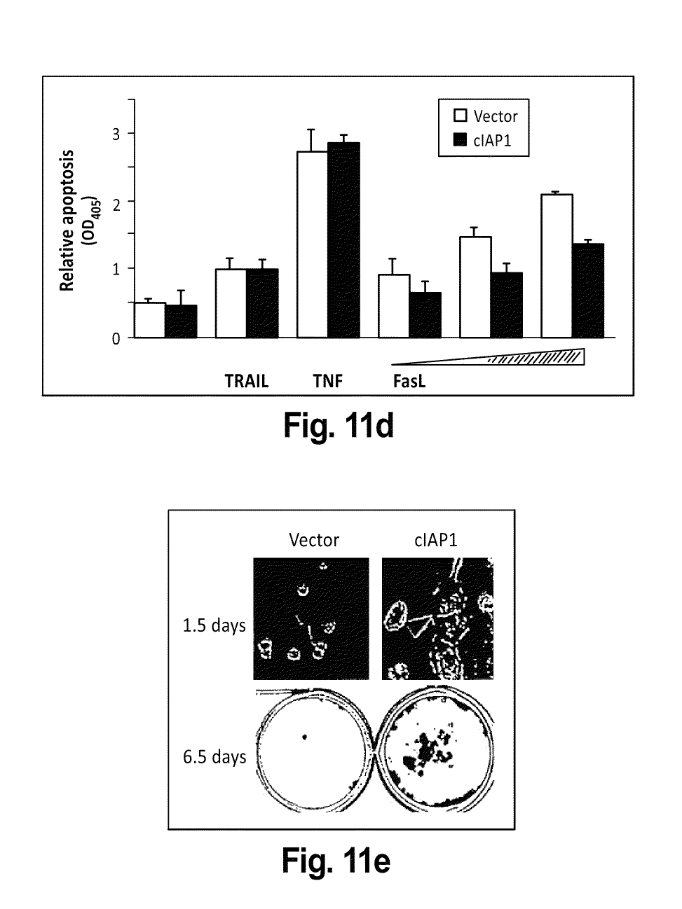

A significant advantage of profiling the genomes of defined murine tumors is that candidate genes can be validated in the genetic context, in which the mutation spontaneously arose during tumorigenesis. Our studies identified the 9qA1 amplicon in tumors derived from p53−/− hepatoblasts expressing Myc but not in other configurations, suggesting these cells would be ideal for evaluating the oncogenic properties of c1AP1. Therefore, p53−/−; myc liver progenitor cells expressing c1AP1 or a control vector were produced using retroviral mediated gene transfer, and the resulting cell populations were examined for transgene expression (FIG. 11A) and subjected to different apoptotic triggers. In this cell type, c1AP1 overexpression conferred a modest protection from growth factor withdrawal and spontaneous cell death at confluence (FIGS. 11B and 11C). Surprisingly, c1AP1 had no effect on apoptosis induced by the death ligands TRAIL and TNFa (FIG. 11D), although it did confer substantial short and long-term protection from Fas-mediated apoptosis (FIGS. 11D and E). Thus, c1AP1 can suppress apoptosis in murine hepatoblasts in vitro.

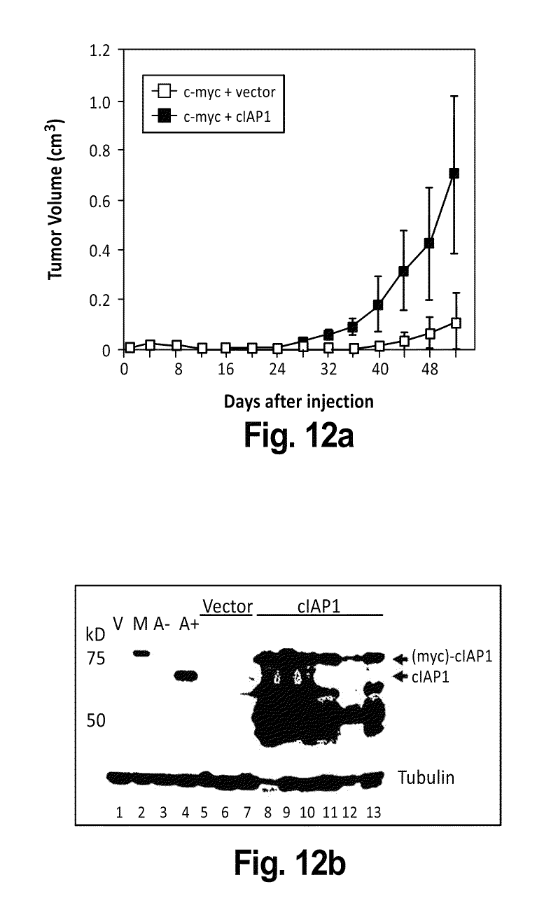

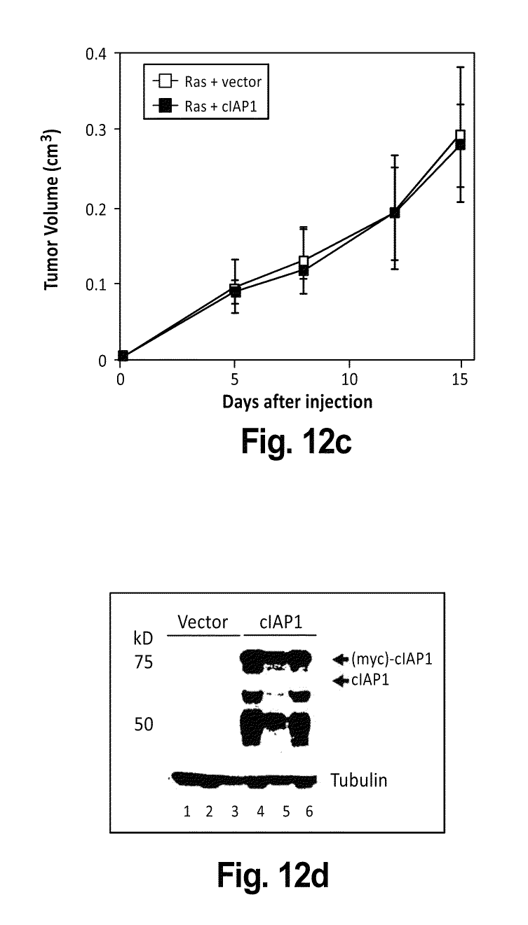

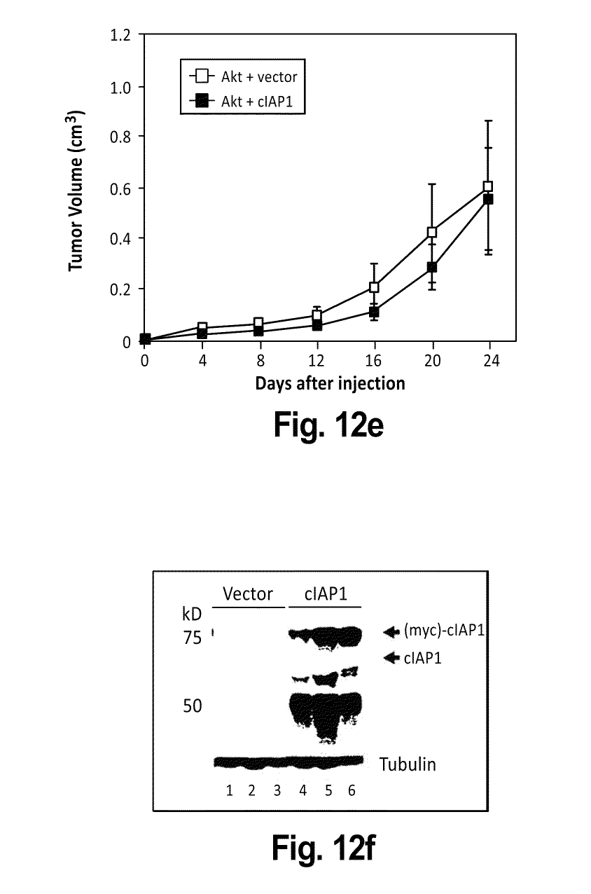

To determine whether c1AP1 could function as an oncogene in vivo, the hepatoblast cultures described above were injected subcutaneously into nude mice to facilitate precise measurement of tumor growth. c1AP1 overexpression significantly accelerated the growth of p53−/− hepatoblasts expressing Myc (FIG. 12A) (onset time of 24±2.3 for myc+c1AP1 vs. 45±12.2 for myc+vector (p=0.02)). The tumors displayed the histopathology of moderately well to poorly differentiated HCC (data not shown) and stably overexpressed the c1AP1 protein at high levels (FIG. 12B, compare lane 2 to lanes 7-13). Also present were low molecular weight forms of c1AP1, consistent with the susceptibility of this protein to proteolytic degradation. Interestingly, one control tumor that was harvested at a very small size showed elevated levels of c1AP1 (FIG. 12B, lane 6), suggesting that a subset of these cells had acquired a spontaneous alteration that upregulated the gene.

The ability of c1AP1 to promote tumorigenicity in cooperation with Akt or Ras was also examined. Using the same procedures described above, we produced p53−/− hepatoblasts expressing either Akt or Ras with or without c1AP1. In contrast to the Myc configuration, overexpression of c1AP1 has no impact on the onset or progression of tumors expressing Akt or Ras (FIGS. 12C and E), even though c1AP1 was efficiently expressed (FIGS. 12D and F). Thus, c1AP1 is selectively oncogenic in the genetic context where its amplification occurs.

c1AP1 and c1AP2 are Required for Rapid Tumor Growth

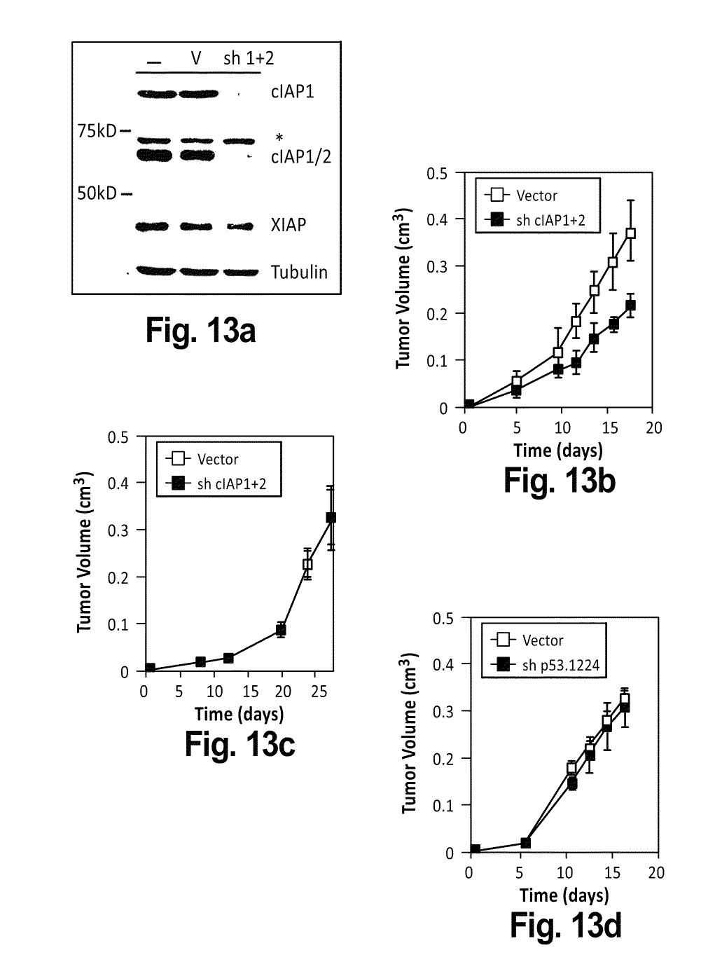

The above data demonstrates that c1AP1 can causally contribute to HCC development. To determine whether the c1AP proteins were required to sustain tumor growth, the impact of reducing c1AP levels on the growth of Myc-induced HCCs was examined in vivo. The expression of c1AP1 and c1AP2 was suppressed, since c1AP2 can be upregulated in response to c1AP1 suppression. First, a series of retroviral vectors expressing shRNAs capable of suppressing c1AP1 (hygromycin selectable) and c1AP2 (puromycin selectable) expression by RNA interference were generated. The best performing shRNAs were co-introduced into outgrown Myc-induced HCC cells containing or lacking the 9qA1 amplicon. Using these vectors, there was significant downregulation of endogenous c1AP1/2, as shown by immunoblotting using an antibody directed against c1AP1 or an antibody that cross-reacts with both c1AP1 and c1AP2 (FIG. 13). Some of these cells were also subsequently injected subcutaneously into the flanks of immuno-compromised mice, and tumor growth was assessed by caliper measurement.

In tumors harboring the 9qA1 amplicon, suppression of c1AP1/2 had a marked impact on tumor growth. Thus, tumors expressing c1AP1 and c1AP2 shRNAs showed a reduced growth rate compared to parallel tumors expressing the control vectors (FIG. 13B). By contrast, these same shRNAs had no impact on the growth of an amplicon negative tumor derived from the same genotype (FIG. 13C), suggesting that only cells selected for c1AP overexpression are sensitive to c1AP inhibition. This latter observation also rules out off-target effects of these shRNAs on tumor growth. Consistent with this hypothesis, a p53 shRNA did not inhibit the growth of the p53−/−; myc tumor containing the 9qA1 amplicon (FIG. 13D). Therefore, c1AP1 and 2 are required for the efficient growth of tumors harboring the 9qA1 amplicon and thus may be therapeutic targets in a subset of human cancers.

### Example 2

An integrated oncogenomic approach was undertaken to identify new tumor suppressor genes in hepatocellular carcinoma. MicroRNA based shRNA technology was combined with a progenitor cell derived mouse model of liver cancer to perform in vivo RNA interference screens for new tumor suppressor genes in liver cancer.

### Selecting RNAi Libraries Based on Human Oncogenomic Data

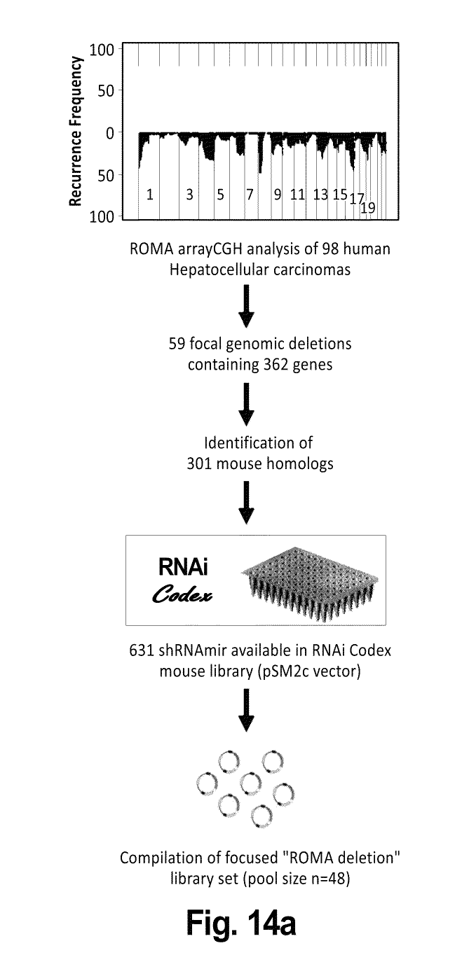

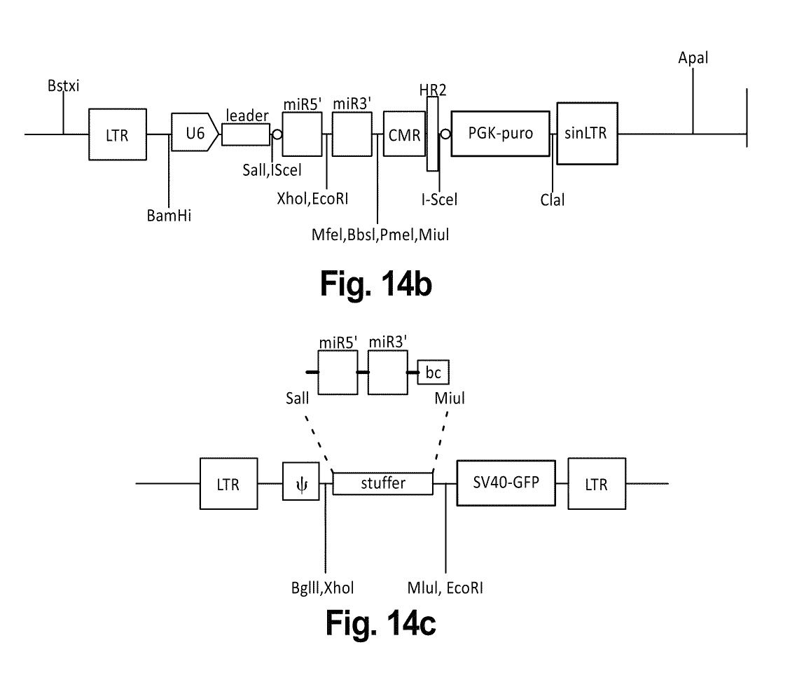

98 human hepatocellular carcinomas of different etiologies were analyzed by Representational Oligonucleotide Microarray (ROMA) analysis, a high resolution array-CGH based platform, in order to identify recurrent focal genomic deletions. 59 focal genomic deletions (<5 MB) were identified, embedding a total of 362 known genes. For 301 of these genes mouse homologs were identified. A total of 631 shRNAmirs targeting these 301 genes were obtained from the RNAi Codex Cold Spring Harbor shRNAmir mouse library. Cultures of pSMc shRNAmir library clones were pooled to assemble low complexity shRNAmir library pools (n=48) (FIG. 14a). To allow for in vivo RNAi screening, SalI/MluI fragments (which contains shRNAmir and a unique barcode sequence for every hairpin sequence) were shuttled from the low complexity pSM2c library pools (FIG. 14b) into an MSCV based retroviral vector which has been optimized for in vivo use (FIG. 14c). In this vector the microRNA based shRNAs are driven by the retroviral LTR promoter, which has been shown to greatly improve knockdown efficiency, even at single copy integration in the genome. Resulting shRNAmir library pools underwent high throughput sequencing to confirm that subcloning of the library pools did not impact their complexity and representation (data not shown).

### RNAi Constructs Targeting APC Tumor Suppressor Gene Induce Liver Carcinoma

It was discovered that embryonic hepatocytes harvested from p53−/− mice are only immortalized but not transformed after transduction with a single copy c-myc transgene. We therefore used ED18 c-myc; p53−/− cells as our test system to perform in vivo RNAi screens for new tumor suppressor genes in hepatocellular carcinoma.

Deregulated Wnt-signaling is prevalent in a high percentage of human hepatocellular carcinomas, mostly due to activating mutations in the beta catenin gene, inactivating mutations in the axin tumor suppressor gene, or promoter hypermethylation of the APC tumor suppressor gene.

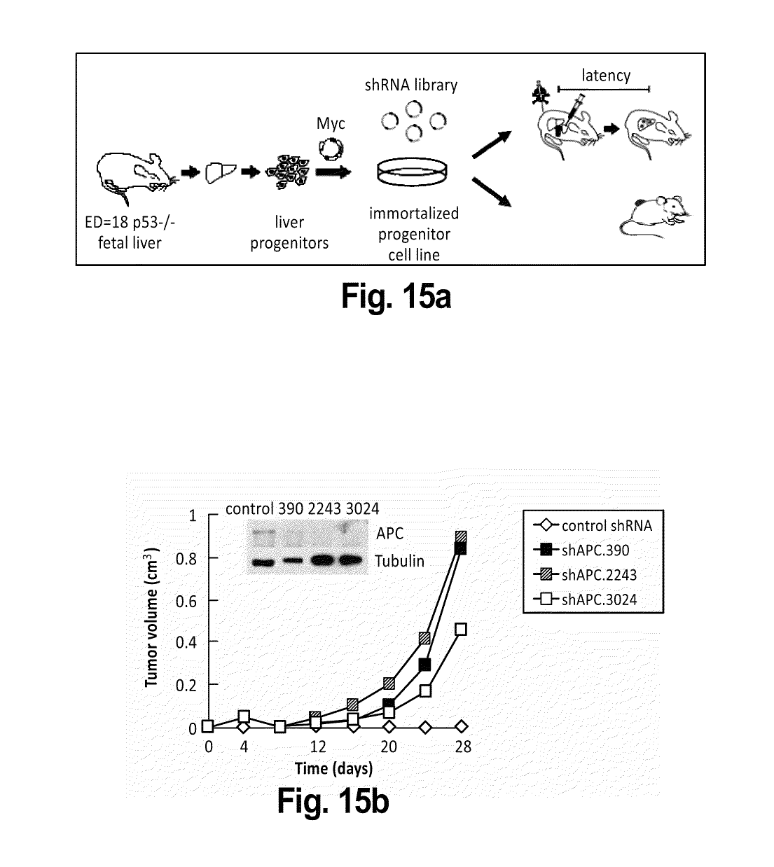

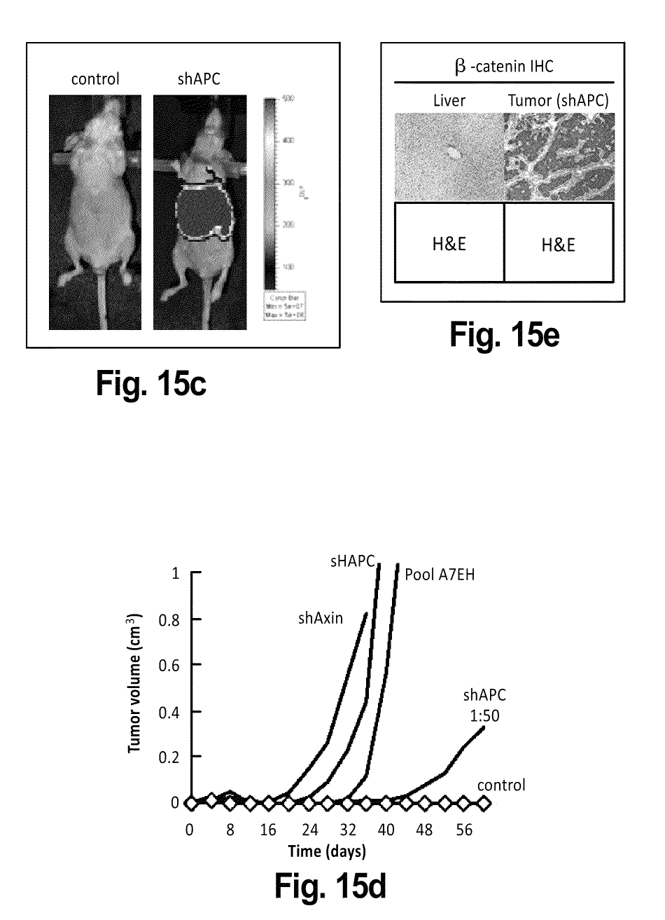

Two independent populations of ED18 c-myc/p53−/− embryonic hepatocytes were generated and transduced with three different shRNAmir targeting the APC tumor suppressor gene or with a control shRNA and transplanted the resulting cell populations either subcutaneously on nude mice or seeded the cells into the livers of recipient mice via intrasplenic injection. While shRNAmir^{APC} transduced cells gave rise to liver carcinomas in both settings (FIGS. 15, a, b), transduction with control shRNAs did not trigger tumor growth of the same cell populations. Intrahepatic tumor growth could easily be followed up by bioluminescence imaging as cell populations were initially established using a luciferase tagged myc transgene. H&E staing of sections from liver carcinomas evolving from the c-myc;p53−/−; shAPC compound mutant cells were classified as aggressive hepatocellular carcinomas, mostly showing a solid, sometimes a pseudoglandular growth pattern (FIG. 15c, top panel). Immunohistochemical analysis revealed a strong nuclear accumulation of beta catenin in these tumors.

An important prerequisite for the conduction of RNAi screens using pooled shRNAs in any biological systems is that positive controls score at a dilution which represents the representation of a single shRNA in a library pool. It was tested whether a 1:50 dilution of a shRNAmir^{APC} would still be sufficient to induce tumor growth in the system described herein. Furthermore, c-myc;p53−/− embryonic hepatocytes were transduced with a low complexity shRNAmir library pool which contains two of the scoring shRNAmir^{APC}. Both experiments resulted in tumor growth, whereas the time to tumor onset was delayed compared to the cells transduced with a pure shRNAmir^{APC} construct (FIG. 15d). In addition to shRNAmirAPC mediated deregulation of Wnt signaling, it was found that down-regulation of the Axin tumor suppressor also resulted in the induction of aggressive hepatocellular carcinomas (FIG. 15d).

### In Vivo RNAi Screening to Identify New Tumor Suppressor Genes in HCC

Using an in vivo system system, and in vivo RNAi screen was performed in order to identify new tumor suppressor genes in hepatocellular carcinoma (HCC).

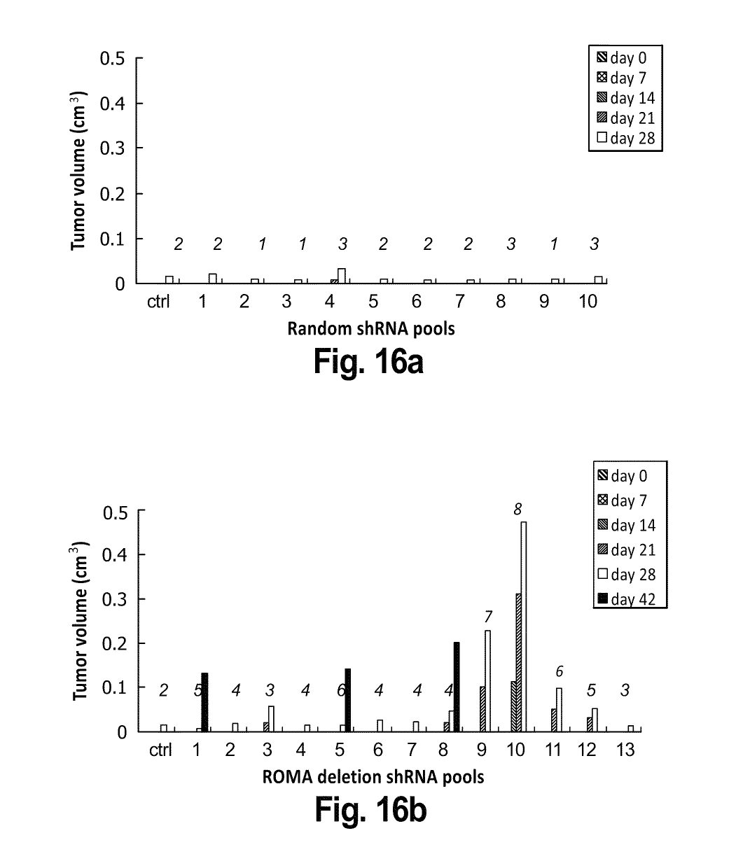

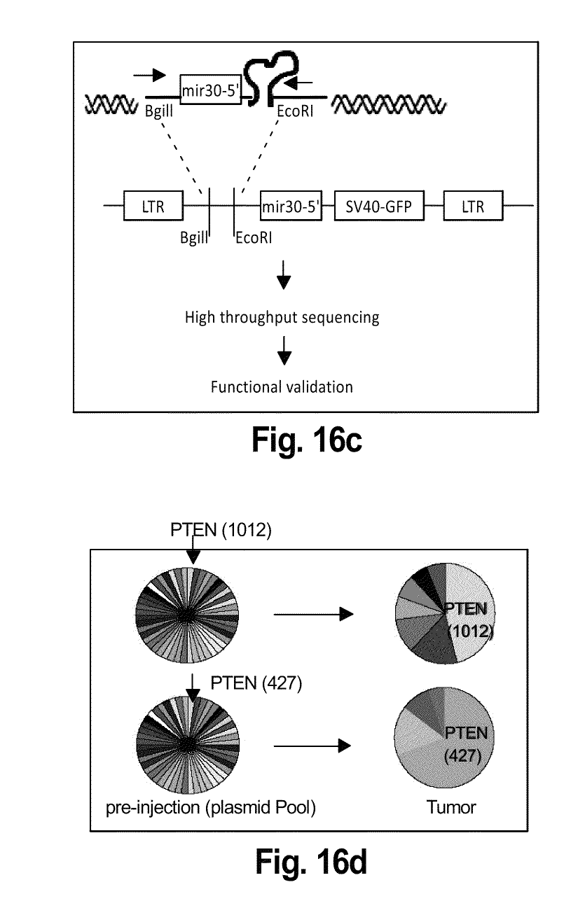

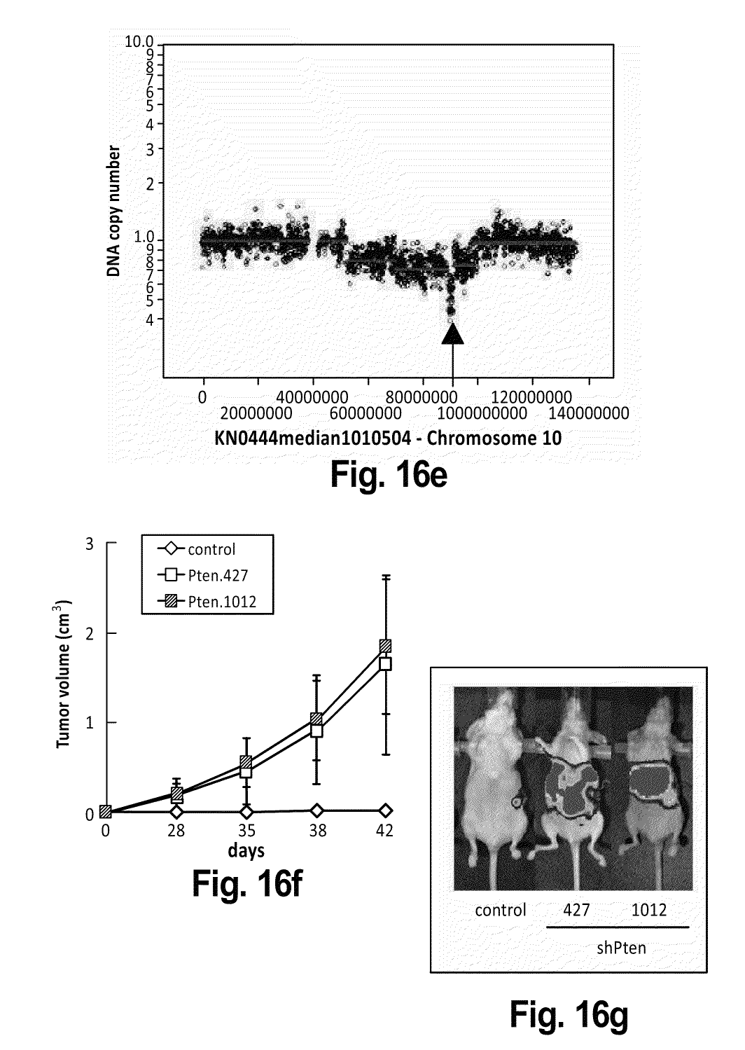

Two independent populations of ED18 c-myc;p53−/− liver progenitor cells were in infected with low complexity shRNAmir pools containing hairpins targeting the genes found in focal deletions of 98 human HCC (see, FIG. 14a, generation of the library pools). While the majority of shRNA pools from this focused library evertually triggered tumor growth, six pools were found that triggerd the growth of aggressive HCCs as early as 14-21 days after injection of the cells (FIG. 16a). Importantly, only few tumors developed after long latency when the same c-myc;p53−/− cell populations were in parallel transduced with ten random shRNAmir pools (taken from a genome wide murine shRNAmir library) of the same pool size (n=48) (FIG. 16b).

To identify scoring shRNAmirs which cooperated with c-myc and p53 deficiency during tumorigenesis, genomic DNA was isolated from GFP-positive tumor nodules. Via PCR a fragment was amplified containing the mir30-5′ flanking region as well as the unique shRNA sequence (FIG. 16c). PCR products were digested with BglII and EcoRI and cloned directionally into a recipient vector which contains the mir30-3′ flanking region. The resulting vectors, containing the full shRNAmir cassette, were submitted for high throughput sequencing. At least hundred sequence reads were run per tumor, whereas at minimum three tumors from each pool (two independent experiments) were sequenced.

Down-Regulation of the PTEN Tumor Suppressor Cooperates with myc and p53-Deficiency During Hepatocarcinogenesis

shRNAmir representation was first analyzed in tumors which only showed moderately accelerated tumor growth. Interestingly, it was found that tumors derived from two independent shRNAmir pools showed strong enrichment for two different hairpins targeting the PTEN tumor suppressor (FIG. 16d). Pie charts showing representative sequencing results from murine HCCs are shown in FIG. 16d (right panel). shRNAmirPTEN-1012 represented about 50% of all shRNAmir in the tumor, while shRNAmirPTEN-427 was even found in >40% of all sequence reads (shRNAmir representation in the low complexity shRNAmir library pool is symbolized by the pie chart in the left panel of FIG. 16d).

The hitherto results suggest that down-regulation of the PTEN tumor suppressor cooperates with myc and p53-deficiency during hepatocarcinogenesis. The data are in accordance with published results showing activating mutations in Pi3k protein in HCCs, and biochemical activation of the Akt kinase in 40% of all human HCCs. The ROMA analyses identified PTEN deletion in a total of four human hepatocellular carcinomas, one of them showing a focal deletion <5 MB (inclusion criterion for the focused shRNAmir library) and three additional ones with broader deletions >5 MB. A ROMA plot showing a focal, PTEN containing deletion on chromosome 10 in a human HCC is shown in FIG. 16e.

It was then investigated whether the scoring shRNAmir that were identified in our in vivo RNAi screen could be functionally validated in the system. Both shRNAmirPTEN from the screen significantly knocked down PTEN protein levels. When infectied into myc; p53−/− cells, both shRNAmir triggered the growth of liver carcinomas, while controls shRNAmir did not induce subcutaneous tumor growth in the same observation period (FIG. 16f). When the same shRNAmirPTEN transduced cell populations were seeded into the livers of recipient mice by intrasplenic injection, they also gave rise intrahepatic HCCs, as shown by bioluminescence imaging 4 weeks after seeding of the cells (FIG. 16g).

Identification of New Genes Associated with Tumorigenesis

shRNA representation was systematically analyzed in all tumors, in particular those which showed fastest tumor growth.

A total of 3900 sequence reads were applied to 39 tumors induced by the 13 low complexity shRNAmir pools. Table 1 lists all shRNAs found enriched to >5% of the total sequencing reads in each given pool.

A total of 36 candidate shRNAs enriched to >5% in tumors induced by the respective pool were found. Based on the enrichment factor, the two identified shRNAs targeting PTEN represent the 5^{th} and 15^{th} best scoring shRNAs, respectively.

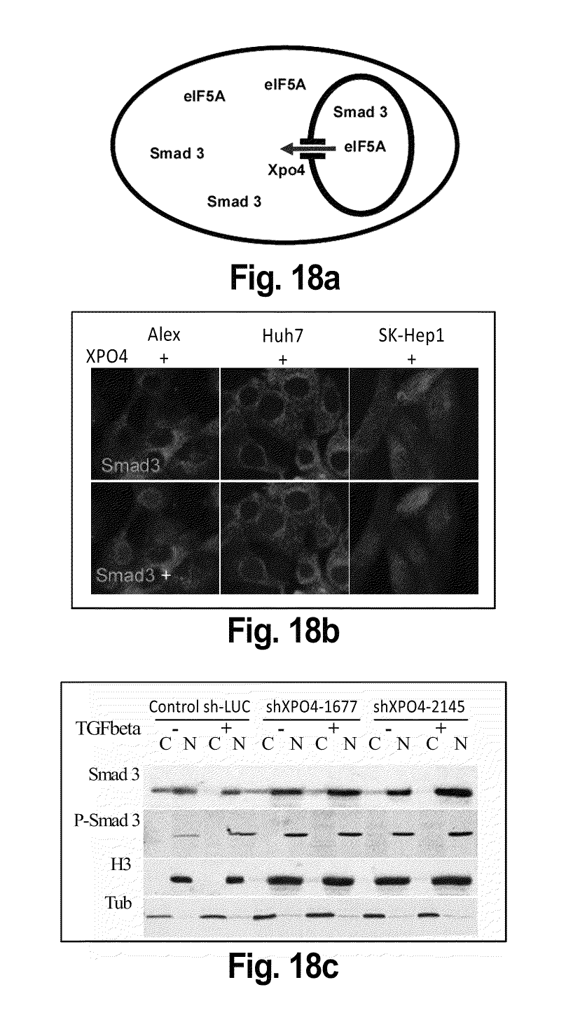

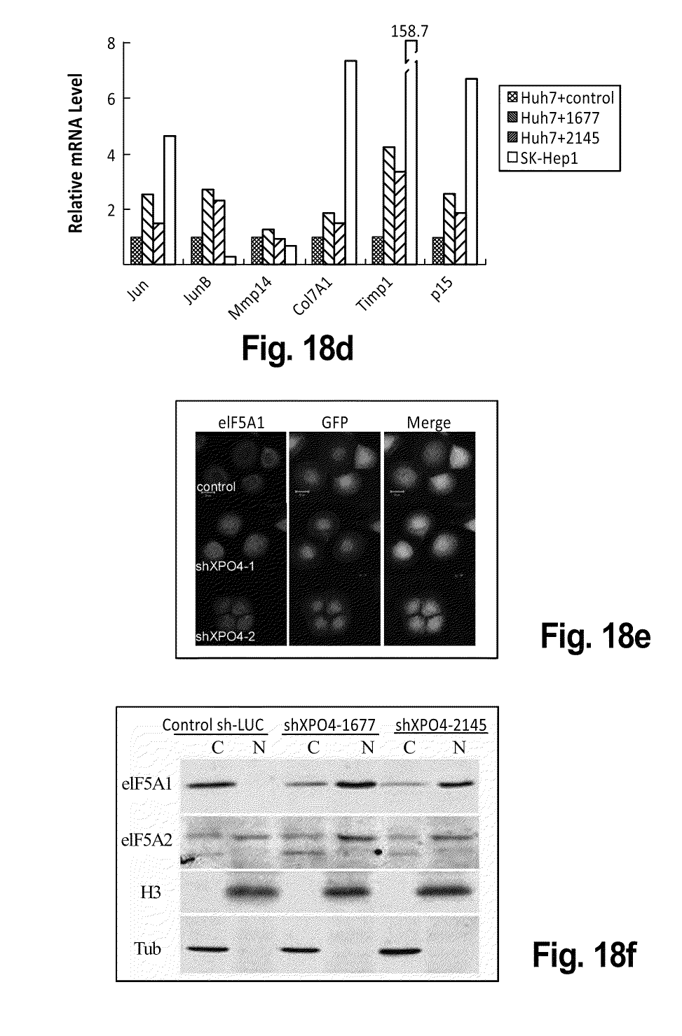

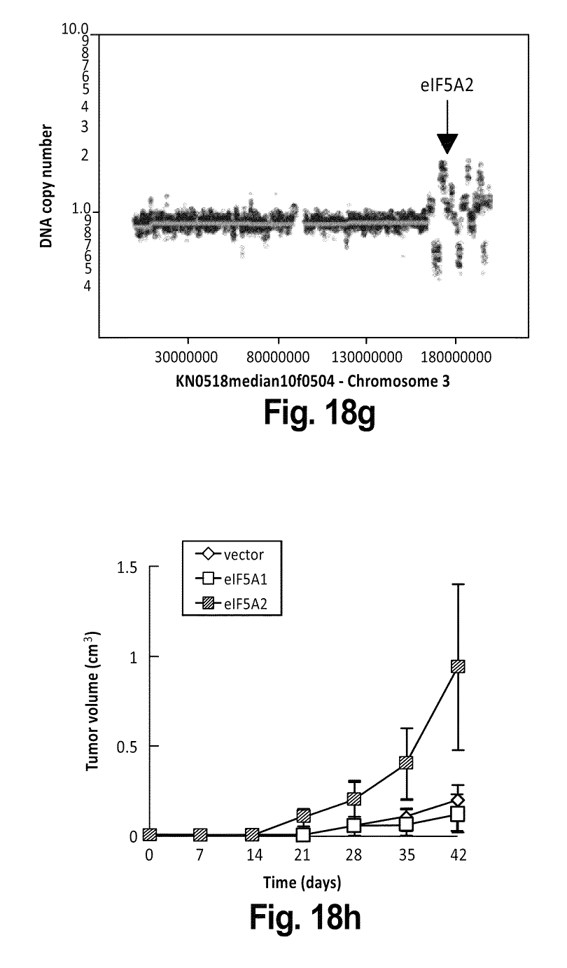

The top scoring shRNA targeted Xpo4, a member of the importin-β superfamily. It has been shown that Xpo4 serves as a nuclear exporter for eIF5A1 and Smad3 but loss of Xpo4 expression has never been linked to cancer. Other genes targeted by the top five scoring shRNAs are FgF6, Wdr49 and Armcx2. Interestingly, none of these genes have been linked to tumorigenesis before. FIG. 18A shows pie charts which reveal the shRNA representation in five representative tumors. As shown, the top scoring shRNA targeting Xpo4 is enriched to >75 of all sequencing reads of the respective tumor. Other candidates like Cx40.1, Fst15 and Gm123 show significant enrichment, however to a lesser extend than Xpo4. A systematic analysis of the sequencing results from all tumors derived from a respective pool is shown in Table 2.

Next it was determined which candidates should be followed up in functional validation experiments. Candidates were chosen based on two criteria: i) overall enrichment of an shRNA in tumors triggered by a particular shRNA pool (high enrichment coefficient) and ii) detection of more than one scoring shRNA against a target. Based on these criteria a total of 17 shRNAs went into functional validation experiments using the same setup as used for the conduction of the initial screen (including a shRNA targeting the PTEN tumor suppressor, which already had been validated before).

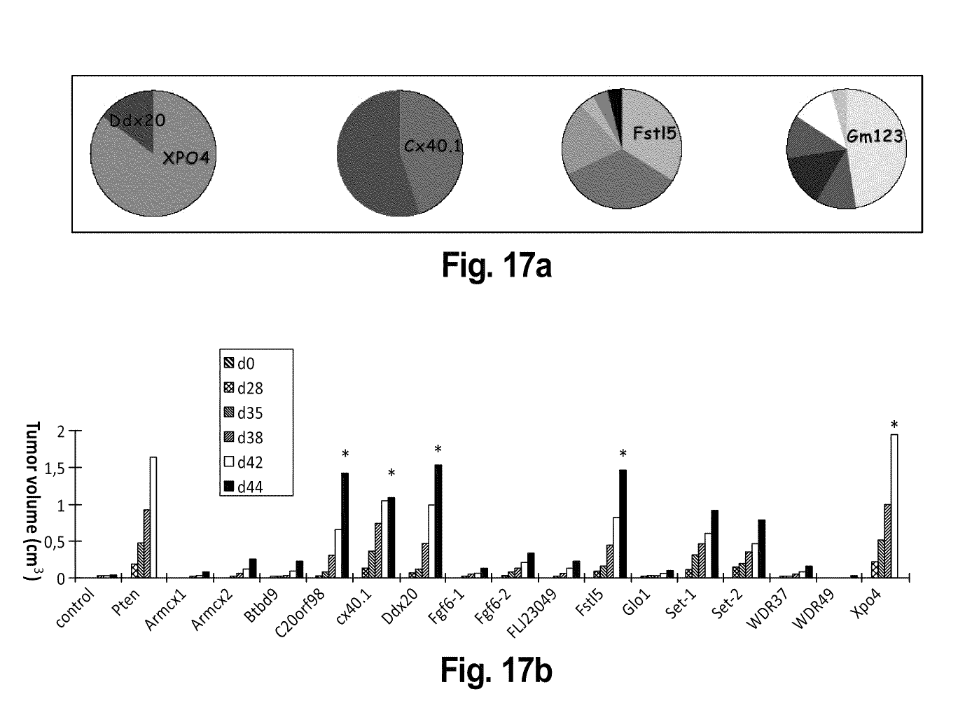

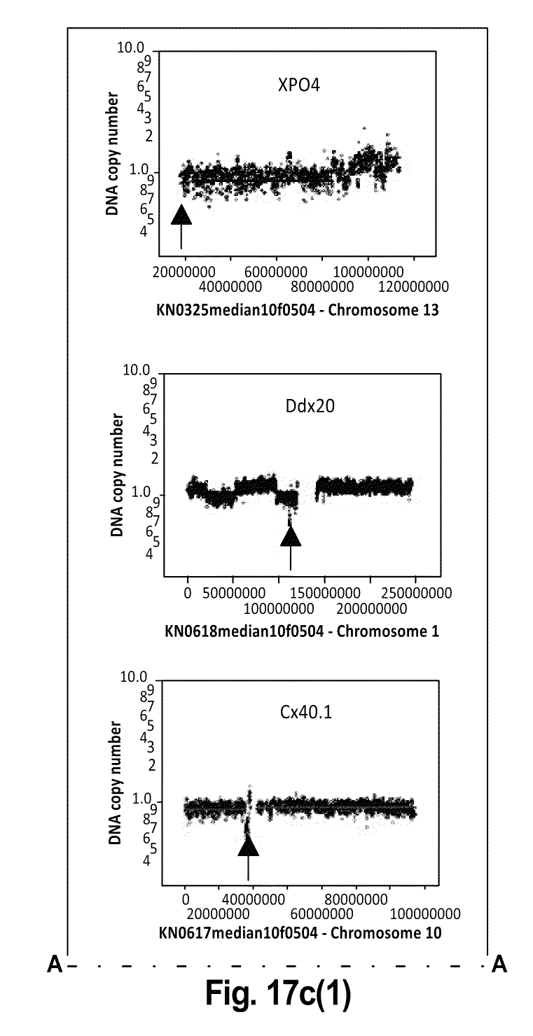

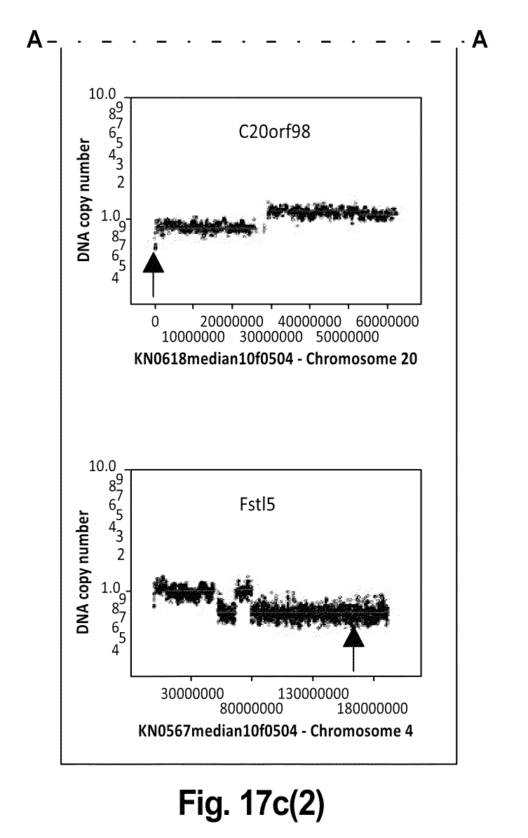

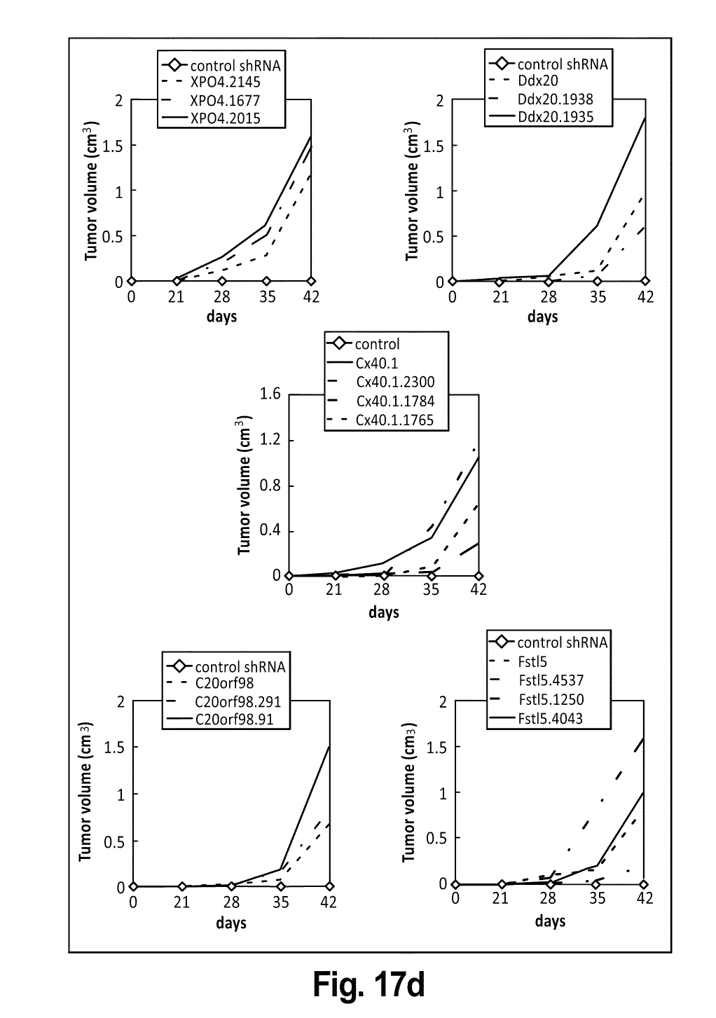

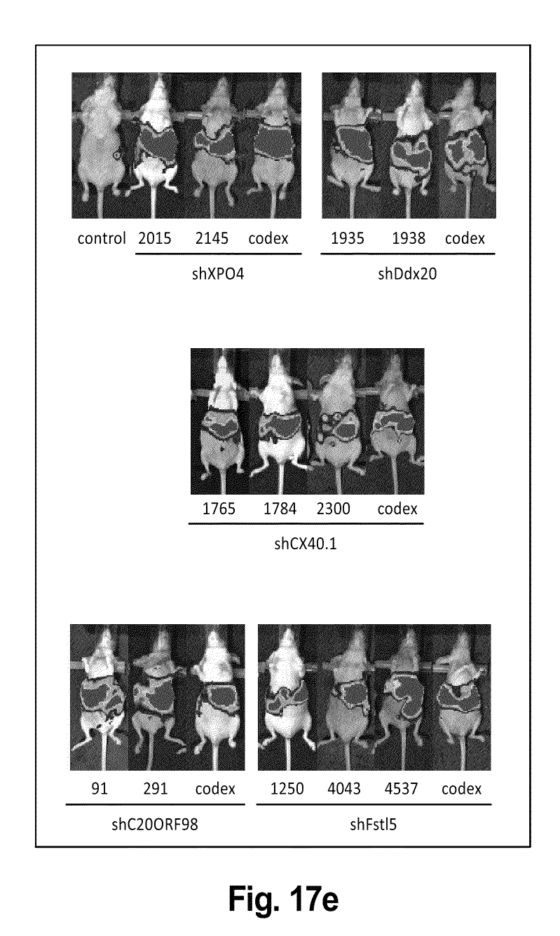

As shown in FIG. 17B, in addition to the PTEN shRNA, five other candidate shRNAs were capable of accelerating tumor growth of subcutaneous liver carcinomas (C20orf98, Cx40.1, Ddx20, Fst15 and Xpo4). Interestingly, the top scoring candidate Xpo4 showed the most rapid acceleration of tumor growth. FIG. 17C reveals the focal genomic deletions in human HCCs (high resolution ROMA profiles) which are emdbedding the identified candidate genes. Of note, several of the 17 tested shRNAs showed acceleration of tumor growth above background, albeit less pronounced than the top scoring genes.

Using the biopred webtool de novo additional shRNAs were generated targeting the top scoring candidate genes. In addition to the scoring shRNAs libraries, the RNAi Codex library, at least two additional shRNAs were generated (FIGS. 17, D and E). These shRNAs were infected into p53−/−; myc liver progenitor cells side by side with the original Codex shRNAs and cell populations were either injected subcutaneously (FIG. 17D) or seeded into the livers of recipient mice by intrasplenic injection (FIG. 17E). Importantly, all shRNAs that were tested accelerated tumor growth with an efficiency similar or greater than the Codex shRNAs (FIGS. 17, D, E).

### Functional Characterization of Xpo4

Studies were then undertaken to gain mechanistic insight how loss of Xpo4, the top scoring candidate in the RNAi screen, promotes tumorigenesis. To date, two substrates for Xpo4 have been identified: Smad3, (give background) and eIF5A1 (see, FIG. 18A).

To address whether Xpo4 expression correlates with the amount of nuclear total and phospho-Smad3, murine hepatoma cells (p53−/−; myc) were used which were either infected with a control hairpin (shRNAmir luciferase) or two different hairpins targeting Xpo4. As shown by western blot in FIG. 18C, knockdown of Xpo4 with both shRNAmir leads to increased nuclear levels for total smad 3 and the transcriptionally active phosphor-Smad3. Results from western blot were confirmed by immunofluorescence (data not shown).

The correlation between Xpo4 status and Smad3 localization in human hepatoma cells was then analyzed. SK-Hep-1 human hepatoma cells harbor a homozygous deletion of Xpo4 (data not shown). Accordingly, Xpo4 mRNA levels could not be detected in SK-Hep-1 cells, while significant levels of Xpo4 mRNA could be detected by RT-Q-PCR in Huh7 and Alexander human hepatoma cells which display wild type status for the Xpo4 gene. While Huh7 and Alex hepatoma cells show a prominent cytoplasmic staining and no staining for Smad3 in the nucleus when analyzed by immunofluorescence, SK-Hep1 cells display a homogenous distribution of Smad3 protein in cytoplasm and nucleus.

TGF-beta signaling has been shown to be a double edged sword in tumorigenesis. Stimulation of TGF-beta receptor results in nuclear accumulation of Smad3 which, together with other transcription factor leads to the trans-activation of TGF-beta target genes.

To test whether increased nuclear levels of Smad3/p-Smad3 indeed lead to increased transcription of TGF-beta target genes, RT-Q-PCR analysis was performed for classical TGF-beta/Smad3 target genes. It was observed that RNAi mediated reduction of Xpo4 gene expression results in increased mRNA levels for the pivotal TGF-beta target genes Jun, JunB, Mmp14, Co17A1, Timp1 and p15 (data not shown). As it is well established that increased TGF-beta signaling increases tumor progression, invasion and metastasis in later stager of tumor development, the results suggest that, at least in part, reduction of Xpo4 expression is pro-tumorigenic by inducing TGF-beta target genes.

In addition to Smad3, it has been shown that elongation initiation factor 5A1 (eIF5A1) is a substrate for Xpo4. To confirm nuclear export of eIF5A1 by Xpo4 in hepatoma cells, immunofluorescence experiments were performed against eIF5A1 in murine hepatoma cells with or without shRNAmir mediated knockdown of Xpo4. While cells infected with the control shRNA showed almost no nuclear staining of eIF5A1, upon RNAi mediated knockdown of Xpo4 we detected a strong nuclear enrichment of eIF5A1. Results could be confirmed by western blot using cytoplasmic and nuclear protein preparations.

In addition to eIF5A1, a second eIF5 isoform has been identified, designated eIF5A2. Under normal conditions expression of eIF5A isoforms is regulated in a tissue specific manner. Normal tissues usually shown eIFA1 expression but lack eIF5A2 expression. However, increased eIF5A2 expression has been found in various cancer types, making eIF5A2 a candidate oncogene.

Interestingly, among the 98 human analyzed human HCCs, a tumor which displayed a focal amplicon for eIF5A2 was found, thus further supporting the idea of eIF5A2 as a candidate oncogene downstream of Xpo4. It was investigated whether loss of Xpo4 would affect cellular localization of eIF5A2. While the commercially available antibodies did not allow analysis of cellular localization of eIF5A2 by immunofluorescence, using immunoblot analysis on nuclear and cytoplasmic extracts it was found that Xpo4 knockdown leads to a moderate but significant nuclear retention of eIF5A2.

Although not wishing to be bound by particular theories, a possible explanation to link the mislocalization of eIF5A1/2 with tumorigenesis is that, mislocalization of eIf5A1 and A2 via a feedback loop leads to a compensatory increase of A1 and A2, the second of which promotes tumorigenesis. In accordance with this model, we found a strong upregulation of eIF5A1 and eIF5A2 mRNA levels in murine and human hepatoma cell upon Xpo4 knockdown (data not shown). Interestingly, increased transcript levels only resulted in higher protein levels in the case eIF5A2 but not A1 (data not shown).

The data suggest that loss of Xpo4 increases hepatocarcinigenesis by upregulation of eIF5A2 expression. To address whether increased expression of eIF5A1 or A2 can increase hepatocarcinogenesis in vivo, p53−/−; myc liver progenitor cells were infected with retroviruses encoding eIF5A1 or eIF5A2. As shown in FIG. 18h, it was found that enforced overexpression of eIF5A2 but not A1 increases tumorigenesis of p53−/−; myc liver progenitor cells. Thus confirming eIF5A2 as an oncogene in hepatocarcinogenesis.

### Example 3

### Representational Oligonucleotide Microarray Analysis (ROMA)

Human tumor samples were obtained. The ROMA array-CGH method enables genome-wide profiling of DNA copy number at high resolution (Lucito et al., 2003, Genome Res. 13: 2291-2305). This method was utilized to study gene-dosage alterations in human HCC. A total of 86 HCC samples from three different sources, together with 12 liver cancer cell lines, were analyzed. Microarray measurements were converted to copy number estimates using a segmentation algorithm based on Kolmogorov-Smirnov statistics. In order to restrict subsequent analysis to cancer-associated somatic genetic events, an automated procedure was utilized to mask common germline copy number variations (Lucito et al., 2003). Next, an automated method similar to the MCR method developed by Tonon et al. (2005, Proc. Natl. Acad. Sci. U.S.A. 102: 9625-9630) was utilized to catalog homozygous and other focal deletions (segmented DNA copy number<=0.75 and size<=20 Mb) and to determine the region of common overlap. This analysis detected a total of 130 focally deleted loci, 44 of which were recurrent. The total number of genes within these loci was 3,503. In order to reduce the number of genes, the deletions by size (<=2.6 Mb) and derived a subset of 58 deleted loci containing 362 genes (Table 3).

### Calculation of Gene Deletion Frequencies.

The set of 25,245 human genes from autosomal chromosomes found in NCBI Entrez Gene database (build 35.1) was used after processing so that overlapping genes on each strand were merged, reducing the total gene number to 24,719. Genes were assigned to a given genome segment based on their transcriptional start site. To determine whether or not a given genome segment was deleted in a tumor, those that were near the ground-state were determined by applying a Gaussian mixture model based clustering algorithm to the median copy number ratios. The ground-state eas defined by the smallest group of clusters whose centers were closest to 1 and whose segments comprised over 50% of the autosomal genome. All the deletion segments located outside this ground state with probability of 0.95 or higher were deemed significant. Wherever significant deletions formed a contiguous chain, they were sorted into tiers based on the median value of each segment. The tiers were formed by first selecting a group of seed segments in the chain with a maximal total length subject to a condition that any two segment medians in the group are different with 95% confidence, then assigning each segment to the tier with the closest seed median. The tiers in a chain were numbered in the order of decreasing seed median. Every genomic position in a profile was thus assigned a tier number, defined to be 0 in the absence of a deletion. This procedure, for example, would assign a tier number of “one” to a hemizygous loss from a diploid genome, and a tier number of “two” for a homozygous loss, provided that two non-coincident deletions were in fact observed contiguously in the genome. The counts are mean tier numbers per profile as a function of the absolute genomic position. Further technical details will be described in a manuscript in preparation (A.K. et al., in preparation).

shRNA Library Cloning and Vector Construction.

miR30 design shRNAs were subcloned from the pSM2 library vector into an MSCV-SV40-GFP recipient vector in pool sizes of 48. Maintenance of complexity was verified by sequencing. The coding regions of human EIF5A1 and EIF5A2 were PCR cloned from pCMVsport6 (Open Biosystems) into MSCV-IRES-GFP with a 6×Myc-tag. Myc was expressed using MSCV retroviral vectors.

### Generation of Immortalized Liver Progenitor Cell Lines.

Isolation, culture, and retroviral infection of murine hepatoblasts were described recently (Zender et al., 2006, Cell 125: 1253-1267; Zender et al., 2005, Cold Spring Harb. Symp. Quant. Biol. 70: 251-261). Liver progenitor cells from ED=18 p53−/− fetal livers were infected with MSCV based retroviruses expressing Myc-IRES-GFP or Myc-IRES-Luciferase and two immortalized cell lines were derived.

### Generation of Liver Carcinomas.

Early-passage immortalized liver progenitor cells were transformed by retroviruses expressing single shRNAs or shRNA pools. 2×10⁶ cells were transplanted into livers of female C57/B6 or NCR nu/nu mice (6-8 weeks of age) by intra-splenic injection, or injected subcutaneously on NCR nu/nu mice. Tumor progression was monitored by abdominal palpation, whole body GFP imaging, and bioluminescence imaging (IVIS system, Xenogen). Tumor volume of subcutaneous tumors was determined by caliper measurement and calculated as 0.52×length×width². Bioluminescence imaging was performed as described by Xue et al., 2007, Nature 445: 656-660.

### Histopathology and Immunohistochemistry

Histopathological evaluation of murine liver carcinomas was performed by an experienced pathologist using paraffin-embedded liver tumor sections stained with Hematoxylin/Eosin (H&E). Anti β-catenin antibody (BD Biosciences, 1:100) staining was performed using standard protocols on paraffin-embedded liver tumor sections.

### Immunoblotting

Fresh tumor tissue or cell pellets were lysed in Laemmli buffer using a tissue homogenizer. Equal amounts of protein (16 μg) were separated on 10% SDS-polyacrylamide gels and transferred to PVDF membranes. Blots were probed with antibodies against APC (Calbiochem, FE9, 1:1000), eIF5A1 (Abcam, Clone EP526Y, #ab32443, 1:1000), eIF5A2 (Novas, #H00056648-M01, 1:1000), Myc-Tag (Abcam, 9E10, 1:1000), Smad3 (Cell Signaling, #9523, 1:1000), phospho-Smad3 (Cell Signalling, #9520P, 1:1000) or Tubulin (Sigma, B-5-1-2, 1:5000).

### RNA Expression Analysis

Murine hepatoma cells or tumors were freshly homogenized in Trizol (GIBCO). RNA was isolated according to the manufacturer's instructions, treated with RNase-free DNase (QIAGEN) and purified with QIAGEN RNeasy columns. Total RNA was converted to cDNA using TaqMan® reverse transcription reagents (Applied Biosystems) and used in qPCR reactions with incorporation of SYBR® Green PCR Master Mix (Applied Biosystems). Each reaction was done in triplicate using gene-specific primers. The expression level of each gene was normalized to β-actin.

### Immunofluorescence Microscopy

Cells were fixed in 4% paraformaldehyde in PBS for 20 minutes and permeabilized with 0.2% Triton X-100 in PBS for 5 minutes. Fixed cells were blocked with 5% goat serum and incubated with primary antibodies for 1 hour, followed by goat-anti-mouse Alexa 568 or goat-anti-rabbit Alexa 488 secondary antibodies for 1 hour with washing in between. Antibodies used were as follows: Smad3 (Cell Signaling, Clone C67H9, #9523, 1:100), EIF5A1 (Abcam, Clone EP526Y, #ab32443, 1:100). Cells were counterstained with 4, 6-diamidino-2-phenylindole (DAPI, Sigma) or TO-PRO3 DNA dye (Molecular Probes) and mounted in VECTASHIELD® anti-fade mountant (Vector Laboratories, Burlingame, Calif.). Microscopy was done using a confocal microscope (Zeiss).

### Nuclear Fractionation

Nuclear fractionation was performed according to Hosking et al. (2007, Mol. Biol. Cell 18: 1918-1927). Western blots were performed using the following antibodies: Smad3 (Cell Signaling #9523S), Phospho-Smad3 (Cell Signaling #9520S), Histone 3 (Santa Cruz Biotechnology, SC8654), Mek-1 (Santa Cruz Biotechnology, SC6250), EIF5A1 (Abnova, #64-153) and EIF5A2 (Abeam, #32443), according to the manufacturer's instructions.

### Tissue Culture and Retroviral Gene Transfer

Retroviral-mediated gene transfer was performed using Phoenix packaging cells as described in Schmitt et al., 2002, Cancer Cell 1: 289-298). Population doubling, BrdU staining, and colony formation assays were performed as described.

Cloning of XPO4 cDNA

Full length human XPO4 cDNA was PCR-amplified from Open Biosystems cDNA clone BC113571 using primer set 5′-CGGATCCTCTCAAGGCACCACCTTC-3′ (BamHI) (SEQ ID NO: 1) and 5′-GTCACGCGTTTATTTTACACAAAGGAGAC-3′ (MluI) (SEQ ID NO: 2) and cloned into an MSCV-PIG vector containing 6×Myc tag and puromycin selectable marker. Six amino acids were included between Myc tag and XPO4 open reading frame.

siRNA Transfection

Human hepatoma cells were tranfected by Dharmacon siRNAs. mRNA was harvested 24 hours post-transfection. MTT assay was performed using CellTiter 96® AQueous Cell Proliferation assay kit (Promega) 48 hours post-transfection. Counting of population doublings, BrdU staining, and colony formation assays were performed as described previously (Zender et al., 2006).

### Primer Sequences for RT-Q-PCR

The following primers for mouse genes were used:

| Eif5a1 | |
|---|---|
| 5′-agggagttgggtactgcttg-3′ | (SEQ ID NO: 3) |
| and | |
| 5′-agggcacaagaccatttacc-3′ | (SEQ ID NO: 4) |
| Eif5a2 | |
| 5′-aggggcttccagcacttat-3′ | (SEQ ID NO: 5) |
| and | |
| 5′-cccagtcttggaagttgaca-3′ | (SEQ ID NO: 6) |
| Fst15 | |
| 5′-atggccattgaatacagcaa-3′ | (SEQ ID NO: 7) |
| and | |
| 5′-accaacggcttcttcctaga-3′ | (SEQ ID NO: 8) |
| Ddx20 | |
| 5′-ggaaggctcctcagacactc-3′ | (SEQ ID NO: 9) |
| and | |
| 5′-ctccacttgagtcggggtat-3′ | (SEQ ID NO: 10) |
| Gjd4 | |
| 5′-gacaggctaacagtggctca-3′ | (SEQ ID NO: 11) |
| and | |
| 5′-tctgggtagccaaggagagt-3′ | (SEQ ID NO: 12) |
| Nrsn2 | |
| 5′-ccagtagccgactacaacca-3′ | (SEQ ID NO: 13) |
| and | |
| 5′-cgatcaagaagaggcagaca-3′ | (SEQ ID NO: 14) |
| Set | |
| 5′-agaattgatcgccaaaatcc-3′ | (SEQ ID NO: 15) |
| and | |
| 5′- ataatgcagagcctcctcgt-3′ | (SEQ ID NO: 16) |
| β-actin | |
| 5′-ccaccgatccacacagagta-3′ | (SEQ ID NO: 17) |
| and | |
| 5′-ggctcctagcaccatgaaga-3′ | (SEQ ID NO: 18) |

The following primers for human genes were used:

| XPO4 | |
|---|---|
| 5′-gggagactcttctggacagc-3′ | (SEQ ID NO: 19) |
| and | |
| 5′-gagaagctaactgggcaagg-3′ | (SEQ ID NO: 20) |
| EIF5A1 | |
| 5′-tgaagatatctgcccgtcaa-3′ | (SEQ ID NO: 21) |
| and | |
| 5′-gctgtcctggagcagtgata-3′ | (SEQ ID NO: 22) |
| EIF5A2 | |
| 5′-ggcttccagcacttacccta-3′ | (SEQ ID NO: 23) |
| and | |
| 5′-accatgctttccagttttgg-3′ | (SEQ ID NO: 24) |
| β-actin | |
| 5′-ttcaacaccccagccatgt-3′ | (SEQ ID NO: 25) |
| and | |
| 5′-gccagtggtacggccaga-3′ | (SEQ ID NO: 26) |

### Oncogenomic Studies of Human Hepatocellular Carcinoma.

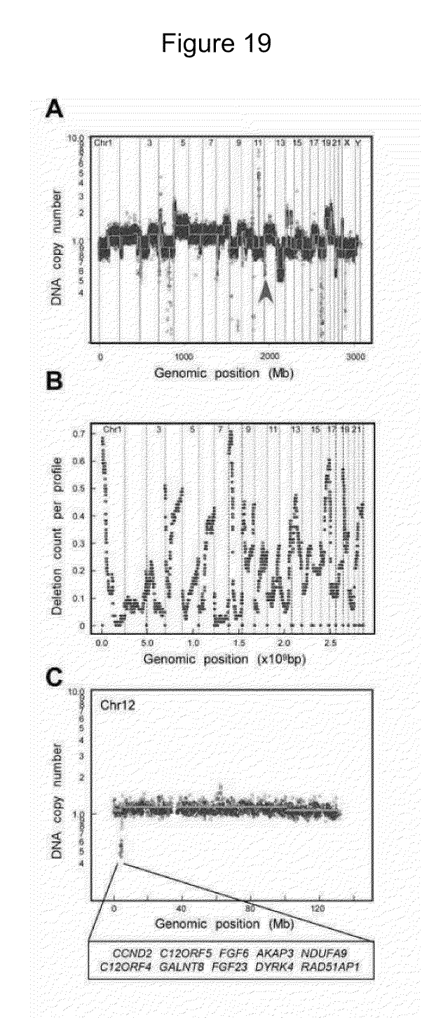

Tumor suppressor gene inactivation is often due to homozygous or hemizygous chromosomal deletions, thus producing local regions of reduced gene copy number. To identify genomic regions potentially containing tumor suppressor genes, ˜100 human hepatocellular carcinomas of different etiologies (Hepatitis B, Hepatitis C or ethyltoxic liver cirrhosis) were analyzed for DNA copy number alterations using representational oligonucleotide microarray analysis (ROMA), a high-resolution array-based CGH platform (FIG. 19A) (Lucito et al., 2003, Genome Res. 13: 2291-2305). Raw data was converted into segmented profiles using a previously described algorithm (Hicks et al., 2006, Genome Res. 16: 1465-1479), and segments that showed significant decrease from the ground state were computationally identified. To obtain a genome-wide view of chromosomal deletions, genetic events were computationally estimated so that a homozygous deletion within a heterozygous deletion would be scored as two deletion events rather than one. The results of this deletion event counting algorithm across the entire genome are shown in FIG. 19B. Among the many genomic deletions detected, only a subfraction represented focal deletions (<5 MB). These regions were likely to be enriched for tumor suppressor genes.

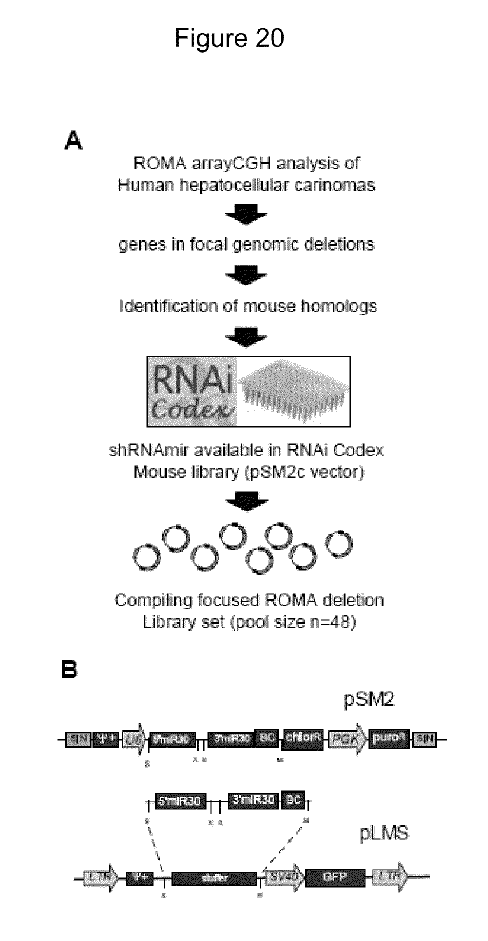

To develop an initial gene list for further studies, all of the genes embedded in recurrent focal deletions or in unique focal deletions whose gene content was also contained in broader deletions that were recurrent were identified. Based on these criteria, 58 focal deletions (<5 MB) ranging in size from 98 kb to 2.6 Mb, containing 1 to 46 genes, respectively (see, for example, FIG. 19C) were identified. These deletions contained 362 annotated genes (Table 3, see red circles in FIG. 19B), for which 301 mouse orthologs were identified using bioinformatics. All 631 of the mir30-design based shRNAs from the Cold Spring Harbor Laboratory RNAi CODEX library were then obtained. Thus, on average, each deleted gene was represented by ˜2 shRNAs targeting the corresponding mouse ortholog (see workflow in FIG. 20A).

### Reconstructing an In Vivo RNAi Screen.

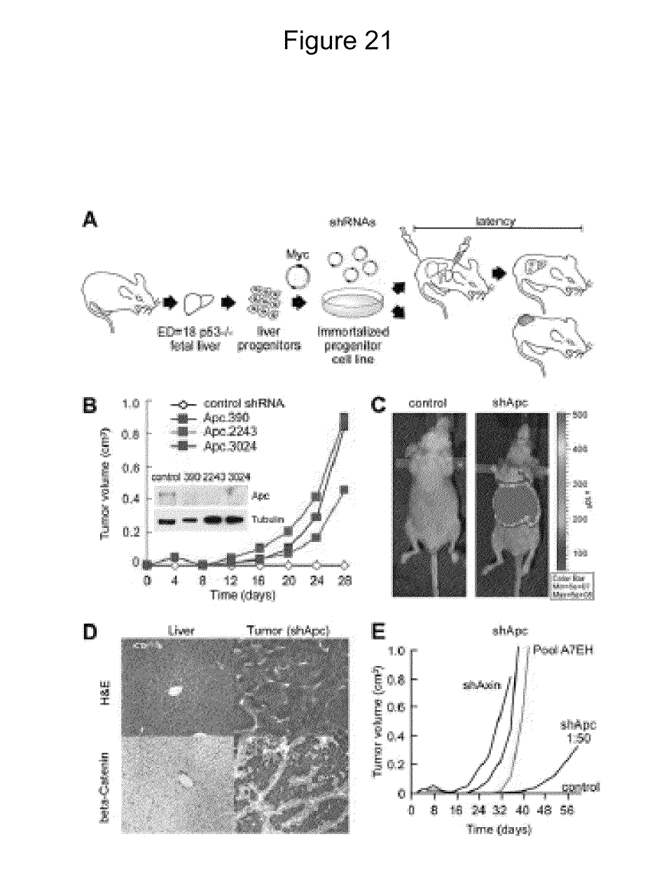

A “mosaic” mouse model was previously developed in which liver carcinomas can be rapidly produced following the isolation and genetic manipulation of liver progenitor cells and their retransplantation into recipient mice (Zender et al., 2006, Cell 125: 1253-1267). Since existing technologies did not allow RNAi to be both efficiently and stably directed to tissues in vivo, pools of shRNAs were introduced into premalignant progenitor cells and a selection for those that promoted tumor formation following transplantation was performed. Immortalized lines of embryonic hepatocytes lacking Trp53 and overexpressing Myc that are not tumorigenic in vivo, were generated previously (Zender et al., 2005, Cold Spring Harb. Symp. Quant. Biol. 70: 251-261). >40% of all human HCCs overexpress Myc and many harbor TP53 mutations or deletions (Teufel et al., 2007, World J. Gastroenterol. 13: 2271-2282). Thus, these cells can provide a “sensitized” background in which a single additional lesion can be sufficient to trigger tumorigenesis (FIG. 21A).

Screening of complex shRNA libraries requires control shRNAs to model or “reconstruct” the screen. Positive control shRNAs that target negative regulators of the WNT pathway were chosen. WNT is deregulated in a significant percentage (30-40%) of human hepatocellular carcinomas due to activating mutations in β-catenin or inactivating mutations/promoter hypermethylation of the AXIN and APC tumor suppressors (Teufel et al., 2007; Yang et al., 2003, Am. J. Pathol. 163: 1101-1107). To test whether suppression of either Axin or Apc would be tumorigenic in the system of the invention, mir30-design shRNAs targeting each gene were introduced into Trp53−/−;Myc hepatocytes, and the resulting cell populations were transplanted subcutaneously into nude mice or into the liver by intrasplenic injection (Zender et al., 2006; Zender et al., 2005). All of the shRNAs used in this study were cloned into the pLMS vector, which co-expresses the mir30-design shRNA with green fluorescent protein (GFP), and has previously been optimized for in vivo use (Dickins et al., 2005, Nat. Genet. 37: 1289-1295). Furthermore, the Myc transgene co-expressed a luciferase reporter, which enabled tumor growth to be tracked using bioluminescence imaging.

While a negative control shRNA targeting human RB1 (but not mouse) did not trigger tumor growth, the positive control shRNAs targeting Axin or Apc gave rise to tumors within 1-2 months both subcutaneously and in situ (FIG. 21A-D). The resulting tumors were classified as aggressive solid, sometimes pseudoglandular, hepatocellular carcinomas (FIG. 21D, top panel). They also displayed high levels of nuclear β-catenin by immunohistochemistry, indicating that the shRNAs were deregulating the predicted biochemical pathway (FIG. 21D, bottom panel).

In order to determine the complexity of shRNA pools that could be recovered from a complex mixture, dilutions of the Apc shRNA ranging from 1:1 to 1:100 were produced. In addition, a pool of 48 shRNAs targeting various murine genes that also contained two distinct Apc shRNAs was tested. In both situations, the diluted shRNAs produced tumors much more rapidly than the control shRNA, albeit with a delay relative to the pure Apc shRNA (FIG. 21E). Moreover, sequencing of PCR-amplified shRNAs obtained from tumors triggered by the shRNA pool revealed that the two Apc shRNAs were highly enriched during tumor expansion, comprising the vast majority of shRNAs present in the resulting tumor cells.

Thus, pools of 48 shRNAs can be readily screened to identify those with tumor promoting activities similar to the Apc shRNAs. Although tumor growth was enhanced by 1:100 dilutions of the Apc shRNA, screening of low complexity pools is feasible and can maximize the chance of identifying weaker shRNAs that might otherwise be outcompeted by stronger ones in more complex pools.

Promotion of Tumorigenesis In Vivo by shRNA Pools Targeting Genes Deleted in Liver Cancer.

To screen the shRNA library targeting deletion-associated genes, individual CODEX clones were pooled randomly into pools of 48 and transferred in bulk into pLMS (see FIG. 20B). Selected shRNA library pools were subjected to DNA sequencing to confirm that clone representation was maintained. As controls, 10 pools of randomly selected shRNAs from the mouse genome-wide CODEX RNAi library (i.e. not selected based on genomic location) were also produced. Each pool was introduced into Trp53−/−;Myc hepatocytes at a low multiplicity of infection. The resulting cell populations were transplanted subcutaneously into both flanks of four immunocompromised mice, which were subsequently monitored for tumor development in comparison to the parental cells transformed with the negative shRNA control.

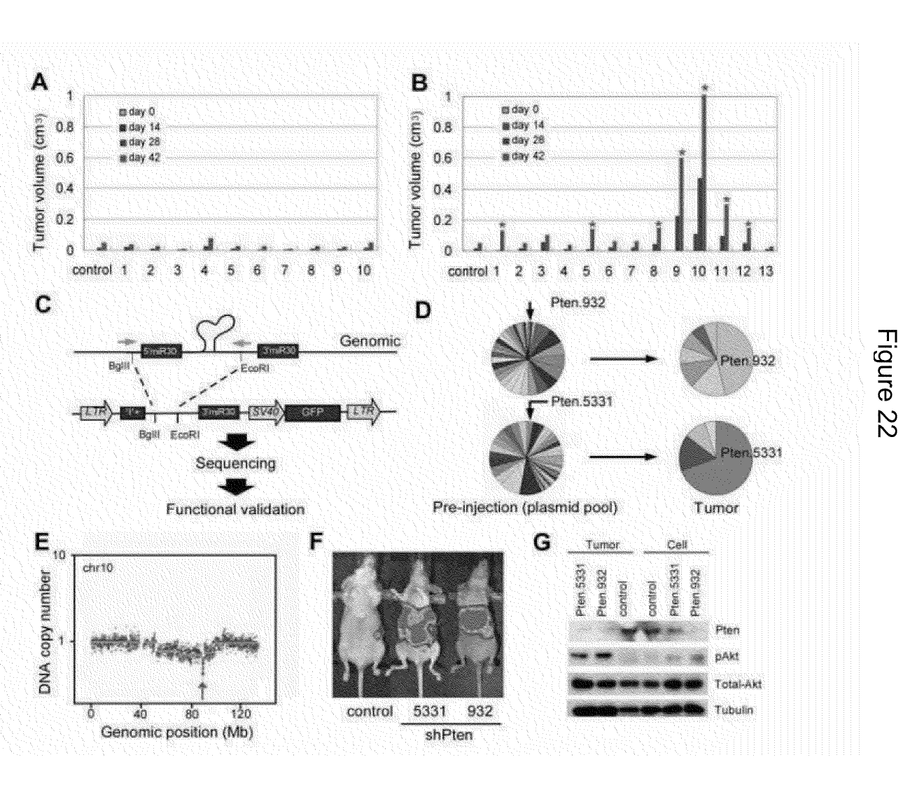

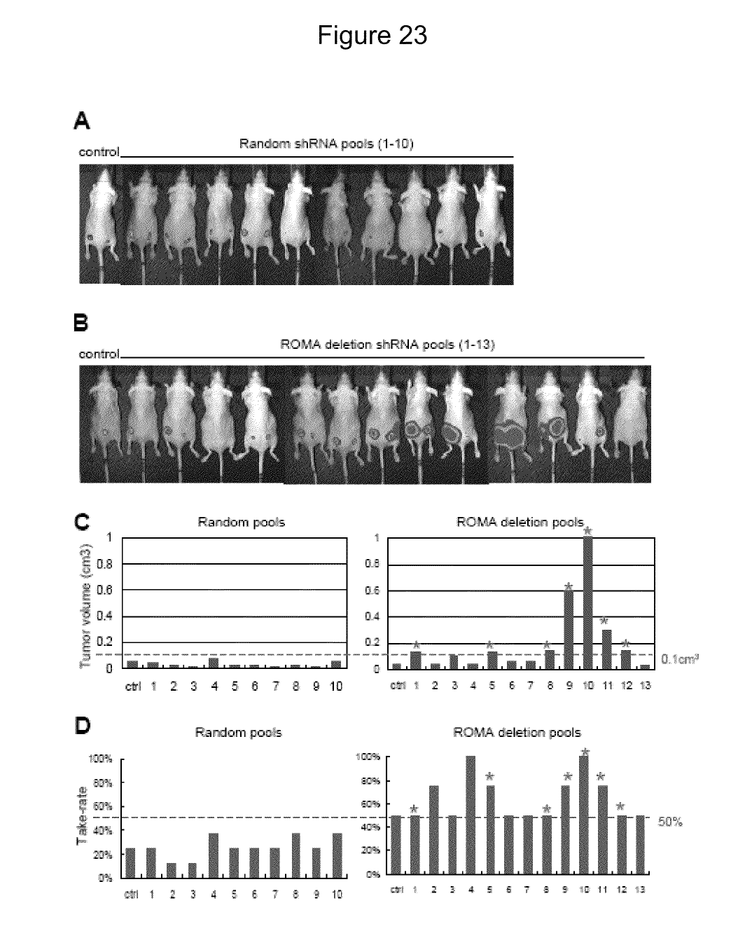

The results of these experiments were striking: while mice injected with cells containing randomly produced shRNA pools barely developed tumors over background (FIG. 22A and FIG. 23A), many mice transplanted with cells containing the deletion-based shRNA pools developed tumors at the injection sites, some within 3-4 weeks (FIG. 22B and FIG. 23B). Many tumors appeared multifocal and all were GFP positive, indicating that the tumor cells contained at least one shRNA. These observations validate the enrichment strategy described herein and show that deletion-focused shRNA libraries are enriched for tumor promoting shRNAs

Identification of Candidate Tumor-Promoting shRNAs.

To identify candidate shRNAs present in tumors, genomic DNA was isolated from GFP-positive tumor nodules, the integrated shRNAs were PCR amplified and then cloned into a recipient vector that could also be used for subsequent validation (FIG. 22C). Of note, as cut-off criteria for further studies, shRNAs from those tumors that were relatively large, and derived from pools that produced tumors at high frequency (average tumor volume <0.1 cm³ and ≧50% take rate), were isolated. None of the random shRNA pools fit these two criteria (FIGS. 23C and 23D). The plasmids were sequenced to determine the representation of particular shRNAs (96 sequence reads/tumor, >3 tumors/pool). In most cases, more than one shRNA was identified from each tumor nodule, consistent with the possibility the tumors were multiclonal.

Interestingly, two independent shRNAs targeting Pten were identified as highly enriched in tumors produced from cells transformed with two different shRNA pools (FIG. 22D). By comparing the relative representation of each Pten shRNA to that in the initial pool, it was noted that Pten.932 (HP_{—}524) was enriched from 3% to 41% during tumor expansion (FIG. 22D, upper panel), whereas Pten.5331 (HP_{—}465354) went from 1% to 67% of the total sequence reads (FIG. 22D, lower panel). Immunoblotting revealed that both shRNAs suppressed Pten protein expression and increased phosho-Akt levels (FIG. 22G), indicating that they were biologically active. Interestingly, while shRNA PTEN.932 produced more efficient PTEN knockdown (and higher p-Akt levels) than shRNA PTEN.5331 in the preinjected cell population, the resulting tumors showed similar PTEN knockdown and p-Akt levels. These results show that cells with optimal knockdown are selected during tumor expansion (FIG. 22G).

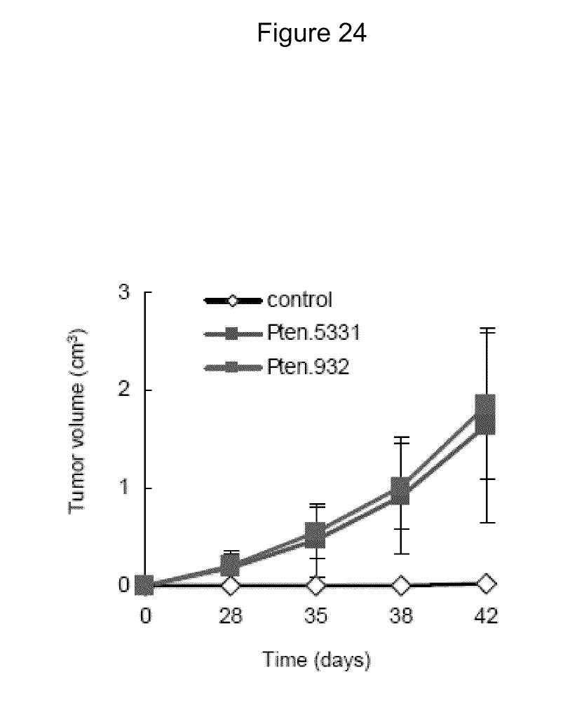

PTEN is a bona fide tumor suppressor gene that is lost or mutated in many tumor types (Tokunaga et al., 2008, Curr. Cancer Drug Targets 8: 27-36) and whose deletion can trigger hepatocarcinogenesis in mice (Horie et al., 2004, J. Clin. Invest. 113: 1774-1783). Consistent with this, several HCCs we identified with either focal or broad chromosome 10 deletions encompassing PTEN (FIG. 22E). To confirm that suppression of Pten is oncogenic in the model system described herein, we re-tested the recovered Pten shRNAs by introducing them into Trp53−/−;Myc hepatocytes and testing their ability to form tumors following subcutaneous or intrasplenic injection. In both contexts, Pten knockdown rapidly triggered tumor growth (FIG. 22F and FIG. 24), thus validating the screening strategy described herein and providing a blueprint for testing shRNAs targeting additional, less characterized, genes.

### Novel Tumor Suppressors Identified and Validated In Vivo.

shRNA representation was systematically analyzed in all 31 tumors (derived from seven shRNA pools) that showed an accelerated tumor growth (average tumor volume ≧0.1 cm³ and ≧50% take rate) (FIG. 22B; Table 2). From a total of 2307 sequence reads, 36 shRNAs were identified that were enriched at least 2.5 fold over the predicted representation in the initial plasmid pool (˜2% of total). For example, the two validated Pten shRNAs ranked the 5^{th} and 16^{th}, respectively, among the most enriched shRNAs (Table 4). Besides Pten, 16 shRNAs targeting 14 different genes were selected for further validation (FIG. 25A-B, Table 4). These included all genes for which shRNAs were most abundant in at least two tumors (Xpo4, Armcx2, Nrsn2, Zbbx), all genes for which we found enrichment of two independent shRNAs (Fgf6, Set, Fstl5) and a group of seven genes with single scoring shRNAs that were highly enriched (Wdr49, Wdr47, Armcx1, Gjd4, Glo1, Ddx20, Btbd9). Interestingly, although SET (histone chaperone/protein phosphatase inhibitor) was originally identified as part of an oncogenic fusion in acute myeloid leukemia (von Lindern et al., 1992b, Baillieres Clin. Haematol. 5: 857-879), none of the other genes had previously been linked to cancer. Nevertheless, their presence in focal genomic deletions found in human liver tumors, as described herein, suggests that their inactivation may contribute to tumorigenesis (FIG. 25B).

A total of 16 shRNAs targeting 14 different genes were functionally validated using the same experimental setup employed in the initial screen. A validated Pten shRNA (Pten.5331) was used as a positive control in each experiment. As shown in FIG. 25C, many other candidate shRNAs triggered tumor growth above background, with those targeting Xpo4 (nuclear export protein), Ddx20 (GEMIN3, RNA helicase), Gjd4 (CX40.1, putative gap junction protein), Fstl5 (Follistatin-like 5) and Nrsn2 (Neurensin 2) showing the most prominent acceleration of tumor growth (FIG. 25C). Since control shRNA never accelerated tumorigenesis, it is unlikely that insertional mutagenesis was solely responsible for the biological effects of the candidate shRNAs. On the contrary, the data suggests that suppression of the targeted gene was essential.

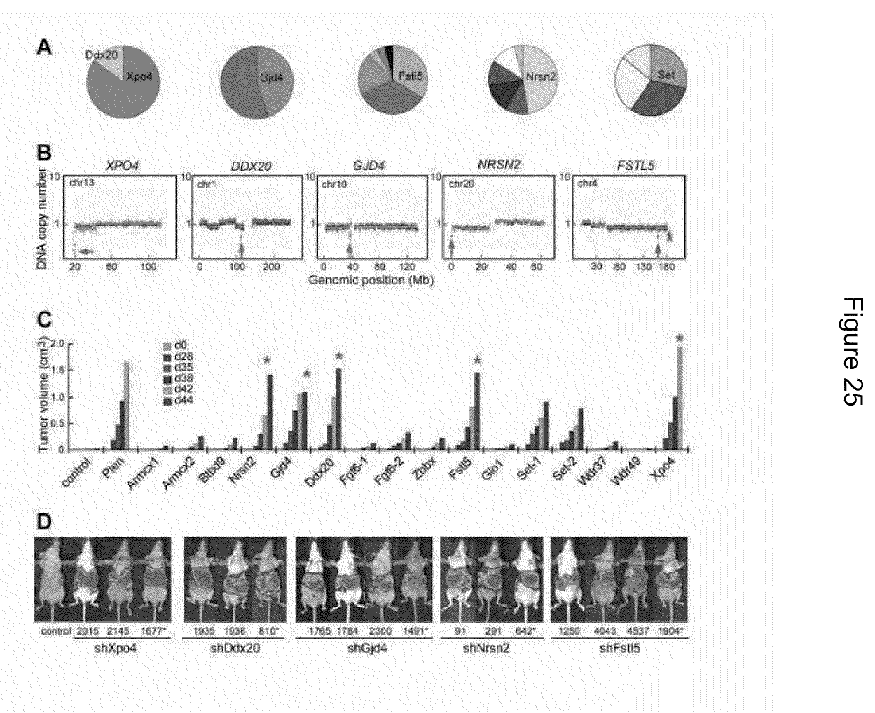

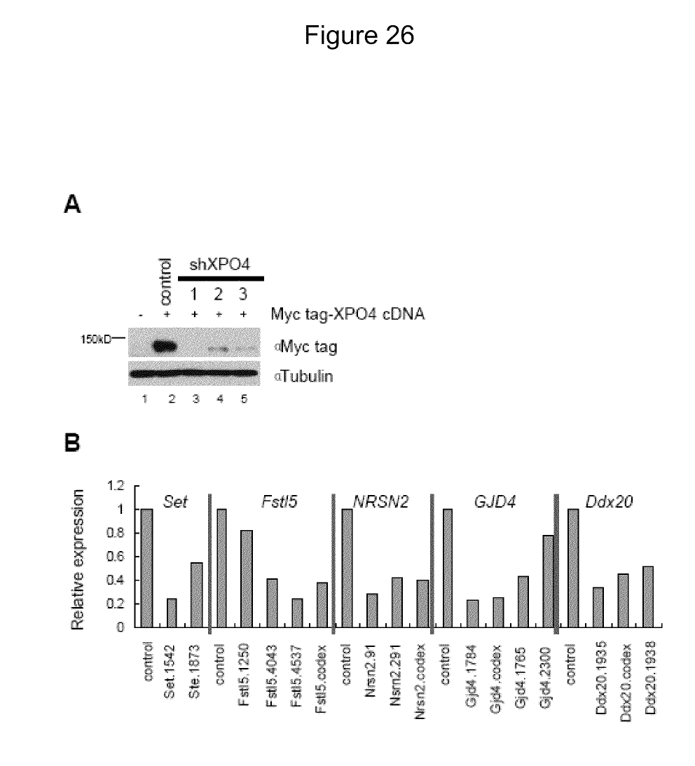

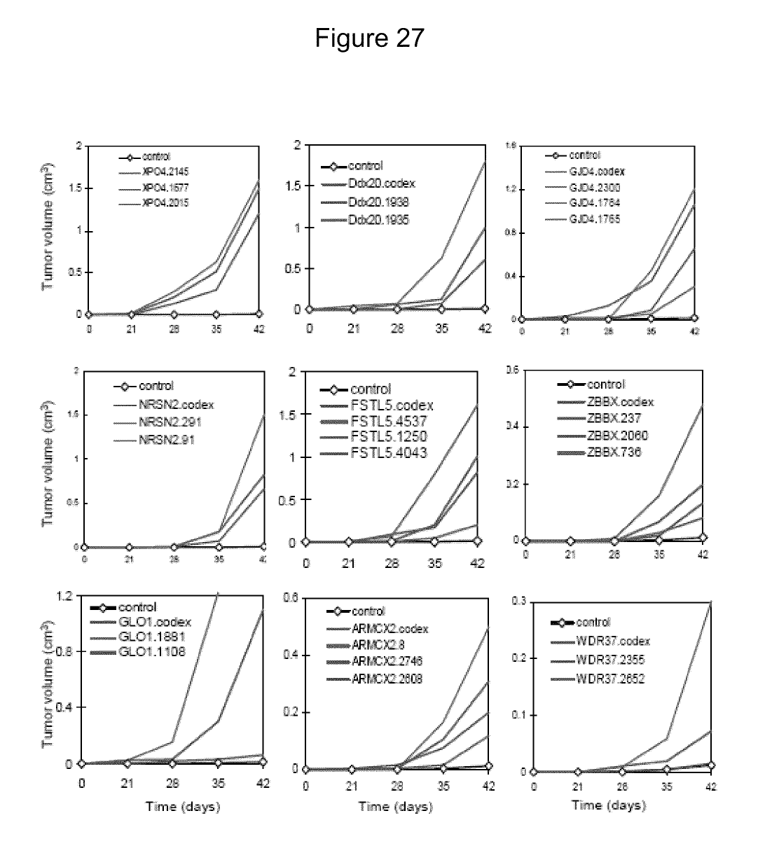

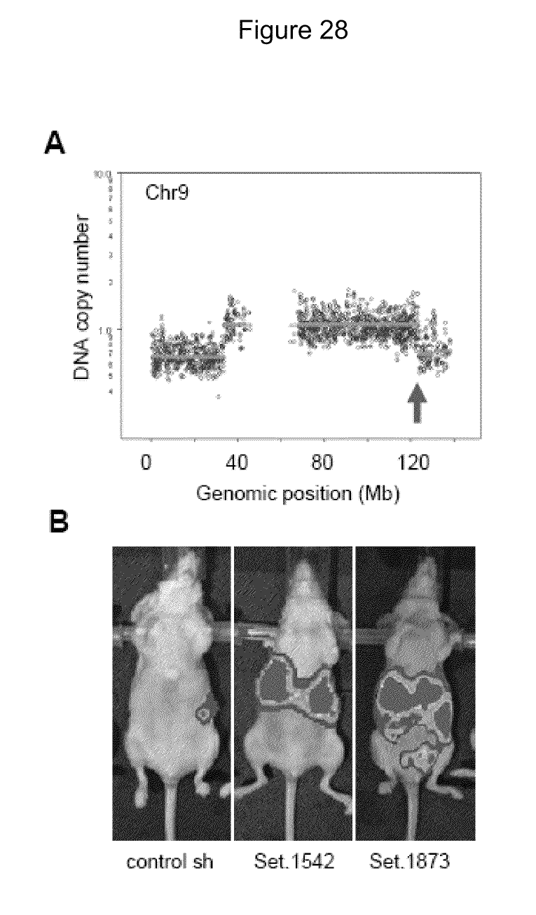

To rule out the possibility that off-target effects of RNAi might be responsible for the tumor-promoting effects of individual shRNAs, at least two additional shRNAs against each candidate gene were generated and tested, together with those identified in the screen, for their ability to promote hepatocarcinoma development in situ. Trp53−/−;Myc liver progenitor cells were transformed with shRNA encoding retroviruses, and knockdown of the predicted target gene was confirmed by either immunoblotting or quantitative RT-Q-PCR (FIG. 26). The resulting cell populations were transplanted into the livers of recipient mice by intrasplenic injection, and the animals were monitored for tumor formation using bioluminescence imaging. In all cases, at least three independent shRNAs targeting each candidate promoted tumors by 60 days post-injection, a time when there was little if any detectable luciferase signal in controls (FIG. 25D, see also FIG. 27 and FIG. 28 for results from subcutaneous injections). Similar results were obtained using independent populations of Trp53−/−;Myc liver progenitor cells (FIG. 29). Therefore, the genes identified using the in vivo RNAi screen described herein are bona fide tumor suppressors in mice.

### Inactivation of XPO4 Deregulates SMAD3 and EIF5A Signaling.

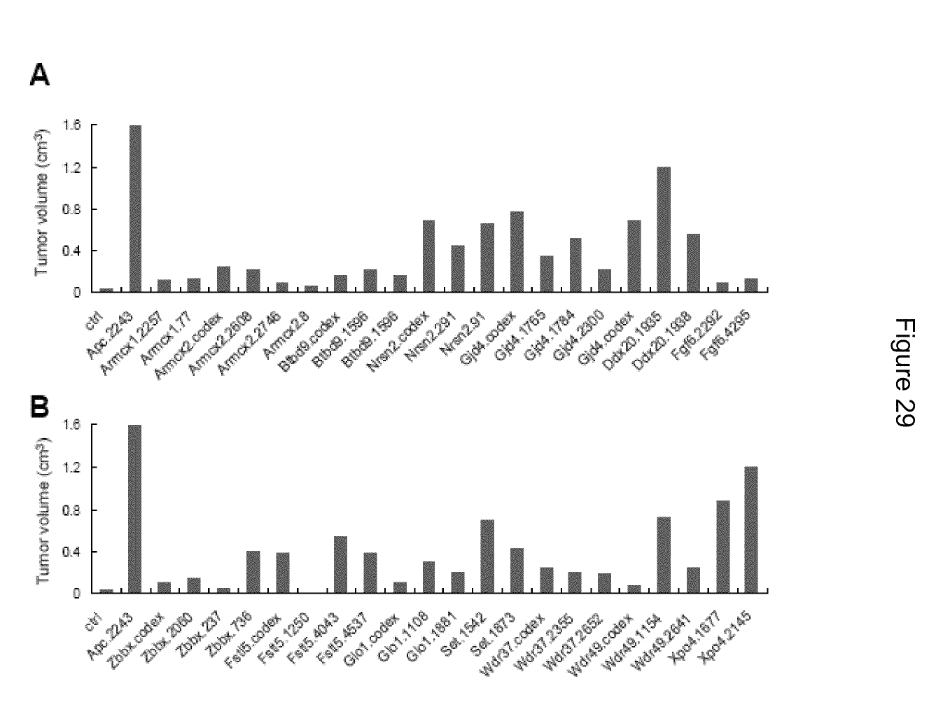

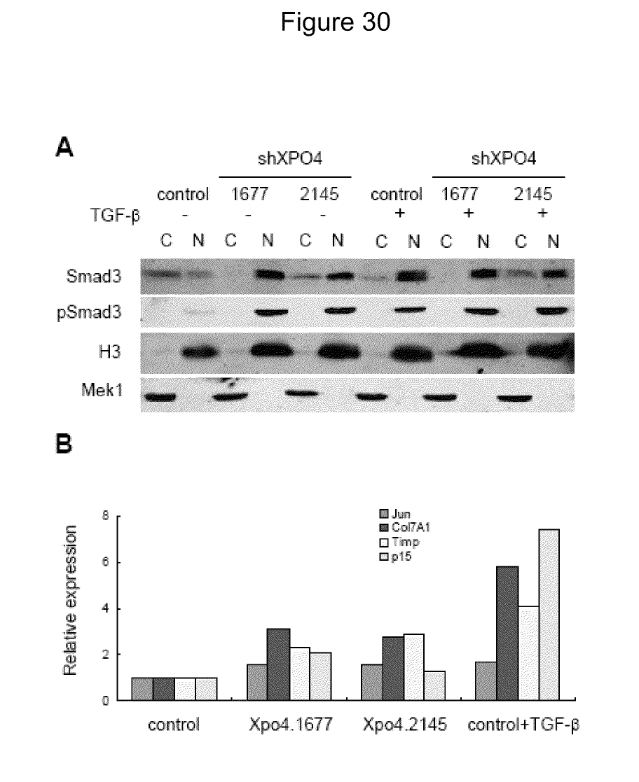

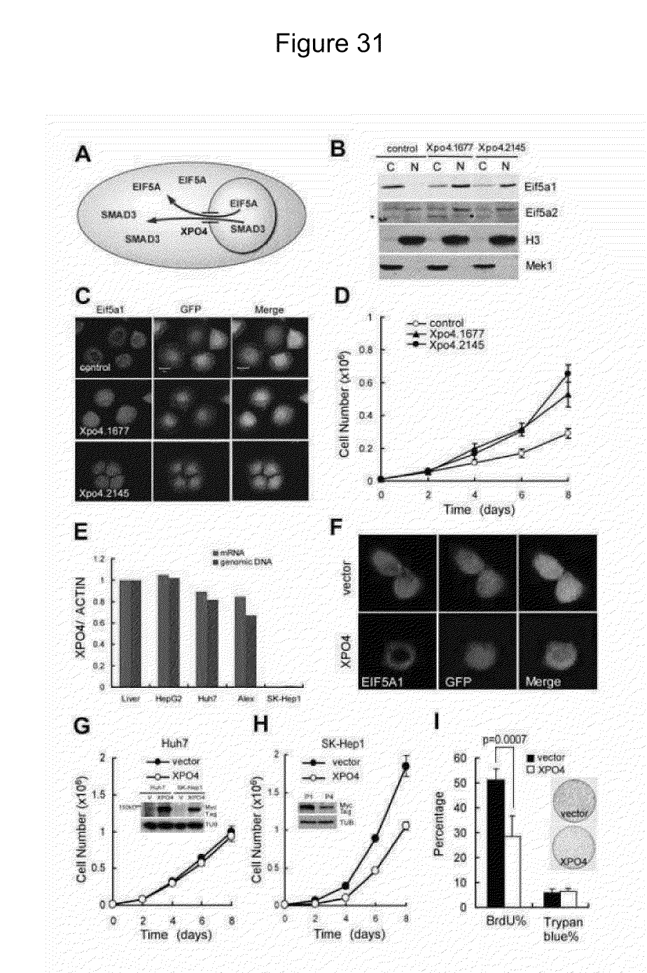

The shRNA that was most enriched in the screen described herein targeted Xpo4, and subsequent validation experiments indicated that Xpo4 shRNAs were the most potent at triggering tumor growth (FIG. 25C-D). Exportin 4 (encoded by Xpo4) belongs to the importin-β family of nuclear transporters and has two known substrates—SMAD3 and EIF5A1 (FIG. 31A). SMAD3 is an effector of the TGF-β signaling cascade and can have pro- or anti-oncogenic effects, but its activation is associated with hepatocellular carcinoma progression (Teufel et al., 2007, World J. Gastroenterol. 13: 2271-2282). In response to TGF-β, SMAD3 and other SMADs become phosphorylated and shuttle to the nucleus, where they form complexes with coactivators to transactivate TGF-β target genes (Massague, 2000, Nat. Rev. Mol. Cell Biol. 1: 169-178). Murine hepatoma cells expressing Xpo4 shRNAs showed an increase in nuclear total and phospho-Smad3, as assessed in fractionated cell lysates (FIG. 30A). Cells expressing Xpo4 shRNAs consistently showed an increase in the TGF-β target genes Jun, Col7a1, Timp1 and p15, compared to control shRNA (FIG. 30B). XPO4 may influence tumorigenesis by modulating SMAD3 function.

Prior to the present invention, little was known about the biological role(s) of the XPO4 substrate EIF5A. EIF5A was identified as a eukaryotic translation initiation factor, and is conserved throughout eukaryotes. In mammals, it is encoded by two highly related genes, EIF5A1 and EIF5A2. Although in vitro studies suggest that EIF5A stimulates the formation of the first peptide bond during protein synthesis (Kemper et al., 1976, J. Biol. Chem. 251: 5551-5557; Benne et al., 1978, J. Biol. Chem. 253: 3078-3087), EIF5A may also influence nucleocytoplasmatic transport of mRNA and/or mRNA stability (reviewed in Caraglia et al., 2001, Amino Acids 20: 91-104). Nevertheless, as was observed for Smad3, knockdown of Xpo4 in murine hepatoma cells led to nuclear accumulation of Eif5a1 and Eif5a2 (FIG. 31B-C). Thus, inactivation of Xpo4 alters Eif5a signaling in liver cells.

Reintroduction of XPO4 Specifically Suppresses Tumors with XPO4 Deletion.

Interestingly, Xpo4 shRNAs enhanced the proliferation of premalignant cells in vitro, suggesting that one role of Xpo4 is to restrain cell proliferation (FIG. 31D). The consequences of enforced Xpo4 expression in human HCC cell lines expressing and lacking XPO4 was tested (see FIG. 31E for a cell line, SK-Hep1, that contains a homozygous deletion of XPO4 and thus does not express the gene). SK-Hep1 cells (XPO4-negative) and Huh7 cells (XPO4-positive) were infected with retroviruses encoding a myc-tagged XPO4 cDNA and the resulting cell populations were examined for XPO4 expression (FIG. 31G inlay), EIF5A localization (FIG. 31F), and proliferation (FIG. 31G-I). XPO4 was expressed in both cell types, albeit at slightly lower levels in SK-Hep1 cells (FIG. 31G inlays). Nevertheless, enforced expression of XPO4 had no impact on the proliferation of XPO4-positive cells (Huh7, FIG. 31G), but substantially slowed down the proliferation of XPO4-negative cells (SK-Hep1, FIG. 31H), primarily by attenuating cell cycle progression (BrdU incorporation) and not by cell death (FIG. 31I). Interestingly, the expression level of XPO4 in SK-Hep1 cells substantially declined during passaging, suggesting a strong selective pressure against XPO4 in these cells (FIG. 31H inlay). These results not only suggest that loss of XPO4 function is relevant to tumor maintenance, but also extend th efindings to a human system, and suggest that XPO4 can attenuate cell cycle progression.

### XPO4 and EIF5A Define a Novel Oncogenic Signaling Circuit.

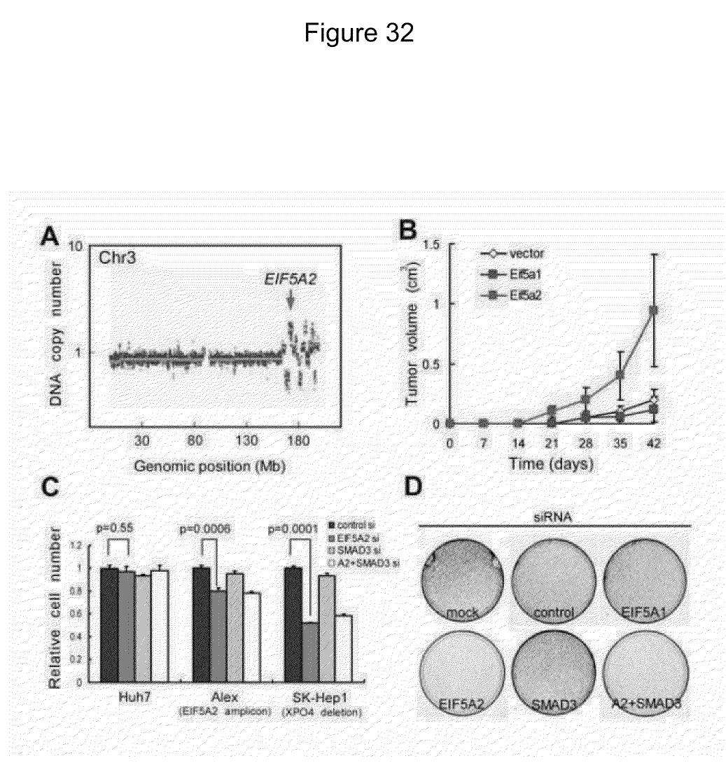

Both EIF5A proteins are overexpressed in some human tumors (Wang et al., 1997, Arch. Toxicol. 71: 450-454; Gopalkrishnan et al., 1999, Int. J. Biochem. Cell Biol. 31: 151-162) and, interestingly, the EIF5A2 gene is often co-amplified with PIK3CA (encoding a catalytic subunit of PI3 kinase) on chromosome 3q26 (Guan et al., 2004, Cancer Res. 64: 4197-4200; Guan et al., 2001, Cancer Res. 61: 3806-3809). In human HCCs 22 tumors were identified with amplifications on chromosome 3 that encompass the EIF5A2 gene (FIG. 32A), and at least three that excluded PIK3CA. Therefore, to determine whether overexpression of EIF5A2, like XPO4 loss, would promote hepatocellular carcinoma development, an Eif5a2 cDNA was retrovirally transformed into Trp53−/−;Myc hepatocytes and injected the cells subcutaneously into nude mice. Remarkably, Eif5a2, but not Eif5a1, efficiently triggered the growth of tumors (FIG. 32B), which showed histopathological features of human hepatocellular carcinoma.

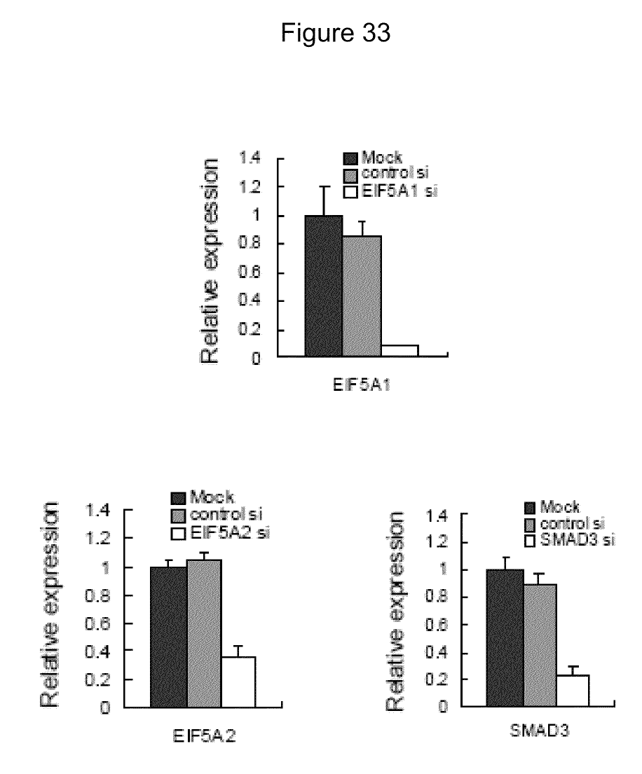

To examine the requirement for XPO4 substrates in maintaining the proliferation of human tumor cells, siRNAs targeting EIF5A2 and SMAD3 were transfected into human tumor cells harboring XPO4 deletion (SK-Hep1), EIF5A2 amplification (Alex), or neither genomic alteration (Huh7), and examined their impact on cell proliferation. Whereas each set of siRNAs efficiently suppressed the respective target (FIG. 33), only siRNAs targeting EIF5A2 inhibited proliferation in short term (MTT) and long term (colony formation) assays (FIG. 32C-D). Moreover, the anti-proliferative effects of these siRNAs were largely restricted to cells that ampflied EIF5A2 or, more prominently, deleted XPO4 (FIG. 32C). These data implicate EIF5A2 as a key mediator for the oncogenic effects of XPO4 loss, though it is possible that modulation of SMAD3 activity may also play a role, perhaps by enhancing tumor invasion and metastasis in vivo. Nevertheless, these results indicate that EIF5A2 is indeed a key downstream effector of XPO4 in tumor suppression and that, together, these proteins encode a new signaling module relevant to human oncogenesis.

Relevance to Other Tumor Types of Genes Identified from HCC Screens.

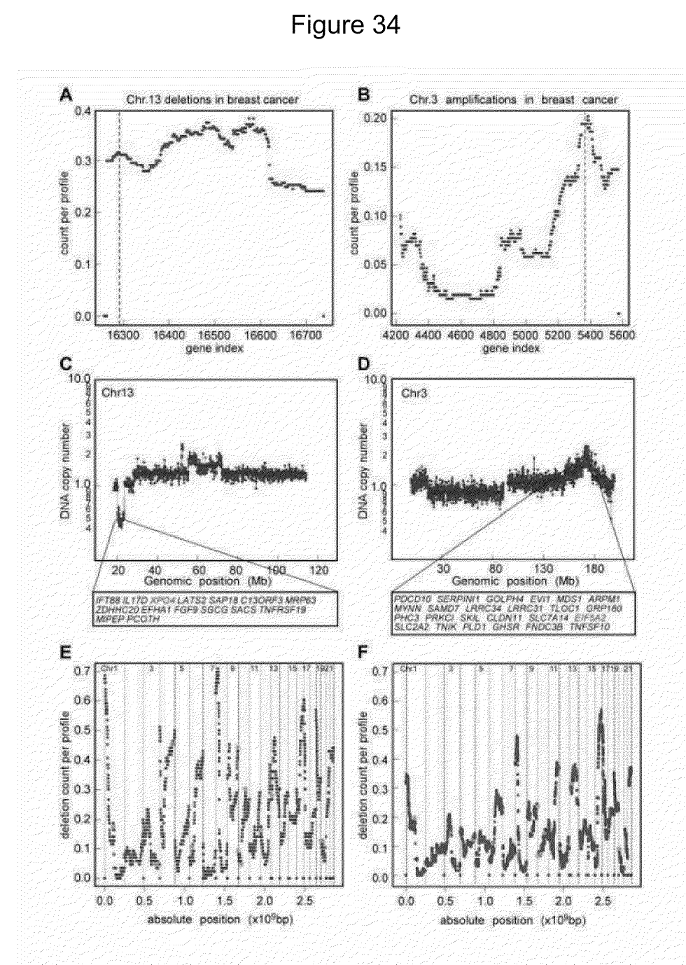

Although our screen focused on hepatocellular carcinoma, the tumor suppressors identified herein can be relevant to other tumor types. Indeed, mutations in APC and AXIN (here used as positive controls) are observed in colon carcinomas, medulloblastomas and other cancers (Segditsas et al., 2006, Oncogene 25: 7531-7537; Salahshor et al., 2005, J. Clin. Pathol. 58: 225-236), and PTEN (identified in our screen) loss occurs in brain, lung, colon, breast, pancreatic and prostate cancers (Chow et al., 2006, Cancer Lett. 241: 184-196). As a first step in expanding the analysis, a database containing copy number analyses of over 257 breast cancers of various pathologies, tumor size, grade, node involvement, and hormone receptor status (Hicks et al., 2006, Genome Res. 16: 1465-1479) was surveyed. Gene deletion frequencies were produced from comparative genomic hybridization as described for the HCC data (FIG. 34A); in addition, gene amplification profiles were produced in a parallel manner (FIG. 34B). Remarkably, XPO4 was found to be located at a local deletion epicenter on chromosome 13, which occurs in over 30% of tumors (FIG. 34A, see example in FIG. 34C) and is associated with poor survival in a large cohort of breast cancer patients (Table 6, p=0.038). Interestingly this region often also includes the LATS2 gene, which is a tumor suppressor in Drosophila (Yabuta et al., 2000, Genomics 63: 263-270). Of note, shRNAs targeting LATS2 were not included in the screens described herein, as it was excluded from the focal deletions found in liver cancer.

Similarly, the exportin 4 substrate EIF5A2 was found to be near the epicenter of amplifications located on human chromosome 3q26 (FIG. 34B, see example in FIG. 34D), which is frequently observed in breast cancer. These amplifications were often focal (see FIG. 34D), but frequently contained many additional genes. Although the PIK3CA gene has previously been proposed as a candidate oncogene on 3q26, at least three breast cancers containing the 3q26 amplicon did not amplify PIK3CA. Together with the functional analysis described herein, these observations suggest that EIF5A2 can be a driving oncogene (see also Guan et al., 2004, Cancer Res. 61: 3806-3809). Of note, other validated tumor suppressors identified in this screen (FIG. 34E) were also located in focal deletions in human breast cancer (FIG. 34F), including PTEN, FGF6, NRSN2 and GLO1/BTBD9 (Table 5). This study thus supports the utility of RNAi screens in a liver carcinoma model to identify tumor suppressors relevant to diverse tumor types.

The strategy outlined herein describes a new approach to cancer gene discovery. Many of the current efforts to catalog the genes linked to human cancer rely on genomic approaches alone; however, a limitation of such strategies is that they are expensive and yield candidates based primarily on statistical criteria. Virtually all candidates must be functionally validated in various in vitro or in vivo models, which is slow and likewise expensive. By incorporating the screening approach described herein, it is possible to rapidly filter genomic information on those genes that impact cancer development in vivo, and thus focus follow-up studies on those that might be most clinically useful. Of note, the experiments described in this Example utilize a mouse model of hepatocellular carcinoma and focuses on focal deletions. However, this relatively high throughput approach can be extended to other mouse models, and can include shRNAs targeting genes in larger deletions, genes that are methylated or mutated in human cancers, or even genes enriched in insertional mutagenesis screens in mice. Moreover, by exploiting the emerging libraries of full length cDNAs, it is possible to functionally screen for oncogenes involved in genomic amplifications. Such integrative approaches provide a cost-effective strategy for the functional annotation of the cancer genome.

The experiments described herein provide an RNAi screen for genes that affect complex phenotypes in mice. To identify new tumor suppressor genes relevant to hepatocellular carcinoma, information obtained from an oncogenomic analysis of ˜100 human tumors was combined with in vivo studies to screen for shRNAs that promote hepatocellular carcinoma in a mouse model. This approach was based on the premise that loci deleted in human cancers are enriched for tumor suppressor genes and, indeed, the data presented herein shows that pools of shRNA corresponding to deleted genes in human hepatocellular carcinoma frequently “score” at promoting tumors, whereas pools containing random shRNAs do not. By identifying and retesting the enriched shRNAs, large number of candidate tumor suppressors were identified, 14 whose suppression reproducibly promoted tumorigenesis in mice. Given that some of the shRNAs in the library may not suppress their cognate target gene sufficiently to produce phenotypes in vivo, this approach is remarkably efficient.

The fact that random pools of shRNAs did not promote tumorigenesis in the experiments described herein implies that many of the genomic deletions observed in human tumors produce a selective advantage and are not “passenger” lesions coincidentally linked to oncogenesis. This notion also explains why some cancer-associated deletions occur repeatedly in different tumor types. Still, some loci may be particularly susceptible to deletion, for example at fragile sites, and such deletions may be recurrent without conferring a selective advantage to the tumor cell (Durkin et al., 2008, Proc. Natl. Acad. Sci. U.S.A. 105: 246-251). While it is possible that deletions that did not score in our assay may arise from genomic instability, fragile site deletions are not present or extremely rare in primary liver tumors, unlike in other tumor types such as lung cancer (Zochbauer-Muller et al., 2000, Clin. Lung Cancer 2: 141-145). In any case, the present data documents the value of using genomic deletions as a filter in identifying functionally relevant tumor suppressor genes.

Surprisingly, three of the ten focal deletions that scored in the system contain multiple genes whose knockdown accelerates tumorigenesis in mice. Moreover, XPO4 and EIF5A2 are adjacent to a strong candidate tumor suppressor (LATS2) and oncogene (PIK3CA), respectively. That some genomic regions can contain closely linked cancer genes is not unprecedented; for example, the 11q22 amplicon found in human liver cancer and other tumor types contains two genes—BIRC2 and YAP—whose overexpression can cooperate in tumorigenesis (Zender et al., 2006, Cell 125: 1253-1267), and the MYC amplicon on chromosome 8 often co-amplifies a non-coding RNA that contributes to maintenance of cell viability (Guan et al., 2007, Clin. Cancer Res. 13: 5745-5755). Conversely, focal deletions on chromosomal region 9p21 often simultaneously co-delete the INK4a (CDKN2A, isoforms 1 & 3), INK4b (CDK2NB), and ARF (CDKN2A, isoform 4) tumor suppressor genes, which can act in combination to suppress tumorigenesis (Krimpenfort et al., 2007, Nature 448: 943-946). The biological rationale for such genomic organization is not understood, but it is possible that these genes are co-regulated at the level of higher order chromatin organization for some purpose during normal growth control or development. Interestingly, shRNAs targeting the linked tumor suppressors identified here were not as potent as others identified in the screen.

The tumor suppressor genes validated herein target a remarkable array of biological activities. Genes such as AXIN (which targets the β-catenin pathway and was used as positive control) as well as PTEN (a modulator of the PI3-kinase pathway and identified from the screen) have been implicated in liver cancer based on their somatic alteration in human tumors. The present data therefore solidifies the importance of these genes in liver cancer and develops tractable animal models that may be useful for future functional or preclinical studies. In addition, the SET gene, which encodes a histone chaperone and potential protein phosphatase inhibitor, was initially identified as part of a translocation in a human AML patient (von Lindern et al., 1992b, Baillieres Clin. Haematol. 5: 857-879). Although this protein clearly has oncogenic activity in the context of the fusion protein (von Lindern et al., 1992a, Genes Chromosomes Canc. 5: 227-234; von Lindern et al., 1992b), the present study suggests that in solid tumors, the native protein is a tumor suppressor.

By contrast, the vast majority of genes identified herein had not previously been linked to cancer. For example, FGF (FGF6), an RNA helicase (DDX20/GEMIN3), a metabolic enzyme (GLO1), and a gap junction protein, GJD4 (CX40.1), were identified which all apparently act as tumor suppressors in vivo.

The study described herein utilized Trp53 loss and Myc as cooperating genetic lesions owing to their common occurrence in human tumors. It is possible that other tumor suppressors would be identified screening in different genetic contexts. Indeed, some of the shRNAs identified enhance proliferation in the presence of oncogenic Ras, whereas others do not. In any case, these results greatly expand the understanding of the genetics of human hepatocelluar carcinoma and point to the potential of non-biased in vivo RNAi screens to identify potentially new and understudied areas of cancer biology. The present teachings can be used to identify novel tumor suppressors in genetic contexts other than the Trp53−/−;Myc background exemplified herein.

The top scoring candidate gene, exportin 4, belongs to the importing superfamily of nuclear transporters, and mediates the nuclear export of SMAD3, EIF5A1 and EIF5A2. Mutations that compromise XPO4 function likely act to promote tumorigenesis by leading to the nuclear accumulation of key substrates. Indeed, deregulated signaling through the WNT and AKT pathways is thought to influence tumorigenesis by enhancing, or reducing, nuclear accumulation of β-catenin and FOXO, respectively. However, other than rare AML-associated fusion proteins targeting the nuclear pore machinery (reviewed in Kau et al., 2004, Nat. Rev. Cancer 4: 106-117), most previously identified mutations affect transport-associated signaling pathways, and not the nuclear transport machinery itself That XPO4 deletions are relatively common suggest that this may be an important mechanism of oncogenesis.

Although XPO4 was not previously linked to cancer, its substrates SMAD3 and EIF5A show activities consistent with its ability to modulate tumorigenesis. Namely, SMAD3 is a modulator of the TGF-β-pathway, which can be anti-oncogenic or pro-oncogenic depending on context. The extent to which SMAD3 mislocalization contributes to the oncogenic effects of XPO4 loss remains to be determined; however, suppression of XPO4 increases the expression of TGF-β target genes (FIG. 30B), which has been linked to invasion and metastasis in late-stage liver cancer (Teufel et al., 2007, World J. Gastroenterol. 13: 2271-2282). Similarly, EIF5A2 overexpression occurs in many tumor types [(Lamberti et al., 2004, Amino Acids 26: 443-448) and references therein]. EIF5A was initially purified from rabbit reticulocytes as a translation initiation factor (Kemper et al., 1976, J. Biol. Chem. 251: 5551-5557), though recent studies suggest that EIF5A may have other activities (Caraglia et al., 2001, Amino Acids 20: 91-104). Here loss of XPO4 enhances proliferation in a manner that depends on e1F5A2, which is itself oncogenic in mice. Furthermore, re-expression of XPO4 specifically represses proliferation in XPO4-deficient tumor cells, suggesting these cells have become dependent on its loss. These results indicate that XPO4 is a negative regulator of EIF5A2 that acts, presumably in the nucleus, to inhibit cellular proliferation. Although a precise biochemical mechanism remains to be determined, the genetic and biological data suggest that the XPO4-EIF5A2 signaling circuit is relevant to human liver cancer and perhaps other tumor types.

Some of the genes identified herein point toward new strategies for cancer therapy. For example, several new tumor suppressors (here, FGF6 and FSTL5) encode secreted proteins whose systemic administration might restore tumor suppressor function and serve as new biological anti-cancer therapies. Similarly, XPO4 inactivation leads to hyperactivation of SMAD3/TGF-β signaling and, in principle, may sensitize cells to SMAD3 inhibitors, now in clinical trials (Lahn et al., 2005, Expert. Opin. Investig. Drugs 14: 629-643). Furthermore, the studies presented herein suggest that inhibition of EIF5A2 activity can have anti-tumor effects in XPO4-deficient tumors. Of note, EIF5A1 and EIF5A2 are the only eukaryotic proteins containing the polyamine derived amino acid hypusine [N^{ε}-94-amino-2-hydroxybutyl)lysine], which is required for their activity (Park et al., 1993, Biofactors 4: 95-104) and whose biogenesis can be inhibited by small molecule drugs that have anti-proliferative effects in vitro (Park et al., 1994, J. Biol. Chem. 269: 27827-27832; Clement et al., 2002, Int. J. Cancer 100: 491-498). Since XPO4 loss is associated with poor survival in breast cancer patients (Table 6), agents that target this pathway may be clinically important.

TABLE 1
shRNAs Enriched to Greater than 5% of
the Total Sequencing Reads in Each Pool

| | | |
|---|---|---|
| | | shRNA specific |
| | Mouse (HUMAN) gene | reads/total reads |
| Hairpin ID | name | of pool |

| | | | |
|---|---|---|---|
| HP_76455 | Xpo4 (XPO4) | 351/560 | (62.6%) |
| HP_253140 | Fgf6 (FGF6) | 175/342 | (51.1%) |
| HP_345037 | Wdr49 (WDR49) | 137/342 | (40%) |
| HP_89078 | Armcx2 (ARMCX2) | 88/290 | (30.3%) |
| HP_465354 | Pten (PTEN) | 66/231 | (28.5%) |
| HP_345044 | Fstl5 (FSTL5) | 45/201 | (22.3%) |
| HP_55878 | Arid5b (ARID5B) | 49/231 | (21.2%) |
| HP_484212 | Nrsn2 (NRSN2) | 60/284 | (21.1%) |
| HP_246874 | Wdr37 (WDR37) | 60/284 | (21.1%) |
| HP_302435 | Armcx1 (ARMCX1) | 61/290 | (21%) |
| HP_238455 | Gjd4 (GJD4) | 76/396 | (19.1%) |
| HP_282953 | BC050789 (FLJ23049) | 70/396 | (17.6%) |
| HP_488528 | Armcx6 (ARMCX6) | 41/284 | (14.4%) |
| HP_293818 | Fxr1h (FXR1) | 31/231 | (13.4%) |
| HP_524 | Pten (PTEN) | 34/290 | (11.7%) |
| HP_268588 | Ddx20 (DDX20) | 61/560 | (10.8%) |
| HP_420314 | Pde3b (PDE3B) | 42/396 | (10.6%) |
| HP_278543 | Glo1 (GLO1) | 29/284 | (10.2%) |
| HP_284899 | Sh2d3c (SH2D3C) | 19/201 | (9.4%) |
| HP_7310 | ITIH4 (ITIH4) | 21/231 | (9%) |
| HP_283949 | Rnf125 (RNF125) | 26/290 | (8.9%) |
| HP_309846 | 6530418L21Rik (C1orf183) | 18/201 | (8.9%) |
| HP_322570 | 1810063B07Rik (C6orf64) | 32/396 | (8%) |
| HP_353921 | Set (SET) | 32/396 | (8%) |
| HP_522 | Pten (PTEN) | 18/231 | (7.7%) |
| HP_22132 | Fgf6 (FGF6) | 43/560 | (7.6%) |
| HP_268149 | Cc2d1a (CC2D1A) | 15/201 | (7.4%) |
| HP_299169 | 1700010M22Rik (C20orf79) | 16/231 | (6.9%) |
| HP_307744 | Olfr732 (OR4N4) | 19/284 | (6.6%) |
| HP_272971 | Mapk4 (MAPK4) | 19/290 | (6.5%) |
| HP_479490 | Olfr734 (OR4M1) | 17/284 | (5.9%) |
| HP_485412 | Gm276 (FAM81B) | 22/396 | (5.5%) |
| HP_270196 | Set (SET) | 31/560 | (5.5%) |
| HP_265589 | Btbd9 (BTBD9) | 29/560 | (5.1%) |
| HP_410182 | Mcart1 (MCART1) | 20/396 | (5%) |
| HP_492873 | Fstl5 (FSTL5) | 20/396 | (5%) |

TABLE 2
Distribution of shRNA Specific Sequence Reads in DNA from 31 Tumors

| | | Tumor ID | | | |
|---|---|---|---|---|---|
| | Tumor | (shRNA | | | No of |
| Pool | incidence | sequence reads) | shRNA ID | Mouse (HUMAN) gene name | reads |
| 1 | 5/8 | 1-1 (68 reads) | HP_465354 | Pten (PTEN) | 45 |
| | | | HP_55878 | Arid5b (ARID5B) | 10 |
| | | | HP_17666 | Fxr1h (FXR1) | 6 |
| | | 1-2 (79 reads) | HP_465354 | Pten (PTEN) | 20 |
| | | | HP_55878 | Arid5b (ARID5B) | 18 |
| | | | HP_293818 | Fxr1h (FXR1) | 15 |
| | | | HP_48964 | Sh3glb2 (SH3GLB2) | 8 |
| | | | HP_299169 | 1700010M22Rik (C20orf79) | 8 |
| | | 1-3 (84 reads) | HP_55878 | Arid5b (ARID5B) | 21 |
| | | | HP_7310 | ITIH4 (ITIH4) | 16 |
| | | | HP_293818 | Fxr1h (FXR1) | 16 |
| | | | HP_522 | Pten (PTEN) | 13 |
| 5 | 6/8 | 5-1 (73 reads) | HP_524 | Pten (PTEN) | 30 |
| | | | HP_283949 | Rnf125 (RNF125) | 11 |
| | | | HP_295488 | Itpr1 (ITPR1) | 7 |
| | | 5-2 (81 reads) | HP_89078 | Armcx2 (ARMCX2) | 34 |
| | | | HP_272971 | Mapk4 (MAPK4) | 14 |
| | | | HP_283949 | Rnf125 (RNF125) | 12 |
| | | | HP_288339 | Rin2 (RIN2) | 8 |
| | | 5-3 (77 reads) | HP_89078 | Armcx2 (ARMCX2) | 49 |
| | | | HP_291484 | St6galnac4 (ST6GALNAC4) | 6 |
| | | 5-4 (62 reads) | HP_302435 | Armcx1 (ARMCX1) | 59 |
| 8 | 4/8 | 8-1 (84 reads) | HP_345037 | Wdr49 (WDR49) | 38 |
| | | | HP_253140 | Fgf6 (FGF6) | 33 |
| | | | HP_415905 | Zmym2 (ZMYM2) | 7 |
| | | 8-2 (86 reads) | HP_253140 | Fgf6 (FGF6) | 56 |
| | | | HP_345037 | Wdr49 (WDR49) | 28 |
| | | 8-3 (90 reads) | HP_253140 | Fgf6 (FGF6) | 48 |
| | | | HP_345037 | Wdr49 (WDR49) | 41 |
| | | 8-4 (82 reads) | HP_253140 | Fgf6 (FGF6) | 38 |
| | | | HP_345037 | Wdr49 (WDR49) | 30 |
| | | | HP_415905 | Zmym2 (ZMYM2) | 6 |
| | | | HP_229153 | Polr3f (POLR3F) | 6 |
| 9 | 7/8 | 9-1 (69 reads) | HP_484212 | Nrsn2 (NRSN2) | 23 |
| | | | HP_488528 | Armcx6 (ARMCX6) | 15 |
| | | | HP_246874 | Wdr37 (WDR37) | 8 |
| | | 9-2 (64 reads) | HP_484212 | Nrsn2 (NRSN2) | 36 |
| | | | HP_353467 | Ccny (CCNY) | 6 |
| | | 9-3 (77 reads) | HP_246874 | Wdr37 (WDR37) | 31 |
| | | | HP_479490 | Olfr734 (OR4M1) | 15 |
| | | | HP_307744 | Olfr732 (OR4N4) | 14 |
| | | 9-4 (74 reads) | HP_488528 | Armcx6 (ARMCX6) | 20 |
| | | | HP_246874 | Wdr37 (WDR37) | 19 |
| 10 | 8/8 | 10-1 (72 reads) | HP_76455 | Xpo4 (XPO4) | 35 |
| | | | HP_477212 | Serpini2 (SERPINI2) | 7 |
| | | | HP_268588 | Ddx20 (DDX20) | 7 |
| | | | HP_265589 | Btbd9 (BTBD9) | 6 |
| | | 10-2 (53 reads) | HP_22132 | Fgf6 (FGF6) | 43 |
| | | 10-3 (86 reads) | HP_76455 | Xpo4 (XPO4) | 68 |
| | | | HP_268588 | Ddx20 (DDX20) | 16 |
| | | 10-4 (89 reads) | HP_76455 | Xpo4 (XPO4) | 78 |
| | | | HP_268588 | Ddx20 (DDX20) | 10 |
| | | 10-5 (85 reads) | HP_76455 | Xpo4 (XPO4) | 72 |
| | | | HP_268588 | Ddx20 (DDX20) | 12 |
| | | 10-6 (89 reads) | HP_76455 | Xpo4 (XPO4) | 48 |
| | | | HP_265589 | Btbd9 (BTBD9) | 23 |
| | | | HP_268588 | Ddx20 (DDX20) | 9 |
| | | 10-7 (86 reads) | HP_76455 | Xpo4 (XPO4) | 50 |
| | | | HP_270196 | Set (SET) | 29 |
| | | | HP_268588 | Ddx20 (DDX20) | 7 |
| 11 | 6/8 | 11-1 (69 reads) | HP_284899 | Sh2d3c (SH2D3C) | 19 |
| | | | HP_278543 | Glo1 (GLO1) | 14 |
| | | | HP_268149 | Cc2d1a (CC2D1A) | 11 |
| | | | HP_345044 | Fstl5 (FSTL5) | 10 |
| | | 11-2 (62 reads) | HP_309846 | 6530418L21Rik (C1orf183) | 15 |
| | | | HP_278543 | Glo1 (GLO1) | 13 |
| | | | HP_345044 | Fstl5 (FSTL5) | 7 |
| | | | HP_278031 | Maob (MAOB) | 6 |
| | | 11-3 (70 reads) | HP_278543 | Glo1 (GLO1) | 29 |
| | | | HP_345044 | Fstl5 (FSTL5) | 28 |
| 12 | 5/8 | 12-1 (69 reads) | HP_420314 | Pde3b (PDE3B) | 13 |
| | | | HP_353921 | Set (SET) | 12 |
| | | | HP_353122 | Qtrtd1 (QTRTD1) | 11 |
| | | | HP_271204 | Bmp2k (BMP2K) | 6 |
| | | 12-1 (59 reads) | HP_322570 | 1810063B07Rik (C6orf64) | 21 |
| | | | HP_420314 | Pde3b (PDE3B) | 17 |
| | | | HP_485412 | Gm276 (FAM81B) | 11 |
| | | | HP_353921 | Set (SET) | 10 |
| | | 12-3 (77 reads) | HP_238455 | Gjd4 (GJD4) | 20 |
| | | | HP_477682 | Cryz (CRYZ) | 13 |
| | | | HP_420314 | Pde3b (PDE3B) | 11 |
| | | | HP_322570 | 1810063B07Rik (C6orf64) | 10 |
| | | | HP_485412 | Gm276 (FAM81B) | 7 |
| | | | HP_353921 | Set (SET) | 6 |
| | | 12-4 (65 reads) | HP_492873 | Fstl5 (FSTL5) | 20 |
| | | | HP_410182 | Mcart1 (MCART1) | 17 |
| | | | HP_483780 | Erich1 (ERICH1) | 10 |
| | | 12-5 (60 reads) | HP_282953 | ZBBX (ZBBX) | 31 |
| | | | HP_238455 | Gjd4 (GJD4) | 26 |
| | | 12-6 (66 reads) | HP_282953 | ZBBX (ZBBX) | 39 |
| | | | HP_238455 | Gjd4 (GJD4) | 26 |

Distribution of shRNA specific sequence reads in DNA from 31 different tumors. Listed all shRNAs with >5 reads.
Detailed shRNA information is available at Cold Spring Harbor Laboratories website, using the given shRNA ID.

TABLE 3
58 Focal Deletions in HCC-Containing 362 Genes

| | | Start | Size | | Gene | |
|---|---|---|---|---|---|---|
| Cnt | Chr | (Mb) | (Mb) | T/C | Cnt | Representative Genes |
| 7 | 12 | 123.1 | 1.03 | | 7 | NCOR2 SCARB1 UBC FAM101A AACS BRI3BP DHX37 |
| 6 | 15 | 18.4 | 1.61 | Cent. | 5 | OR4M2 OR4N4 LOC400968 POTE15 LOC283755 |
| 5 | 9 | 21.5 | 1.44 | | 4 | CDKN2A CDKN2B DMRTA1 MTAP |
| 4 | 23 | 0.9 | 1.55 | Telo. | 0 | |
| 4 | 9 | 127.6 | 1.46 | | 46 | WDR34 C9orf16 AK1 FAM73B PKN3 C9orf114 DNM1 ENDOG FAM102A SPTAN1 |
| | | | | | | ZDHHC12 CDK9 |
| 4 | 3 | 0.1 | 0.60 | Telo. | 1 | CHL1 |
| 4 | 4 | 187.8 | 0.22 | Telo. | 2 | FAT MTNR1A |
| 2 | 17 | 10.2 | 2.60 | | 15 | MYH3 MYH13 MYOCD SCO1 MYH8 FLJ45455 ZNF18 LOC388335 DNAH9 MYH2 |
| | | | | | | MAP2K4 MYH1 |
| 2 | 23 | 99.5 | 2.36 | | 35 | BTK TMSL8 ARMCX3 TCEAL2 TSPAN6 GLA TCEAL6 NXF2 TNMD TMEM35 ZMAT1 |
| | | | | | | ARMCX1 |
| 2 | 13 | 80.7 | 2.24 | | 0 | |
| 2 | 2 | 237.5 | 1.92 | | 17 | LOC339768 |
| 2 | 23 | 43.0 | 1.87 | | 8 | MAOA NDP DUSP21 CXorf36 EFHC2 FUNDC1 MAOB UTX |
| 2 | 3 | 167.0 | 1.72 | | 4 | FLJ23049 SERPINI2 BCHE WDR49 |
| 2 | 10 | 89.3 | 1.62 | | 11 | STAMBPL1 LIPF C10orf59 MINPP1 FAS ACTA2 ATAD1 ANKRD22 LIPL1 PAPSS2 |
| 2 | 8 | 0.2 | 1.17 | Telo. | 4 | ERICH1 C8orf42 LOC389607 FBXO25 |
| 2 | 18 | 64.2 | 1.14 | | 3 | C18orf14 DOK6 TXNDC10 |
| 2 | 14 | 105.3 | 0.93 | | 1 | KIAA0125 |
| 2 | 1 | 111.7 | 0.87 | | 6 | C1orf183 DDX20 C1orf162 RAP1A KCND3 ADORA3 |
| 2 | 6 | 113.3 | 0.80 | | 0 | |
| 2 | 5 | 0.1 | 0.79 | Telo. | 12 | TPPP SDHA ZDHHC11 EXOC3 LOC133957 SLC9A3 AHRR PDCD6 KIAA1909 BRD9 |
| | | | | | | Cep72 |
| 2 | 4 | 21.2 | 0.53 | | 1 | KCNIP4 |
| 2 | 5 | 58.8 | 0.51 | | 1 | PDE4D |
| 2 | 23 | 7.8 | 0.39 | | 1 | VCX2 |
| 1 | 10 | 0.1 | 2.30 | Telo. | 11 | GTPBP4 IDI1 C10orf108 WDR37 ADARB2 LARP5 LOC399706 ZMYND11 DIP2C |
| | | | | | | C10orf110 |
| 1 | 5 | 1.8 | 2.24 | | 6 | CEI NDUFS6 IRX2 IRX1 IRX4 MRPL36 |
| 1 | 3 | 162.6 | 2.23 | | 0 | |
| 1 | 3 | 180.5 | 2.13 | | 11 | FXR1 PEX5L DNAJC19 MRPL47 GNB4 MFN1 NDUFB5 TTC14 ACTL6A ZNF639 |
| 1 | 1 | 29.2 | 2.10 | | 9 | SDC3 TTMB PUM1 SFRS4 MATN1 PTPRU MECR EPB41 LAPTM5 |
| 1 | 13 | 18.3 | 2.04 | | 14 | HSMPP8 ZNF198 IFT88 IL17D ZMYM5 TPTE2 LOC221143 PSPC1 GJB6 GJA3 XPO4 |
| | | | | | | CRYL1 |
| 1 | 10 | 35.6 | 2.02 | | 4 | CX40.1 C10orf9 FZD8 ANKRD30A |
| 1 | 4 | 79.8 | 1.70 | | 7 | BMP2K ANXA3 ANTXR2 PAQR3 FGF5 PRDM8 GK2 |
| 1 | 18 | 46.2 | 1.62 | | 6 | MRO MAPK4 ME2 ELAC1 SMAD4 RKHD2 |
| 1 | 6 | 37.9 | 1.60 | | 10 | KCNK5 GLP1R KIF6 TEX27 DNAH8 KCNK17 GLO1 BTBD9 KCNK16 C6orf64 |
| 1 | 20 | 18.4 | 1.57 | | 9 | HSPC072 SLC24A3 C20orf79 C20orf12 HARS2 RBBP9 RIN2 SEC23B POLR3F |
| 1 | 4 | 161.6 | 1.56 | | 1 | FSTL5 |
| 1 | 21 | 22.2 | 1.42 | | 0 | |
| 1 | 18 | 26.7 | 1.37 | | 14 | DSC3 KIAA1012 RNF138 TTR RNF125 DSG4 DSG3 DSC1 MEP1B DSG2 MCART2 DSC2 |
| 1 | 3 | 195.0 | 1.16 | | 6 | LSG1 FAM43A TMEM44 HES1 LRRC15 GP5 |
| 1 | 5 | 25.3 | 1.16 | | 0 | |
| 1 | 3 | 2.2 | 1.02 | | 4 | CRBN CNTN4 IL5RA TRNT1 |
| 1 | 3 | 115.2 | 0.98 | | 6 | DRD3 QTRTD1 FLJ39873 ZNF80 KIAA1407 ZBTB20 |
| 1 | 5 | 145.6 | 0.91 | | 4 | TCERG1 POU4F3 GPR151 PPP2R2B |
| 1 | 5 | 119.3 | 0.90 | | 1 | LOC51334 |
| 1 | 3 | 97.6 | 0.88 | | 0 | |
| 1 | 13 | 33.8 | 0.87 | | 1 | NBEA |
| 1 | 1 | 20.1 | 0.81 | | 14 | PLA2G2F |
| 1 | 3 | 4.6 | 0.78 | | 4 | ITPR1 EDEM1 BHLHB2 ARL8B |
| 1 | 23 | 33.7 | 0.72 | | 1 | FAM47A |
| 1 | 10 | 11.1 | 0.69 | | 2 | C10orf31 CUGBP2 |
| 1 | 12 | 4.2 | 0.52 | | 10 | C12orf5 FGF6 AKAP3 NDUFA9 C12orf4 GALNT8 CCND2 FGF23 DYRK4 RAD51AP1 |
| 1 | 6 | 157.0 | 0.50 | | 1 | ARID1B |
| 1 | 11 | 14.3 | 0.48 | | 4 | RRAS2 COPB PSMA1 PDE3B |
| 1 | 12 | 77.6 | 0.44 | | 0 | |
| 1 | 20 | 0.0 | 0.34 | Telo. | 12 | C20orf18 |
| 1 | 1 | 230.9 | 0.33 | | 2 | IRF2BP2 TARBP1 |
| 1 | 21 | 39.2 | 0.24 | | 1 | FLJ45139 |
| 1 | 10 | 63.2 | 0.22 | | 2 | C10orf107 ARID5B |
| 1 | 13 | 50.7 | 0.10 | | 1 | FLJ30707 |

TABLE 4
shRNAs Found in >5% of Total Reads in Each Given Pool

| | | shRNA specific | |
|---|---|---|---|
| | Mouse (HUMAN) gene | reads/total reads | |
| Hairpin ID | name | of pool | |
| HP_76455* | Xpo4 (XPO4) | 351/560 | (62.6%) |
| HP_253140* | Fgf6 (FGF6) | 175/342 | (51.1%) |
| HP_345037* | Wdr49 (WDR49) | 137/342 | (40%) |
| HP_89078* | Armcx2 (ARMCX2) | 88/290 | (30.3%) |
| HP_465354* | Pten (PTEN) | 66/231 | (28.5%) |
| HP_345044* | Fstl5 (FSTL5) | 45/201 | (22.3%) |
| HP_55878 | Arid5b (ARID5B) | 49/231 | (21.2%) |
| HP_484212* | Nrsn2 (NRSN2) | 60/284 | (21.1%) |
| HP_246874* | Wdr37 (WDR37) | 60/284 | (21.1%) |
| HP_302435* | Armcx1 (ARMCX1) | 61/290 | (21%) |
| HP_238455* | Gjd4 (GJD4) | 76/396 | (19.1%) |
| HP_282953* | Zbbx (ZBBX) | 70/396 | (17.6%) |
| HP_278543* | Glo1 (GLO1) | 29/201 | (14.4%) |
| HP_488528 | Armcx6 (ARMCX6) | 41/284 | (14.4%) |
| HP_293818 | Fxr1h (FXR1) | 31/231 | (13.4%) |
| HP_524* | Pten (PTEN) | 34/290 | (11.7%) |
| HP_268588* | Ddx20 (DDX20) | 61/560 | (10.8%) |
| HP_420314 | Pde3b (PDE3B) | 42/396 | (10.6%) |
| HP_284899 | Sh2d3c (SH2D3C) | 19/201 | (9.4%) |
| HP_7310 | ITIH4 (ITIH4) | 21/231 | (9%) |
| HP_283949 | Rnf125 (RNF125) | 26/290 | (8.9%) |
| HP_309846 | 6530418L21Rik (C1orf183) | 18/201 | (8.9%) |
| HP_322570 | 1810063B07Rik (C6orf64) | 32/396 | (8%) |
| HP_353921* | Set (SET) | 32/396 | (8%) |
| HP_522 | Pten (PTEN) | 18/231 | (7.7%) |
| HP_22132* | Fgf6 (FGF6) | 43/560 | (7.6%) |
| HP_268149 | Cc2d1a (CC2D1A) | 15/201 | (7.4%) |
| HP_299169 | 1700010M22Rik (C20orf79) | 16/231 | (6.9%) |
| HP_307744 | Olfr732 (OR4N4) | 19/284 | (6.6%) |
| HP_272971 | Mapk4 (MAPK4) | 19/290 | (6.5%) |
| HP_479490 | Olfr734 (OR4M1) | 17/284 | (5.9%) |
| HP_485412 | Gm276 (FAM81B) | 22/396 | (5.5%) |
| HP_270196* | Set (SET) | 31/560 | (5.5%) |
| HP_265589* | Btbd9 (BTBD9) | 29/560 | (5.1%) |
| HP_410182 | Mcart1 (MCART1) | 20/396 | (5%) |
| HP_492873 | Fstl5 (FSTL5) | 20/396 | (5%) |

shRNAs found in >5% of the total reads in each given pool.
*denotes shRNAs selected for validation.

TABLE 5
Counts of 12 Scored HCC Deletions in Two Breast Cancer ROMA Data Sets

| | Scandavian | MSKCC | Gene | |
|---|---|---|---|---|
| Chr | Set (247) | Set (125) | Count | Genes |
| 1 | yes 1 | edge | 6 | C1orf183, DDX20, C1orf162, RAP1A, KCND3, |
| | | | | ADORA3 |
| 3 | no | no | 4 | ZBBX, SERPINI2, BCHE, WDR49 |
| 4 | yes 1 | no | 1 | FSTL5 |
| 12 | yes 1 | yes 7 | 10 | C12orf5, FGF6, AKAP3, NDUFA9, C12orf4, GALNT8, |
| | | | | CCND2, FGF23, DYRK4, RAD51AP1 |
| 23 | nd | nd | 35 | BTK, TMSL8, ARMCX3, TCEAL2, TSPAN6, GLA, |
| | | | | TCEAL6, NXF2, TNMD, TMEM35, XKRX, ZMAT1, |
| | | | | ARMCX1, NXF5, GPRASP2, NOX1, DRP2, ARMCX4, |
| | | | | BHLHB9, NGFRAP1L1, FSHPRH1, GPRASP1, |
| | | | | HNRPH2, SYTL4, dJ341D10.1, TIMM8A, SRPX2, |
| | | | | CXorf34, RPL36A, ARMCX5, TAF7L, CSTF2, |
| | | | | ARL13A, ARMCX6, ARMCX2 |
| 9 | yes 2 | yes 2 | 46 | WDR34, C9orf16, AK1, FAM73B, PKN3, C9orf114, |
| | | | | DNM1, ENDOG, FAM102A, SPTAN1, DPM2, |
| | | | | ZDHHC12, CDK9, FPGS, CCBL1, PIP5KL1, LCN2, |
| | | | | TRUB2, CEECAM1, GOLGA2, COQ4, LOC389791, |
| | | | | SH3GLB2, C9orf90, TBC1D13, NUP188, ZYG11BL, |
| | | | | LRRC8A, TGES2, C9orf74, IER5L, PHYHD1, GLE1L, |
| | | | | CRAT, ST6GALNAC4, DOLPP1, SLC25A25, ENG, |
| | | | | SLC27A4, SET, CIZ1, SH2D3C, PPP2R4, MEM15, |
| | | | | ODF2, ST6GALNAC6, |
| 13 | yes 8 | yes 2 | 14 | HSMPP8, ZNF198, IFT88, IL17D, ZMYM5, TPTE2, |
| | | | | LOC221143, PSPC1, GJB6, GJA3, GJB2, XPO4, |
| | | | | CRYL1, TUBA2 |
| 20 | yes 4 | yes 8 | 12 | DEFB127, SOX12, TRIB3, C20orf96, TBC1D20, |
| | | | | DEFB129, NRSN2, DEFB32, DEFB128, CCHC3, |
| | | | | DEFB126, C20orf18 |
| 10 | no | yes 1 | 4 | GJD4, C10orf9, FZD8, ANKRD30A |
| 6 | yes 3 | yes 5 | 10 | KCNK5, GLP1R, KIF6, TEX27, DNAH8, KCNK17, |
| | | | | GLO1, BTBD9, KCNK16, C6orf64 |
| 10 | yes 4 | yes 4 | 11 | STAMBPL1, LIPF, C10orf59, MINPP1, FAS, ACTA2, |
| | | | | ATAD1, ANKRD22, LIPL1, PAPSS2, PTEN |
| 10 | yes 1 | yes 5 | 11 | GTPBP4, IDI1, C10orf108, WDR37, ADARB2, |
| | | | | LARP5, LOC399706, ZMYND11, DIP2C, C10orf110, |
| | | | | IDI2 |

Counts of 12 scored HCC deletions detected in two breast cancer ROMA data sets. Validated genes are indicated in bold type.

TABLE 6
Clinical Study in Breast Cancer Patients

| | | Number with |
|---|---|---|
| | Total Number | XPO4 deletion |
| Survivor | 172 | 36 |
| Non-survivor | 78 | 20 |

* Chi-square = 4.33
** p = 0.038

TABLE 7
Tumor Suppressor Genes Identified in Study

| | Mouse (HUMAN) gene name |
|---|---|
| 1 | Xpo4 (XPO4) |
| 2 | Fgf6 (FGF6) |
| 3 | Wdr49 (WDR49) |
| 4 | Armcx2 (ARMCX2) |
| 5 | Fstl5 (FSTL5) |
| 6 | Nrsn2 (NRSN2) |
| 7 | Wdr37 (WDR37) |
| 8 | Armcx1 (ARMCX1) |
| 9 | Gjd4 (GJD4) |
| 10 | Zbbx (ZBBX) |
| 11 | Glo1 (GLO1) |
| 12 | Ddx20 (DDX20) |
| 13 | Set (SET) |
| 14 | Btbd9 (BTBD9) |

What is claimed is:

1. A method of identifying a tumor suppressor gene, the method comprising:
(a) identifying a genomic deletion in a population of cancer cells;
(b) cloning one or more shRNAs that target sequences located within the genomic deletion into an expression vector;
(c) pooling the expression vectors generated in step (b) to generate an shRNA library;
(d) transforming the shRNA library into mouse embryonic hepatocytes;
(e) introducing the transformed mouse embryonic hepatocytes into mice;
(f) amplifying an shRNA from a tumor cell formed in the mice; and
(g) identifying a genomic sequence to which the shRNA is complementary;
thereby identifying a tumor suppressor gene.

2. The method of claim 1, wherein the genomic deletion is a focal genomic deletion.

3. The method of claim 1, wherein the genomic deletion is identified using representational oligonucleotide microarray analysis.

4. The method of claim 1, wherein the expression vector is a viral vector.

5. The method of claim 1, wherein the expression vector further comprises a reporter.

6. The method of claim 5, wherein the reporter is green fluorescent protein (GFP).

7. The method of claim 1, wherein the mouse embryonic hepatocytes are p53−/−;Myc.

8. The method of claim 1, wherein the step of transforming the mouse embryonic hepatocytes is performed by infection.

9. The method of claim 1, wherein step of transforming the mouse embryonic hepatocytes comprises is performed by transfection.

10. A method for generating an shRNA library useful for identifying tumor suppressor genes, comprising:
(a) identifying one or more genomic deletions in a population of cancer cells;
(b) cloning one or more shRNAs that target sequences located within the one or more genomic deletions into an expression vector;
(c) pooling the expression vectors generated in step (b) to generate an shRNA library;
thereby generating an shRNA library useful for identifying tumor suppressor genes.

11. The method of claim 10, wherein the genomic deletion is a focal genomic deletion.

12. An shRNA library consisting essentially of shRNA molecules that target mRNAs transcribed from one or more genes located within one or more cancer-associated genomic deletions.

13. The shRNA library of claim 12, wherein the genes are selected from the 362 genes listed in Table 3.

14. The shRNA library of claim 12, wherein the cancer-associated genomic deletions are selected from the 58 focal deletions listed in Table 3.

15. A method of inhibiting cell proliferation, the method comprising upregulating expression of a gene selected from the group consisting of XPO4, FGF6, WDR49, ARMCX2, FSTL5, NRSN2, WDR37, ARMCX1, GJD4, ZBBX, GLO1, DDX20, SET, and BTBD9 in the cell, thereby inhibiting cell proliferation.

16. The method of claim 15, wherein the gene is XPO4.

17. The method of claim 16, wherein upregulating XPO4 comprises introducing into the cell a nucleic acid molecule encoding a physiologically active XPO4 polypeptide.

18. The method of claim 15, wherein the cell is a cancer cell.

19. The method of claim 15, wherein the cell is a liver cancer cell.

20. The method of claim 15, wherein the cell is a breast cancer cell.

21. A method of inhibiting cell proliferation, the method comprising administering to a cell a a physiologically active polypeptide encoded by a gene selected from the group consisting of XPO4, FGF6, WDR49, ARMCX2, FSTL5, NRSN2, WDR37, ARMCX1, GJD4, ZBBX, GLO1, DDX20, SET, and BTBD9, or an analog thereof, thereby inhibiting cell proliferation.

22. The method of claim 21, comprising administering to the cell a physiologically active XPO4 polypeptide, or an analog thereof

23. The method of claim 21, wherein the cell is a cancer cell.

24. The method of claim 21, wherein the cell is a liver cancer cell.

25. The method of claim 21, wherein the cell is a breast cancer cell.

26. A method for determining whether a subject is suffering from, or at risk of developing, liver or breast cancer, the method comprising obtaining genomic DNA from the subject and determining whether the genomic DNA contains a deletion of one or more of genes selected from the group consisting of XPO4, FGF6, WDR49, ARMCX2, FSTL5, NRSN2, WDR37, ARMCX1, GJD4, ZBBX, GLO1, DDX20, SET, and BTBD9, whereby a deletion of one or more of the genes indicates that the subject is suffering from, or at risk of developing, liver or breast cancer.

27. The method of claim 26, wherein the gene is the XPO4 gene.

28. A method for determining whether a subject is suffering from, or at risk of developing, liver or breast cancer, the method comprising obtaining a sample comprising mRNA from the subject, and measuring the level of an mRNA selected from the group consisting of XPO4, FGF6, WDR49, ARMCX2, FSTL5, NRSN2, WDR37, ARMCX1, GJD4, ZBBX, GLO1, DDX20, SET, and BTBD9 mRNAs in the sample, whereby a level of expression of one or more of the mRNAs that is lower than the level of expression of the mRNA in a control subject, indicates that the subject is suffering from, or at risk of developing, liver or breast cancer.

29. The method of claim 28, wherein the mRNA is XPO4 mRNA.

30. A method for determining whether a subject is suffering from, or at risk of developing, liver or breast cancer, the method comprising obtaining a sample from the subject and measuring the level of a protein selected from the group consisting of the proteins encoded by the XPO4, FGF6, WDR49, ARMCX2, FSTL5, NRSN2, WDR37, ARMCX1, GJD4, ZBBX, GLO1, DDX20, SET, and BTBD9 genes in the sample, whereby a level of the protein that is lower than the level of the same protein in a control subject, indicates that the subject is suffering from, or at risk of developing, liver or breast cancer.

31. The method of claim 30, wherein the protein is encoded by the XPO4 gene.

32. A method of inhibiting cell proliferation, the method comprising inhibiting the expression of EIF5A2 in the cell.

33. The method of claim 32, comprising introducing into the cell an interfering RNA molecule that targets EIF5A2.

34. The method of claim 33, wherein the interfering RNA molecule is an shRNA or an siRNA.

35. A method for determining whether a subject is suffering from, or at risk of developing, liver or breast cancer, the method comprising obtaining genomic DNA from the subject and determining whether the genomic DNA contains an amplification of the EIF5A2 gene, whereby amplification of the EIF5A2 gene indicates that the subject is suffering from, or at risk of developing, liver or breast cancer.

36. A method for determining whether a subject is suffering from, or at risk of developing, liver or breast cancer, the method comprising obtaining a sample comprising mRNA from the subject and measuring the level of EIF5A2 mRNA in the sample, whereby a level of EIF5A2 mRNA that is higher than the level of EIF5A2 mRNA in a control subject, indicates that the subject is suffering from, or at risk of developing, liver or breast cancer.

37. A method for determining whether a subject is suffering from, or at risk of developing, liver or breast cancer, the method comprising obtaining a sample from the subject and measuring the level of EIF5A2 protein in the sample, whereby a level of the EIF5A2 protein that is higher than the level of EIF5A2 protein in a control subject, indicates that the subject is suffering from, or at risk of developing, liver or breast cancer.

38. A method of identifying an oncogene, the method comprising:
(a) identifying a genomic amplification in a population of cancer cells;
(b) cloning one or more cDNAs encoded by genes located within the genomic amplification into an expression vector;
(c) pooling the expression vectors generated in step (b) to generate a library of cDNAs;
(d) transforming the library of cDNAs into mouse embryonic hepatocytes;
(e) introducing the transformed mouse embryonic hepatocytes into mice;
(f) amplifying a cDNA present in the library of cDNAs from tumor cell formed in the mice; and
(g) determining the sequence of the cDNA;
thereby identifying an oncogene.

39. A method of identifying a tumor suppressor gene, the method comprising:
(a) identifying a genomic region that is methylated in a population of cancer cells;
(b) cloning one or more shRNAs that target sequences located within the methylated region into an expression vector;
(c) pooling the expression vectors generated in step (b) to generate an shRNA library;
(d) transforming the shRNA library into mouse embryonic hepatocytes;
(e) introducing the transformed mouse embryonic hepatocytes into mice;
(f) amplifying a shRNA from a tumor cell formed in the mice; and
(g) identifying a genomic sequence to which the shRNA is complementary;
thereby identifying a tumor suppressor gene.

40. A method for generating a cDNA library useful for identifying oncogenes, comprising:
(a) identifying one or more genomic amplifications in a population of cancer cells;
(b) cloning one or more cDNAs encoded by genes located within the one or more genomic amplifications into an expression vector;
(c) pooling the expression vectors generated in step (b) to generate a cDNA library;
thereby generating a cDNA library useful for identifying oncogenes.

41. A method for generating an shRNA library useful for identifying tumor suppressor genes, comprising:
(a) identifying one or more genomic regions that are methylated in a population of cancer cells;
(b) cloning one or more shRNAs that target sequences located within the methylated region into an expression vector;
(c) pooling the expression vectors generated in step (b) to generate an shRNA library;
thereby generating an shRNA library useful for identifying tumor suppressor genes.

42. A cDNA library consisting essentially of cDNA molecules that are encoded by genes located within one or more cancer-associated genomic amplifications.

43. An shRNA library consisting essentially of shRNA molecules that target mRNAs transcribed from one or more genes that are methylated in cancer cells.

44. A composition comprising a protein encoded by gene selected from the group consisting of XPO4, FGF6, WDR49, ARMCX2, FSTL5, NRSN2, WDR37, ARMCX1, GJD4, ZBBX, GLO1, DDX20, SET, and BTBD9, or a fragment, variant, or analog thereof, wherein the protein is fused to a tumor targeting moiety.

45. The composition of claim 44, wherein the tumor targeting moiety is an antibody that binds to cancer cells.

46. The composition of claim 44, wherein the tumor targeting moiety is an antibody that binds to liver cancer cells.

47. The composition of claim 44, wherein the tumor targeting moiety is an antibody that binds to breast cancer cells.


SEQUENCE LISTING (26 sequences)

SEQ ID NO: 1 - DNA, 25 bp, Artificial Sequence
  Description of Artificial Sequence Synthetic primer
cggatcctctcaaggcaccaccttc

SEQ ID NO: 2 - DNA, 29 bp, Artificial Sequence
  Description of Artificial Sequence Synthetic Primer
gtcacgcgtttattttacacaaaggagac

SEQ ID NO: 3 - DNA, 20 bp, Artificial Sequence
  Description of Artificial Sequence Synthetic primer
agggagttgggtactgcttg

SEQ ID NO: 4 - DNA, 20 bp, Artificial Sequence
  Description of Artificial Sequence Synthetic primer
agggcacaagaccatttacc

SEQ ID NO: 5 - DNA, 19 bp, Artificial Sequence
  Description of Artificial Sequence Synthetic primer
aggggcttccagcacttat

SEQ ID NO: 6 - DNA, 20 bp, Artificial Sequence
  Description of Artificial Sequence Synthetic primer
cccagtcttggaagttgaca

SEQ ID NO: 7 - DNA, 20 bp, Artificial Sequence
  Description of Artificial Sequence Synthetic primer
atggccattgaatacagcaa

SEQ ID NO: 8 - DNA, 20 bp, Artificial Sequence
  Description of Artificial Sequence Synthetic primer
accaacggcttcttcctaga

SEQ ID NO: 9 - DNA, 20 bp, Artificial Sequence
  Description of Artificial Sequence Synthetic primer
ggaaggctcctcagacactc

SEQ ID NO: 10 - DNA, 20 bp, Artificial Sequence
  Description of Artificial Sequence Synthetic primer
ctccacttgagtcggggtat

SEQ ID NO: 11 - DNA, 20 bp, Artificial Sequence
  Description of Artificial Sequence Synthetic primer
gacaggctaacagtggctca

SEQ ID NO: 12 - DNA, 20 bp, Artificial Sequence
  Description of Artificial Sequence Synthetic primer
tctgggtagccaaggagagt

SEQ ID NO: 13 - DNA, 20 bp, Artificial Sequence
  Description of Artificial Sequence Synthetic primer
ccagtagccgactacaacca

SEQ ID NO: 14 - DNA, 20 bp, Artificial Sequence
  Description of Artificial Sequence Synthetic primer
cgatcaagaagaggcagaca

SEQ ID NO: 15 - DNA, 20 bp, Artificial Sequence
  Description of Artificial Sequence Synthetic primer
agaattgatcgccaaaatcc

SEQ ID NO: 16 - DNA, 20 bp, Artificial Sequence
  Description of Artificial Sequence Synthetic primer
ataatgcagagcctcctcgt

SEQ ID NO: 17 - DNA, 20 bp, Artificial Sequence
  Description of Artificial Sequence Synthetic primer
ccaccgatccacacagagta

SEQ ID NO: 18 - DNA, 20 bp, Artificial Sequence
  Description of Artificial Sequence Synthetic primer
ggctcctagcaccatgaaga

SEQ ID NO: 19 - DNA, 20 bp, Artificial Sequence
  Description of Artificial Sequence Synthetic primer
gggagactcttctggacagc

SEQ ID NO: 20 - DNA, 20 bp, Artificial Sequence
  Description of Artificial Sequence Synthetic primer
gagaagctaactgggcaagg

SEQ ID NO: 21 - DNA, 20 bp, Artificial Sequence
  Description of Artificial Sequence Synthetic primer
tgaagatatctgcccgtcaa

SEQ ID NO: 22 - DNA, 20 bp, Artificial Sequence
  Description of Artificial Sequence Synthetic primer
gctgtcctggagcagtgata

SEQ ID NO: 23 - DNA, 20 bp, Artificial Sequence
  Description of Artificial Sequence Synthetic primer
ggcttccagcacttacccta

SEQ ID NO: 24 - DNA, 20 bp, Artificial Sequence
  Description of Artificial Sequence Synthetic primer
accatgctttccagttttgg

SEQ ID NO: 25 - DNA, 19 bp, Artificial Sequence
  Description of Artificial Sequence Synthetic primer
ttcaacaccccagccatgt

SEQ ID NO: 26 - DNA, 18 bp, Artificial Sequence
  Description of Artificial Sequence Synthetic primer
gccagtggtacggccaga